SEQUENCE LISTING (236 sequences)

SEQ ID NO: 1 - protein, 40 aa, Artificial Sequence
  Description of Artificial Sequence Synthetic polypeptide
  MISC_FEATURE (1)..(40): This sequence may encompass 6-20 “Gly Ser” repeating units
GSGSGSGSGSGSGSGSGSGSGSGSGSGSGSGSGSGSGSGS

SEQ ID NO: 2 - protein, 39 aa, Artificial Sequence
  Description of Artificial Sequence Synthetic polypeptide
  MISC_FEATURE (1)..(39): This sequence may encompass 4-13 “Gly Gly Ser” repeating units
GGSGGSGGSGGSGGSGGSGGSGGSGGSGGSGGSGGSGGS

SEQ ID NO: 3 - protein, 40 aa, Artificial Sequence
  Description of Artificial Sequence Synthetic polypeptide
  MISC_FEATURE (1)..(40): This sequence may encompass 3-10 “Gly Gly Gly Ser” repeating units
GGGSGGGSGGGSGGGSGGGSGGGSGGGSGGGSGGGSGGGS

SEQ ID NO: 4 - protein, 40 aa, Artificial Sequence
  Description of Artificial Sequence Synthetic polypeptide
  MISC_FEATURE (1)..(40): This sequence may encompass 2-8 “Gly Gly Gly Gly Ser” repeating units
GGGGSGGGGSGGGGSGGGGSGGGGSGGGGSGGGGSGGGGS

SEQ ID NO: 5 - protein, 40 aa, Artificial Sequence
  Description of Artificial Sequence Synthetic polypeptide
  MISC_FEATURE (1)..(40): This sequence may encompass 12-40 residues
GGGGGGGGGGGGGGGGGGGGGGGGGGGGGGGGGGGGGGGG

SEQ ID NO: 6 - protein, 33 aa, Artificial Sequence
  Description of Artificial Sequence Synthetic polypeptide
  MISC_FEATURE (1)..(33): This sequence may encompass 1-3 “Gly Gly Gly Ser Gly Ser Gly Gly Gly Gly Ser” repeating units
GGGSGSGGGGSGGGSGSGGGGSGGGSGSGGGGS

SEQ ID NO: 7 - protein, 33 aa, Artificial Sequence
  Description of Artificial Sequence Synthetic polypeptide
  MISC_FEATURE (1)..(33): This sequence may encompass 1-3 “Gly Gly Gly Gly Gly Pro Gly Gly Gly Gly Pro” repeating units
GGGGGPGGGGPGGGGGPGGGGPGGGGGPGGGGP

SEQ ID NO: 8 - protein, 36 aa, Artificial Sequence
  Description of Artificial Sequence Synthetic polypeptide
  MISC_FEATURE (1)..(36): This sequence may encompass 2-6 “Gly Gly Ser Gly Gly Asp” repeating units
GGSGGDGGSGGDGGSGGDGGSGGDGGSGGDGGSGGD

SEQ ID NO: 9 - protein, 36 aa, Artificial Sequence
  Description of Artificial Sequence Synthetic polypeptide
  MISC_FEATURE (1)..(36): This sequence may encompass 2-6 “Gly Gly Ser Gly Gly Glu” repeating units
GGSGGEGGSGGEGGSGGEGGSGGEGGSGGEGGSGGE

SEQ ID NO: 10 - protein, 8 aa, Artificial Sequence
  Description of Artificial Sequence Synthetic peptide
PRFKIIGG

SEQ ID NO: 11 - protein, 8 aa, Artificial Sequence
  Description of Artificial Sequence Synthetic peptide
PRFRIIGG

SEQ ID NO: 12 - protein, 9 aa, Artificial Sequence
  Description of Artificial Sequence Synthetic peptide
SSRHRRALD

SEQ ID NO: 13 - protein, 14 aa, Artificial Sequence
  Description of Artificial Sequence Synthetic peptide
RKSSIIIRMRDVVL

SEQ ID NO: 14 - protein, 15 aa, Artificial Sequence
  Description of Artificial Sequence Synthetic peptide
SSSFDKGKYKKGDDA

SEQ ID NO: 15 - protein, 15 aa, Artificial Sequence
  Description of Artificial Sequence Synthetic peptide
SSSFDKGKYKRGDDA

SEQ ID NO: 16 - protein, 4 aa, Artificial Sequence
  Description of Artificial Sequence Synthetic peptide
IEGR

SEQ ID NO: 17 - protein, 4 aa, Artificial Sequence
  Description of Artificial Sequence Synthetic peptide
IDGR

SEQ ID NO: 18 - protein, 7 aa, Artificial Sequence
  Description of Artificial Sequence Synthetic peptide
GGSIDGR

SEQ ID NO: 19 - protein, 6 aa, Artificial Sequence
  Description of Artificial Sequence Synthetic peptide
PLGLWA

SEQ ID NO: 20 - protein, 8 aa, Artificial Sequence
  Description of Artificial Sequence Synthetic peptide
GPQGIAGQ

SEQ ID NO: 21 - protein, 8 aa, Artificial Sequence
  Description of Artificial Sequence Synthetic peptide
GPQGLLGA

SEQ ID NO: 22 - protein, 5 aa, Artificial Sequence
  Description of Artificial Sequence Synthetic peptide
GIAGQ

SEQ ID NO: 23 - protein, 8 aa, Artificial Sequence
  Description of Artificial Sequence Synthetic peptide
GPLGIAGI

SEQ ID NO: 24 - protein, 40 aa, Artificial Sequence
  Description of Artificial Sequence Synthetic polypeptide
  MOD_RES (2)..(2): Ser, Asp, Glu, Thr or Pro
  MOD_RES (4)..(4): Ser, Asp, Glu, Thr or Pro
  MOD_RES (6)..(6): Ser, Asp, Glu, Thr or Pro
  MOD_RES (8)..(8): Ser, Asp, Glu, Thr or Pro
  MOD_RES (10)..(10): Ser, Asp, Glu, Thr or Pro
  MOD_RES (12)..(12): Ser, Asp, Glu, Thr or Pro
  MOD_RES (14)..(14): Ser, Asp, Glu, Thr or Pro
  MOD_RES (16)..(16): Ser, Asp, Glu, Thr or Pro
  MOD_RES (18)..(18): Ser, Asp, Glu, Thr or Pro
  MOD_RES (20)..(20): Ser, Asp, Glu, Thr or Pro
  MOD_RES (22)..(22): Ser, Asp, Glu, Thr or Pro
  MOD_RES (24)..(24): Ser, Asp, Glu, Thr or Pro
  MOD_RES (26)..(26): Ser, Asp, Glu, Thr or Pro
  MOD_RES (28)..(28): Ser, Asp, Glu, Thr or Pro
  MOD_RES (30)..(30): Ser, Asp, Glu, Thr or Pro
  MOD_RES (32)..(32): Ser, Asp, Glu, Thr or Pro
  MOD_RES (34)..(34): Ser, Asp, Glu, Thr or Pro
  MOD_RES (36)..(36): Ser, Asp, Glu, Thr or Pro
  MOD_RES (38)..(38): Ser, Asp, Glu, Thr or Pro
  MOD_RES (40)..(40): Ser, Asp, Glu, Thr or Pro
  See specification as filed for detailed description of substitutions and preferred embodiments
GXGXGXGXGXGXGXGXGXGXGXGXGXGXGXGXGXGXGXGX

SEQ ID NO: 25 - protein, 39 aa, Artificial Sequence
  Description of Artificial Sequence Synthetic polypeptide
  MOD_RES (3)..(3): Ser, Asp, Glu, Thr or Pro
  MOD_RES (6)..(6): Ser, Asp, Glu, Thr or Pro
  MOD_RES (9)..(9): Ser, Asp, Glu, Thr or Pro
  MOD_RES (12)..(12): Ser, Asp, Glu, Thr or Pro
  MOD_RES (15)..(15): Ser, Asp, Glu, Thr or Pro
  MOD_RES (18)..(18): Ser, Asp, Glu, Thr or Pro
  MOD_RES (21)..(21): Ser, Asp, Glu, Thr or Pro
  MOD_RES (24)..(24): Ser, Asp, Glu, Thr or Pro
  MOD_RES (27)..(27): Ser, Asp, Glu, Thr or Pro
  MOD_RES (30)..(30): Ser, Asp, Glu, Thr or Pro
  MOD_RES (33)..(33): Ser, Asp, Glu, Thr or Pro
  MOD_RES (36)..(36): Ser, Asp, Glu, Thr or Pro
  MOD_RES (39)..(39): Ser, Asp, Glu, Thr or Pro
  See specification as filed for detailed description of substitutions and preferred embodiments
GGXGGXGGXGGXGGXGGXGGXGGXGGXGGXGGXGGXGGX

SEQ ID NO: 26 - protein, 40 aa, Artificial Sequence
  Description of Artificial Sequence Synthetic polypeptide
  MOD_RES (4)..(4): Ser, Asp, Glu, Thr or Pro
  MOD_RES (8)..(8): Ser, Asp, Glu, Thr or Pro
  MOD_RES (12)..(12): Ser, Asp, Glu, Thr or Pro
  MOD_RES (16)..(16): Ser, Asp, Glu, Thr or Pro
  MOD_RES (20)..(20): Ser, Asp, Glu, Thr or Pro
  MOD_RES (24)..(24): Ser, Asp, Glu, Thr or Pro
  MOD_RES (28)..(28): Ser, Asp, Glu, Thr or Pro
  MOD_RES (32)..(32): Ser, Asp, Glu, Thr or Pro
  MOD_RES (36)..(36): Ser, Asp, Glu, Thr or Pro
  MOD_RES (40)..(40): Ser, Asp, Glu, Thr or Pro
  See specification as filed for detailed description of substitutions and preferred embodiments
GGGXGGGXGGGXGGGXGGGXGGGXGGGXGGGXGGGXGGGX

SEQ ID NO: 27 - protein, 40 aa, Artificial Sequence
  Description of Artificial Sequence Synthetic polypeptide
  MOD_RES (5)..(5): Ser, Asp, Glu, Thr or Pro
  MOD_RES (10)..(10): Ser, Asp, Glu, Thr or Pro
  MOD_RES (15)..(15): Ser, Asp, Glu, Thr or Pro
  MOD_RES (20)..(20): Ser, Asp, Glu, Thr or Pro
  MOD_RES (25)..(25): Ser, Asp, Glu, Thr or Pro
  MOD_RES (30)..(30): Ser, Asp, Glu, Thr or Pro
  MOD_RES (35)..(35): Ser, Asp, Glu, Thr or Pro
  MOD_RES (40)..(40): Ser, Asp, Glu, Thr or Pro
  See specification as filed for detailed description of substitutions and preferred embodiments
GGGGXGGGGXGGGGXGGGGXGGGGXGGGGXGGGGXGGGGX

SEQ ID NO: 28 - protein, 315 aa, Artificial Sequence
  Description of Artificial Sequence Synthetic polypeptide
  MISC_FEATURE (1)..(20): This region may encompass 1-20 residues
  MOD_RES (21)..(21): Ser, Asp, Glu, Thr or Pro
  MISC_FEATURE (22)..(41): This region may encompass 1-20 residues
  MOD_RES (42)..(42): Ser, Asp, Glu, Thr or Pro
  MISC_FEATURE (43)..(62): This region may encompass 1-20 residues
  MOD_RES (63)..(63): Ser, Asp, Glu, Thr or Pro
  MISC_FEATURE (64)..(83): This region may encompass 1-20 residues
  MOD_RES (84)..(84): Ser, Asp, Glu, Thr or Pro
  MISC_FEATURE (85)..(104): This region may encompass 1-20 residues
  MOD_RES (105)..(105): Ser, Asp, Glu, Thr or Pro
  MISC_FEATURE (106)..(125): This region may encompass 1-20 residues
  MOD_RES (126)..(126): Ser, Asp, Glu, Thr or Pro
  MISC_FEATURE (127)..(146): This region may encompass 1-20 residues
  MOD_RES (147)..(147): Ser, Asp, Glu, Thr or Pro
  MISC_FEATURE (148)..(167): This region may encompass 1-20 residues
  MOD_RES (168)..(168): Ser, Asp, Glu, Thr or Pro
  MISC_FEATURE (169)..(188): This region may encompass 1-20 residues
  MOD_RES (189)..(189): Ser, Asp, Glu, Thr or Pro
  MISC_FEATURE (190)..(209): This region may encompass 1-20 residues
  MOD_RES (210)..(210): Ser, Asp, Glu, Thr or Pro
  MISC_FEATURE (211)..(230): This region may encompass 1-20 residues
  MOD_RES (231)..(231): Ser, Asp, Glu, Thr or Pro
  MISC_FEATURE (232)..(251): This region may encompass 1-20 residues
  MOD_RES (252)..(252): Ser, Asp, Glu, Thr or Pro
  MISC_FEATURE (253)..(272): This region may encompass 1-20 residues
  MOD_RES (273)..(273): Ser, Asp, Glu, Thr or Pro
  MISC_FEATURE (274)..(293): This region may encompass 1-20 residues
  MOD_RES (294)..(294): Ser, Asp, Glu, Thr or Pro
  MISC_FEATURE (295)..(314): This region may encompass 1-20 residues
  MOD_RES (315)..(315): Ser, Asp, Glu, Thr or Pro
  See specification as filed for detailed description of substitutions and preferred embodiments
GGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGXGGGGGGGGGGGGGGGGGGGGX

SEQ ID NO: 29 - protein, 8 aa, Artificial Sequence
  Description of Artificial Sequence Synthetic peptide
GPEGLRVG

SEQ ID NO: 30 - protein, 8 aa, Artificial Sequence
  Description of Artificial Sequence Synthetic peptide
YGAGLGVV

SEQ ID NO: 31 - protein, 8 aa, Artificial Sequence
  Description of Artificial Sequence Synthetic peptide
AGLGVVER

SEQ ID NO: 32 - protein, 8 aa, Artificial Sequence
  Description of Artificial Sequence Synthetic peptide
AGLGISST

SEQ ID NO: 33 - protein, 8 aa, Artificial Sequence
  Description of Artificial Sequence Synthetic peptide
EPQALAMS

SEQ ID NO: 34 - protein, 8 aa, Artificial Sequence
  Description of Artificial Sequence Synthetic peptide
QALAMSAI

SEQ ID NO: 35 - protein, 8 aa, Artificial Sequence
  Description of Artificial Sequence Synthetic peptide
AAYHLVSQ

SEQ ID NO: 36 - protein, 8 aa, Artificial Sequence
  Description of Artificial Sequence Synthetic peptide
MDAFLESS

SEQ ID NO: 37 - protein, 8 aa, Artificial Sequence
  Description of Artificial Sequence Synthetic peptide
ESLPVVAV

SEQ ID NO: 38 - protein, 8 aa, Artificial Sequence
  Description of Artificial Sequence Synthetic peptide
SAPAVESE

SEQ ID NO: 39 - protein, 8 aa, Artificial Sequence
  Description of Artificial Sequence Synthetic peptide
DVAQFVLT

SEQ ID NO: 40 - protein, 22 aa, Artificial Sequence
  Description of Artificial Sequence Synthetic peptide
GLLVAGVLVLLVSLGVAIHLCC

SEQ ID NO: 41 - protein, 25 aa, Artificial Sequence
  Description of Artificial Sequence Synthetic peptide
ALIVLGGVAGLLLFIGLGIFFCVRC

SEQ ID NO: 42 - protein, 23 aa, Artificial Sequence
  Description of Artificial Sequence Synthetic peptide
LCYLLDGILFIYGVILTALFLRV

SEQ ID NO: 43 - protein, 26 aa, Artificial Sequence
  Description of Artificial Sequence Synthetic peptide
WVLVVVGGVLACYSLLVTVAFIIFWV

SEQ ID NO: 44 - protein, 25 aa, Artificial Sequence
  Description of Artificial Sequence Synthetic peptide
AAILGLGLVLGLLGPLAILLALYLL

SEQ ID NO: 45 - protein, 24 aa, Artificial Sequence
  Description of Artificial Sequence Synthetic peptide
ALPAALAVISFLLGLGLGVACVLA

SEQ ID NO: 46 - protein, 218 aa, Artificial Sequence
  Description of Artificial Sequence Synthetic polypeptide
MQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 47 - protein, 260 aa, Artificial Sequence
  Description of Artificial Sequence Synthetic polypeptide
MKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 48 - protein, 295 aa, Artificial Sequence
  Description of Artificial Sequence Synthetic polypeptide
MEVDPIGHLYGSSGGSGGSGGSGGGSGGGSGGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 49 - protein, 295 aa, Artificial Sequence
  Description of Artificial Sequence Synthetic polypeptide
MEVDPIGHLYGGGGSSGGSGGSGLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 50 - protein, 294 aa, Artificial Sequence
  Description of Artificial Sequence Synthetic polypeptide
MGGVSCKDVYDEAFCWTGGGGSLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 51 - protein, 302 aa, Artificial Sequence
  Description of Artificial Sequence Synthetic polypeptide
MGGVSCKDVYDEAFCWTGGGGSSGGSGGSGLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 52 - protein, 253 aa, Artificial Sequence
  Description of Artificial Sequence Synthetic polypeptide
MGGVSCKDVYDEAFCWTGGGGSLSGRSDNHGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 53 - protein, 261 aa, Artificial Sequence
  Description of Artificial Sequence Synthetic polypeptide
MGGVSCKDVYDEAFCWTGGGGSSGGSGGSGLSGRSDNHGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 54 - protein, 218 aa, Artificial Sequence
  Description of Artificial Sequence Synthetic polypeptide
MQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 55 - protein, 260 aa, Artificial Sequence
  Description of Artificial Sequence Synthetic polypeptide
MKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGLNDIFEAQKIEWHE

SEQ ID NO: 56 - protein, 218 aa, Artificial Sequence
  Description of Artificial Sequence Synthetic polypeptide
MQEVTQIPAALSVPEGENLVLNCSFTDSAIYNLQWFRQDPGKGLTSLLLIQSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 57 - protein, 259 aa, Artificial Sequence
  Description of Artificial Sequence Synthetic polypeptide
KAGVTQTPRYLIKTRGQQVTLSCSPISGHRSVSWYQQTPGQGLQFLFEYTDMTLRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGLNDIFEAQKIEWHE

SEQ ID NO: 58 - protein, 259 aa, Artificial Sequence
  Description of Artificial Sequence Synthetic polypeptide
KAGVTQTPRYLIKTRGQQVTLSCSPISGHRSVSWYQQTPGQGLQFLFEYFDMLLRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGLNDIFEAQKIEWHE

SEQ ID NO: 59 - protein, 5 aa, Artificial Sequence
  Description of Artificial Sequence Synthetic peptide
  MOD_RES (3)..(4): Any amino acid
YDXXF

SEQ ID NO: 60 - protein, 7 aa, Artificial Sequence
  Description of Artificial Sequence Synthetic peptide
DVYDEAF

SEQ ID NO: 61 - protein, 16 aa, Artificial Sequence
  Description of Artificial Sequence Synthetic peptide
GGVSCKDVYDEAFCWT

SEQ ID NO: 62 - protein, 18 aa, Artificial Sequence
  Description of Artificial Sequence Synthetic peptide
GGGGSLSGRSDNHGSSGT

SEQ ID NO: 63 - protein, 26 aa, Artificial Sequence
  Description of Artificial Sequence Synthetic peptide
GGGGSSGGSGGSGLSGRSDNHGSSGT

SEQ ID NO: 64 - protein, 9 aa, Artificial Sequence
  Description of Artificial Sequence Synthetic peptide
  MISC_FEATURE (1)..(9): This sequence may encompass 1-3 “Gly Gly Ser” repeating units
GGSGGSGGS

SEQ ID NO: 65 - protein, 12 aa, Artificial Sequence
  Description of Artificial Sequence Synthetic peptide
LVWGCIWDDMCS

SEQ ID NO: 66 - protein, 12 aa, Artificial Sequence
  Description of Artificial Sequence Synthetic peptide
WHWEPSMVWGML

SEQ ID NO: 67 - protein, 12 aa, Artificial Sequence
  Description of Artificial Sequence Synthetic peptide
VRTWFEKFPELV

SEQ ID NO: 68 - protein, 16 aa, Artificial Sequence
  Description of Artificial Sequence Synthetic peptide
GGNACEMTYDHTFCDP

SEQ ID NO: 69 - protein, 16 aa, Artificial Sequence
  Description of Artificial Sequence Synthetic peptide
GGIICWFDNGLVQCSW

SEQ ID NO: 70 - protein, 16 aa, Artificial Sequence
  Description of Artificial Sequence Synthetic peptide
GGDLCDSAWAYWYCDE

SEQ ID NO: 71 - protein, 16 aa, Artificial Sequence
  Description of Artificial Sequence Synthetic peptide
GGESCQSVYDSSFCYD

SEQ ID NO: 72 - protein, 16 aa, Artificial Sequence
  Description of Artificial Sequence Synthetic peptide
GGCSWIFDGLRYFSKC

SEQ ID NO: 73 - protein, 16 aa, Artificial Sequence
  Description of Artificial Sequence Synthetic peptide
GGGMCSLVYDSVFCDQ

SEQ ID NO: 74 - protein, 16 aa, Artificial Sequence
  Description of Artificial Sequence Synthetic peptide
GGCELYYSWSGSYDMC

SEQ ID NO: 75 - protein, 16 aa, Artificial Sequence
  Description of Artificial Sequence Synthetic peptide
GGDCQPDSVWSYWYCR

SEQ ID NO: 76 - protein, 16 aa, Artificial Sequence
  Description of Artificial Sequence Synthetic peptide
GGCTFVDWWVLGSPYC

SEQ ID NO: 77 - protein, 16 aa, Artificial Sequence
  Description of Artificial Sequence Synthetic peptide
GGVACREVYDHYFCWD

SEQ ID NO: 78 - protein, 16 aa, Artificial Sequence
  Description of Artificial Sequence Synthetic peptide
GGVSCKDVYDEAFCWT

SEQ ID NO: 79 - protein, 16 aa, Artificial Sequence
  Description of Artificial Sequence Synthetic peptide
GGNSCSLVYDKAFCLF

SEQ ID NO: 80 - protein, 16 aa, Artificial Sequence
  Description of Artificial Sequence Synthetic peptide
GGRRCVDVYDNAFCLI

SEQ ID NO: 81 - protein, 16 aa, Artificial Sequence
  Description of Artificial Sequence Synthetic peptide
GGRACSDIYDAEFCGL

SEQ ID NO: 82 - protein, 16 aa, Artificial Sequence
  Description of Artificial Sequence Synthetic peptide
GGCLMNDYYYLWGGHC

SEQ ID NO: 83 - protein, 16 aa, Artificial Sequence
  Description of Artificial Sequence Synthetic peptide
GGTSCAQIYDFEFCYS

SEQ ID NO: 84 - protein, 16 aa, Artificial Sequence
  Description of Artificial Sequence Synthetic peptide
GGSLCSLVYDQDFCES

SEQ ID NO: 85 - protein, 16 aa, Artificial Sequence
  Description of Artificial Sequence Synthetic peptide
GGVPCWMVYDALFCGL

SEQ ID NO: 86 - protein, 16 aa, Artificial Sequence
  Description of Artificial Sequence Synthetic peptide
GGGCFVSPATGFTWCV

SEQ ID NO: 87 - protein, 16 aa, Artificial Sequence
  Description of Artificial Sequence Synthetic peptide
GGNLCHDVYDMSFCYG

SEQ ID NO: 88 - protein, 16 aa, Artificial Sequence
  Description of Artificial Sequence Synthetic peptide
GGTFCYFDHGLVNCQW

SEQ ID NO: 89 - protein, 16 aa, Artificial Sequence
  Description of Artificial Sequence Synthetic peptide
GGRICEEVYDWIFCES

SEQ ID NO: 90 - protein, 16 aa, Artificial Sequence
  Description of Artificial Sequence Synthetic peptide
GGSACTRVYDYDFCYG

SEQ ID NO: 91 - protein, 16 aa, Artificial Sequence
  Description of Artificial Sequence Synthetic peptide
GGNQCWEVYDQEFCSL

SEQ ID NO: 92 - protein, 16 aa, Artificial Sequence
  Description of Artificial Sequence Synthetic peptide
GGERCESVYDLFFCYG

SEQ ID NO: 93 - protein, 16 aa, Artificial Sequence
  Description of Artificial Sequence Synthetic peptide
GGVLCETVYDRDFCFA

SEQ ID NO: 94 - protein, 16 aa, Artificial Sequence
  Description of Artificial Sequence Synthetic peptide
GGGNCSVIYDDLFCLV

SEQ ID NO: 95 - protein, 16 aa, Artificial Sequence
  Description of Artificial Sequence Synthetic peptide
GGSACSRIYDFAFCHT

SEQ ID NO: 96 - protein, 16 aa, Artificial Sequence
  Description of Artificial Sequence Synthetic peptide
GGHCFVSPASGEWWCV

SEQ ID NO: 97 - protein, 14 aa, Artificial Sequence
  Description of Artificial Sequence Synthetic peptide
GGLCDLSGLWPLYC

SEQ ID NO: 98 - protein, 16 aa, Artificial Sequence
  Description of Artificial Sequence Synthetic peptide
GGCVFYGSVDHIWYDC

SEQ ID NO: 99 - protein, 16 aa, Artificial Sequence
  Description of Artificial Sequence Synthetic peptide
GGGYCSIVYDRLFCSS

SEQ ID NO: 100 - protein, 16 aa, Artificial Sequence
  Description of Artificial Sequence Synthetic peptide
GGQECHTVYDVQFCSH

SEQ ID NO: 101 - protein, 16 aa, Artificial Sequence
  Description of Artificial Sequence Synthetic peptide
GGCGFNIAAPLYGLVC

SEQ ID NO: 102 - protein, 16 aa, Artificial Sequence
  Description of Artificial Sequence Synthetic peptide
GGQECSFIYDRVFCLV

SEQ ID NO: 103 - protein, 16 aa, Artificial Sequence
  Description of Artificial Sequence Synthetic peptide
GGGCSYQGPWEFWYCR

SEQ ID NO: 104 - protein, 16 aa, Artificial Sequence
  Description of Artificial Sequence Synthetic peptide
GGLHGCFDGTFVSCSW

SEQ ID NO: 105 - protein, 16 aa, Artificial Sequence
  Description of Artificial Sequence Synthetic peptide
GGLADYCEHPMCYWYS

SEQ ID NO: 106 - protein, 16 aa, Artificial Sequence
  Description of Artificial Sequence Synthetic peptide
GGPCVSVLQELVLEWC

SEQ ID NO: 107 - protein, 16 aa, Artificial Sequence
  Description of Artificial Sequence Synthetic peptide
GGSTWGCIWDDMCGQA

SEQ ID NO: 108 - protein, 16 aa, Artificial Sequence
  Description of Artificial Sequence Synthetic peptide
GGACVAENEWAYWYCR

SEQ ID NO: 109 - protein, 16 aa, Artificial Sequence
  Description of Artificial Sequence Synthetic peptide
GGFTFTCMLHDCVYIL

SEQ ID NO: 110 - protein, 16 aa, Artificial Sequence
  Description of Artificial Sequence Synthetic peptide
GGHHCTQVYDYSFCFM

SEQ ID NO: 111 - protein, 16 aa, Artificial Sequence
  Description of Artificial Sequence Synthetic peptide
GGQICADTYDWVFCFE

SEQ ID NO: 112 - protein, 16 aa, Artificial Sequence
  Description of Artificial Sequence Synthetic peptide
GGAHCHQVYDYSFCFL

SEQ ID NO: 113 - protein, 16 aa, Artificial Sequence
  Description of Artificial Sequence Synthetic peptide
GGSFCEIVYDALFCDM

SEQ ID NO: 114 - protein, 16 aa, Artificial Sequence
  Description of Artificial Sequence Synthetic peptide
GGLECSLTYDWEFCKY

SEQ ID NO: 115 - protein, 16 aa, Artificial Sequence
  Description of Artificial Sequence Synthetic peptide
GGFIFSCSNDECFYFL

SEQ ID NO: 116 - protein, 16 aa, Artificial Sequence
  Description of Artificial Sequence Synthetic peptide
GGYECRRSLDADICWI

SEQ ID NO: 117 - protein, 16 aa, Artificial Sequence
  Description of Artificial Sequence Synthetic peptide
GGWCSVWFFDGWEWCG

SEQ ID NO: 118 - protein, 16 aa, Artificial Sequence
  Description of Artificial Sequence Synthetic peptide
GGSFCYTHPFGYFYCR

SEQ ID NO: 119 - protein, 16 aa, Artificial Sequence
  Description of Artificial Sequence Synthetic peptide
GGSLCGDGYGWYWMCL

SEQ ID NO: 120 - protein, 16 aa, Artificial Sequence
  Description of Artificial Sequence Synthetic peptide
GGMWFCDWKWDSLCDV

SEQ ID NO: 121 - protein, 16 aa, Artificial Sequence
  Description of Artificial Sequence Synthetic peptide
GGPVFCFDGTVFGCWL

SEQ ID NO: 122 - protein, 16 aa, Artificial Sequence
  Description of Artificial Sequence Synthetic peptide
GGIRTCWHPFVVWCMS

SEQ ID NO: 123 - protein, 16 aa, Artificial Sequence
  Description of Artificial Sequence Synthetic peptide
GGFCQALRAEYYFFCS

SEQ ID NO: 124 - protein, 16 aa, Artificial Sequence
  Description of Artificial Sequence Synthetic peptide
GGTPFCFDGMVYACRS

SEQ ID NO: 125 - protein, 16 aa, Artificial Sequence
  Description of Artificial Sequence Synthetic peptide
GGEWFCDWAWGVYCKA

SEQ ID NO: 126 - protein, 16 aa, Artificial Sequence
  Description of Artificial Sequence Synthetic peptide
GGGLCWHPFVPHYYCR

SEQ ID NO: 127 - protein, 16 aa, Artificial Sequence
  Description of Artificial Sequence Synthetic peptide
GGHECVMVYGEWEFCN

SEQ ID NO: 128 - protein, 16 aa, Artificial Sequence
  Description of Artificial Sequence Synthetic peptide
GGSWFCDHMWLEYCGS

SEQ ID NO: 129 - protein, 16 aa, Artificial Sequence
  Description of Artificial Sequence Synthetic peptide
GGMVCWDGPWHFVCPG

SEQ ID NO: 130 - protein, 16 aa, Artificial Sequence
  Description of Artificial Sequence Synthetic peptide
GGGFCEDGMRWTQCIF

SEQ ID NO: 131 - protein, 16 aa, Artificial Sequence
  Description of Artificial Sequence Synthetic peptide
GGYACWFGHGLVHCGT

SEQ ID NO: 132 - protein, 16 aa, Artificial Sequence
  Description of Artificial Sequence Synthetic peptide
GGSEIRCDWFWCFDVL

SEQ ID NO: 133 - protein, 16 aa, Artificial Sequence
  Description of Artificial Sequence Synthetic peptide
GGCTFVEWWHHGYKLC

SEQ ID NO: 134 - protein, 16 aa, Artificial Sequence
  Description of Artificial Sequence Synthetic peptide
GGRVCYFDHQVVHCIW

SEQ ID NO: 135 - protein, 16 aa, Artificial Sequence
  Description of Artificial Sequence Synthetic peptide
GGLWCHEIYDLAFCRF

SEQ ID NO: 136 - protein, 16 aa, Artificial Sequence
  Description of Artificial Sequence Synthetic peptide
GGCEWLPGLVHLIYHC

SEQ ID NO: 137 - protein, 16 aa, Artificial Sequence
  Description of Artificial Sequence Synthetic peptide
GGYPCHQIYDSNFCYF

SEQ ID NO: 138 - protein, 16 aa, Artificial Sequence
  Description of Artificial Sequence Synthetic peptide
GGCQVEWWGKPYTINC

SEQ ID NO: 139 - protein, 16 aa, Artificial Sequence
  Description of Artificial Sequence Synthetic peptide
GGEPCHEVFDHSFCMM

SEQ ID NO: 140 - protein, 16 aa, Artificial Sequence
  Description of Artificial Sequence Synthetic peptide
GGHCIEMIYGWMGYTC

SEQ ID NO: 141 - protein, 16 aa, Artificial Sequence
  Description of Artificial Sequence Synthetic peptide
GGMCVDRTNWLWQIYC

SEQ ID NO: 142 - protein, 16 aa, Artificial Sequence
  Description of Artificial Sequence Synthetic peptide
GGSCWDVYGKWAYWHC

SEQ ID NO: 143 - protein, 16 aa, Artificial Sequence
  Description of Artificial Sequence Synthetic peptide
GGYCHPFLAWQHDYFC

SEQ ID NO: 144 - protein, 16 aa, Artificial Sequence
  Description of Artificial Sequence Synthetic peptide
GGICREFSGEWWVWDC

SEQ ID NO: 145 - protein, 16 aa, Artificial Sequence
  Description of Artificial Sequence Synthetic peptide
GGDPACSPSTLVCWLF

SEQ ID NO: 146 - protein, 16 aa, Artificial Sequence
  Description of Artificial Sequence Synthetic peptide
GGYVTCHWDQSFCWFH

SEQ ID NO: 147 - protein, 16 aa, Artificial Sequence
  Description of Artificial Sequence Synthetic peptide
GGIVYCFFDSPWCFVR

SEQ ID NO: 148 - protein, 16 aa, Artificial Sequence
  Description of Artificial Sequence Synthetic peptide
GGVPDWCWTIGLCFGT

SEQ ID NO: 149 - protein, 16 aa, Artificial Sequence
  Description of Artificial Sequence Synthetic peptide
GGAVYCFDGWFFTCGG

SEQ ID NO: 150 - protein, 16 aa, Artificial Sequence
  Description of Artificial Sequence Synthetic peptide
GGQSPCFDGTVIACLG

SEQ ID NO: 151 - protein, 16 aa, Artificial Sequence
  Description of Artificial Sequence Synthetic peptide
GGSMCEHVYDWLFCFV

SEQ ID NO: 152 - protein, 16 aa, Artificial Sequence
  Description of Artificial Sequence Synthetic peptide
GGHFCSRVYDESFCDE

SEQ ID NO: 153 - protein, 16 aa, Artificial Sequence
  Description of Artificial Sequence Synthetic peptide
GGVICWFQEGMVHCVS

SEQ ID NO: 154 - protein, 16 aa, Artificial Sequence
  Description of Artificial Sequence Synthetic peptide
GGQHCNDVYDWAFCLI

SEQ ID NO: 155 - protein, 16 aa, Artificial Sequence
  Description of Artificial Sequence Synthetic peptide
GGVLCFVHQNQTFECS

SEQ ID NO: 156 - protein, 16 aa, Artificial Sequence
  Description of Artificial Sequence Synthetic peptide
GGGCYVHPGSGVFWCS

SEQ ID NO: 157 - protein, 16 aa, Artificial Sequence
  Description of Artificial Sequence Synthetic peptide
GGTVHCFDGVVFSCLG

SEQ ID NO: 158 - protein, 16 aa, Artificial Sequence
  Description of Artificial Sequence Synthetic peptide
GGHCYALTFMDYWACN

SEQ ID NO: 159 - protein, 16 aa, Artificial Sequence
  Description of Artificial Sequence Synthetic peptide
GGMMCEHVYDFLFCLS

SEQ ID NO: 160 - protein, 16 aa, Artificial Sequence
  Description of Artificial Sequence Synthetic peptide
GGSMIKCNFPRLCNGK

SEQ ID NO: 161 - protein, 16 aa, Artificial Sequence
  Description of Artificial Sequence Synthetic peptide
GGFCEQGQFMAAWHRC

SEQ ID NO: 162 - protein, 16 aa, Artificial Sequence
  Description of Artificial Sequence Synthetic peptide
GGDLLQCPQAVSCRPR

SEQ ID NO: 163 - protein, 16 aa, Artificial Sequence
  Description of Artificial Sequence Synthetic peptide
GGDVRCFMRMMECVLL

SEQ ID NO: 164 - protein, 21 aa, Artificial Sequence
  Description of Artificial Sequence Synthetic peptide
GGHCVDMVDFYQQTCQGGGGS

SEQ ID NO: 165 - protein, 16 aa, Artificial Sequence
  Description of Artificial Sequence Synthetic peptide
GGIWCWFENQSVICTA

SEQ ID NO: 166 - protein, 16 aa, Artificial Sequence
  Description of Artificial Sequence Synthetic peptide
GGIICWFQSFEVYCMG

SEQ ID NO: 167 - protein, 16 aa, Artificial Sequence
  Description of Artificial Sequence Synthetic peptide
GGIICWFEVGQVRCQD

SEQ ID NO: 168 - protein, 16 aa, Artificial Sequence
  Description of Artificial Sequence Synthetic peptide
GGCFSYSPWGTTWSHC

SEQ ID NO: 169 - protein, 16 aa, Artificial Sequence
  Description of Artificial Sequence Synthetic peptide
GGVCFVLPWPQLKLVC

SEQ ID NO: 170 - protein, 16 aa, Artificial Sequence
  Description of Artificial Sequence Synthetic peptide
GGRGCYFDHQVVHCIW

SEQ ID NO: 171 - protein, 16 aa, Artificial Sequence
  Description of Artificial Sequence Synthetic peptide
GGYLCWFEGGGLVSCA

SEQ ID NO: 172 - protein, 16 aa, Artificial Sequence
  Description of Artificial Sequence Synthetic peptide
GGSVCFFKGGGLVICY

SEQ ID NO: 173 - protein, 16 aa, Artificial Sequence
  Description of Artificial Sequence Synthetic peptide
GGLSCWFAGYARVECA

SEQ ID NO: 174 - protein, 16 aa, Artificial Sequence
  Description of Artificial Sequence Synthetic peptide
GGVECWFAGGGDVICA

SEQ ID NO: 175 - protein, 16 aa, Artificial Sequence
  Description of Artificial Sequence Synthetic peptide
GGRFCWFAAFSTVLCV

SEQ ID NO: 176 - protein, 16 aa, Artificial Sequence
  Description of Artificial Sequence Synthetic peptide
GGITCFFYPAHMVTCS

SEQ ID NO: 177 - protein, 16 aa, Artificial Sequence
  Description of Artificial Sequence Synthetic peptide
GGYFCWFSGEKAVICS

SEQ ID NO: 178 - protein, 16 aa, Artificial Sequence
  Description of Artificial Sequence Synthetic peptide
GGMVCFFEGRGQVVCI

SEQ ID NO: 179 - protein, 16 aa, Artificial Sequence
  Description of Artificial Sequence Synthetic peptide
GGIFCWFVGSSTVTCE

SEQ ID NO: 180 - protein, 16 aa, Artificial Sequence
  Description of Artificial Sequence Synthetic peptide
GGYGCLGGLWDYWYCA

SEQ ID NO: 181 - protein, 16 aa, Artificial Sequence
  Description of Artificial Sequence Synthetic peptide
GGYLCFFEGGGLVSCA

SEQ ID NO: 182 - protein, 16 aa, Artificial Sequence
  Description of Artificial Sequence Synthetic peptide
GGISCWFSGVGQVLCY

SEQ ID NO: 183 - protein, 16 aa, Artificial Sequence
  Description of Artificial Sequence Synthetic peptide
GGYLCCFEGGGLVSCA

SEQ ID NO: 184 - protein, 16 aa, Artificial Sequence
  Description of Artificial Sequence Synthetic peptide
GGVICENWQGDRVCWF

SEQ ID NO: 185 - protein, 16 aa, Artificial Sequence
  Description of Artificial Sequence Synthetic peptide
GGYFCYFESSMSHCLY

SEQ ID NO: 186 - protein, 16 aa, Artificial Sequence
  Description of Artificial Sequence Synthetic peptide
GGIFCWFQDFSVYCKS

SEQ ID NO: 187 - protein, 16 aa, Artificial Sequence
  Description of Artificial Sequence Synthetic peptide
GGYVCYFYNASVTCVY

SEQ ID NO: 188 - protein, 16 aa, Artificial Sequence
  Description of Artificial Sequence Synthetic peptide
GGQMCHFEYNLVVCYH

SEQ ID NO: 189 - protein, 16 aa, Artificial Sequence
  Description of Artificial Sequence Synthetic peptide
GGCYFDFGVLGTSVVC

SEQ ID NO: 190 - protein, 16 aa, Artificial Sequence
  Description of Artificial Sequence Synthetic peptide
GGRMCHFDVNTVVCYL

SEQ ID NO: 191 - protein, 16 aa, Artificial Sequence
  Description of Artificial Sequence Synthetic peptide
GGSVCYFELSVVICVN

SEQ ID NO: 192 - protein, 16 aa, Artificial Sequence
  Description of Artificial Sequence Synthetic peptide
GGRTCYFDQGSVVCYW

SEQ ID NO: 193 - protein, 16 aa, Artificial Sequence
  Description of Artificial Sequence Synthetic peptide
GGVFCWFEWSVVTCSH

SEQ ID NO: 194 - protein, 16 aa, Artificial Sequence
  Description of Artificial Sequence Synthetic peptide
GGYMCYFSMKTVVCQW

SEQ ID NO: 195 - protein, 16 aa, Artificial Sequence
  Description of Artificial Sequence Synthetic peptide
GGDFCWFFNREVLCYG

SEQ ID NO: 196 - protein, 16 aa, Artificial Sequence
  Description of Artificial Sequence Synthetic peptide
GGTFCYFVNFSVTCVN

SEQ ID NO: 197 - protein, 16 aa, Artificial Sequence
  Description of Artificial Sequence Synthetic peptide
GGSVNCIDAIFACFLV

SEQ ID NO: 198 - protein, 21 aa, Artificial Sequence
  Description of Artificial Sequence Synthetic peptide
GGESCQSVYDSSFCYDGGGGS

SEQ ID NO: 199 - protein, 21 aa, Artificial Sequence
  Description of Artificial Sequence Synthetic peptide
GGNACEMTYDHTFCDPGGGGS

SEQ ID NO: 200 - protein, 21 aa, Artificial Sequence
  Description of Artificial Sequence Synthetic peptide
GGRICEEVYDWIFCDPGGGGS

SEQ ID NO: 201 - protein, 21 aa, Artificial Sequence
  Description of Artificial Sequence Synthetic peptide
GGRRCVDVYDNAFCLIGGGGS

SEQ ID NO: 202 - protein, 21 aa, Artificial Sequence
  Description of Artificial Sequence Synthetic peptide
GGVSCKDVYDEAFCWTGGGGS

SEQ ID NO: 203 - protein, 21 aa, Artificial Sequence
  Description of Artificial Sequence Synthetic peptide
GGTSCAQIYDFEFCYSGGGGS

SEQ ID NO: 204 - protein, 21 aa, Artificial Sequence
  Description of Artificial Sequence Synthetic peptide
GGSLCSLVYDQDFCESGGGGS

SEQ ID NO: 205 - protein, 21 aa, Artificial Sequence
  Description of Artificial Sequence Synthetic peptide
GGNSCSLVYDKAFCLFGGGGS

SEQ ID NO: 206 - protein, 21 aa, Artificial Sequence
  Description of Artificial Sequence Synthetic peptide
GGNQCWEVYDQEFCSLGGGGS

SEQ ID NO: 207 - protein, 21 aa, Artificial Sequence
  Description of Artificial Sequence Synthetic peptide
GGSACSRIYDFAFCHTGGGGS

SEQ ID NO: 208 - protein, 21 aa, Artificial Sequence
  Description of Artificial Sequence Synthetic peptide
GGTFCYFDHGLVNCQWGGGGS

SEQ ID NO: 209 - protein, 21 aa, Artificial Sequence
  Description of Artificial Sequence Synthetic peptide
GGHCFVSPASGEWWCVGGGGS

SEQ ID NO: 210 - protein, 21 aa, Artificial Sequence
  Description of Artificial Sequence Synthetic peptide
GGCSWIFDGLRYFSKCGGGGS

SEQ ID NO: 211 - protein, 17 aa, Artificial Sequence
  Description of Artificial Sequence Synthetic peptide
VRTWFEKFPELVGGGGS

SEQ ID NO: 212 - protein, 17 aa, Artificial Sequence
  Description of Artificial Sequence Synthetic peptide
LVWGCIWDDMCSGGGGS

SEQ ID NO: 213 - protein, 17 aa, Artificial Sequence
  Description of Artificial Sequence Synthetic peptide
WHWEPSMVWGMLGGGGS

SEQ ID NO: 214 - protein, 21 aa, Artificial Sequence
  Description of Artificial Sequence Synthetic peptide
GGGCFVSPATGFTWCVGGGGS

SEQ ID NO: 215 - protein, 21 aa, Artificial Sequence
  Description of Artificial Sequence Synthetic peptide
GGDCQPDSVWSYWYCRGGGGS

SEQ ID NO: 216 - protein, 21 aa, Artificial Sequence
  Description of Artificial Sequence Synthetic peptide
GGCTFVDWWVLGSPYCGGGGS

SEQ ID NO: 217 - protein, 21 aa, Artificial Sequence
  Description of Artificial Sequence Synthetic peptide
GGCLMNDYYYLWGGHCGGGGS

SEQ ID NO: 218 - protein, 21 aa, Artificial Sequence
  Description of Artificial Sequence Synthetic peptide
GGASCKDVYDEAFCWTGGGGS

SEQ ID NO: 219 - protein, 21 aa, Artificial Sequence
  Description of Artificial Sequence Synthetic peptide
GGVACKDVYDEAFCWTGGGGS

SEQ ID NO: 220 - protein, 21 aa, Artificial Sequence
  Description of Artificial Sequence Synthetic peptide
GGVSAKDVYDEAFCWTGGGGS

SEQ ID NO: 221 - protein, 21 aa, Artificial Sequence
  Description of Artificial Sequence Synthetic peptide
GGVSCADVYDEAFCWTGGGGS

SEQ ID NO: 222 - protein, 21 aa, Artificial Sequence
  Description of Artificial Sequence Synthetic peptide
GGVSCKAVYDEAFCWTGGGGS

SEQ ID NO: 223 - protein, 21 aa, Artificial Sequence
  Description of Artificial Sequence Synthetic peptide
GGVSCKDAYDEAFCWTGGGGS

SEQ ID NO: 224 - protein, 21 aa, Artificial Sequence
  Description of Artificial Sequence Synthetic peptide
GGVSCKDVADEAFCWTGGGGS

SEQ ID NO: 225 - protein, 21 aa, Artificial Sequence
  Description of Artificial Sequence Synthetic peptide
GGVSCKDVYAEAFCWTGGGGS

SEQ ID NO: 226 - protein, 21 aa, Artificial Sequence
  Description of Artificial Sequence Synthetic peptide
GGVSCKDVYDAAFCWTGGGGS

SEQ ID NO: 227 - protein, 21 aa, Artificial Sequence
  Description of Artificial Sequence Synthetic peptide
GGVSCKDVYDEAACWTGGGGS

SEQ ID NO: 228 - protein, 21 aa, Artificial Sequence
  Description of Artificial Sequence Synthetic peptide
GGVSCKDVYDEAFAWTGGGGS

SEQ ID NO: 229 - protein, 21 aa, Artificial Sequence
  Description of Artificial Sequence Synthetic peptide
GGVSCKDVYDEAFCATGGGGS

SEQ ID NO: 230 - protein, 21 aa, Artificial Sequence
  Description of Artificial Sequence Synthetic peptide
GGVSCKDVYDEAFCWAGGGGS

SEQ ID NO: 231 - protein, 14 aa, Artificial Sequence
  Description of Artificial Sequence Synthetic peptide
EVDPIGHLYGGGGS

SEQ ID NO: 232 - protein, 14 aa, Artificial Sequence
  Description of Artificial Sequence Synthetic peptide
ESDPIVAQYGGGGS

SEQ ID NO: 233 - protein, 21 aa, Artificial Sequence
  Description of Artificial Sequence Synthetic peptide
GGGGSGGVSCKDVYDEAFCWT

SEQ ID NO: 234 - protein, 19 aa, Artificial Sequence
  Description of Artificial Sequence Synthetic peptide
GGHIAHVYCEHVMCGGGGS

SEQ ID NO: 235 - protein, 19 aa, Artificial Sequence
  Description of Artificial Sequence Synthetic peptide
GGWPESGHPWWYSAGGGGS

SEQ ID NO: 236 - protein, 19 aa, Artificial Sequence
  Description of Artificial Sequence Synthetic peptide
GGVHSYCEHIMCGFGGGGS

(19) United States
(12) Patent Application Publication
(10) Pub. No.: US 2021/0115106 A1
(43) Pub. Date: Apr. 22, 2021

(54) MODIFIED T CELL RECEPTORS

(71) Applicant: JANUX THERAPEUTICS, INC., La Jolla, CA (US)

(72) Inventors: David CAMPBELL, La Jolla, CA (US); Ramesh BHATT, La Jolla, CA (US); Thomas R. DIRAIMONDO, La Jolla, CA (US); Steven E. CWIRLA, Menlo Park, CA (US); Blake M. WILLIAMS, Menlo Park, CA (US); Praechompoo PONGTORNPIPAT, Menlo Park, CA (US); William J. DOWER, Menlo Park, CA (US)

(21) Appl. No.: 16/769,536

(22) PCT Filed: Dec. 6, 2018
(86) PCT No.: PCT/US2018/064347

§ 371 Date: Jun. 3, 2020

Related U.S. Application Data
(60) Provisional application No. 62/595,976, filed on Dec. 7, 2017.

(51) Int. Cl. C07K 14/725; C07K 16/28

(52) U.S. Cl. CPC C07K 14/7051; C07K 2317/565; C07K 16/28

(57) ABSTRACT

Provided herein are modified T cell receptors (TCRs), pharmaceutical compositions thereof, as well as nucleic acids, and methods for making and discovering the same. The modified TCRs described herein are modified with a peptide.

### CROSS-REFERENCE

This application claims the benefit of U.S. Provisional Application No. 62/595,976 filed Dec. 7, 2017, which is incorporated by reference herein in its entirety.

### BACKGROUND OF THE INVENTION

Protein-based therapies, such as modified T cell receptors (TCRs), have proven effective as treatments for a variety of diseases. As with any therapeutic class, there is a need to improve toxicity and side effects of such treatments.

### REFERENCE TO A SEQUENCE LISTING

The instant application contains a Sequence Listing which has been filed electronically in ASCII format and is hereby incorporated by reference in its entirety. Said ASCII copy, created on Dec. 6, 2018, is named 52426-705_601_SL.txt and is 108,255 bytes in size.

### SUMMARY OF THE INVENTION

Disclosed herein, in certain embodiments, are modified T cell receptors (TCRs) comprising a polypeptide of formula III: T₃-L₃-P₃ (formula III) wherein: T₃ comprises either a TCR alpha extracellular domain, or a fragment thereof, or a TCR beta extracellular domain, or a fragment thereof, wherein T₃ binds to a target antigen, and the TCR alpha extracellular domain or fragment thereof and the TCR beta extracellular domain, or fragment thereof contain an antigen binding site; P₃ is a peptide that reduces binding of T₃ to the target antigen when the modified TCR is outside of a tumor microenvironment and that does not reduce binding of T₃ to the target antigen when the modified TCR is inside the tumor microenvironment; and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease. In some instances, P₃ is bound to T₃ through ionic interactions, electrostatic interactions, hydrophobic interactions, Pi-stacking interactions, and H-bonding interactions, or a combination thereof when the modified TCR is outside the tumor microenvironment. In some instances, P₃ is bound to T₃ at or near the antigen binding site when the modified TCR is outside the tumor microenvironment. In some instances, P₃ inhibits the binding of T₃ to the target antigen when the modified TCR is outside the tumor microenvironment, and P₃ does not inhibit the binding of T₃ to the target antigen when the modified TCR is inside the tumor microenvironment. In some instances, P₃ sterically blocks T₃ from binding to the target antigen when the modified TCR is outside the tumor microenvironment. In some instances, P₃ is removed from the antigen binding site, and the antigen binding site of T₃ is exposed when the modified TCR is inside the tumor microenvironment. In some instances, P₃ comprises less than 70% sequence homology to the target antigen. In some instances, P₃ comprises a peptide sequence of at least 10 amino acids in length. In some instances, P₃ comprises a peptide sequence of at least 10 amino acids in length and no more than 20 amino acids in length. In some instances, P₃ comprises a peptide sequence of at least 16 amino acids in length. In some instances, P₃ comprises at least two cysteine amino acid residues. In some instances, P₃ comprises an amino acid sequence according to SEQ ID NO: 59 (YDXXF), wherein X is any amino acid. In some instances, P₃ comprises an amino acid sequence according to SEQ ID NO: 59 (YDXXF), wherein X is any amino acid except for cysteine. In some instances, P₃ comprises an amino acid sequence according to SEQ ID NO: 60 (DVYDEAF). In some instances, P₃ comprises an amino sequence according to SEQ ID NO: 61 (GGVSCKDVYDEAFCWT) (Peptide-5). In some instances, P₃ comprises a cyclic peptide or a linear peptide. In some instances, P₃ comprises a cyclic peptide. In some instances, P₃ comprises a linear peptide. In some instances, L₃ is a peptide sequence having at least 5 to no more than 50 amino acids. In some instances, L₃ is a peptide sequence having at least 10 to no more than 30 amino acids. In some instances, L₃ is a peptide sequence having at least 10 amino acids. In some instances, L₃ is a peptide sequence having at least 18 amino acids. In some instances, L₃ is a peptide sequence having at least 26 amino acids. In some instances, L₃ has a formula comprising (G₂S)ₙ, wherein n is an integer from 1 to 3 (SEQ ID NO: 64). In some instances, L₃ is a substrate for a tumor specific protease. In some instances, the tumor specific protease is selected from the group consisting of metalloprotease, serine protease, cysteine protease, threonine protease, and aspartic protease. In some instances, L₃ comprises a urokinase cleavable amino acid sequence, a MT-SP1 cleavable amino acid sequence, or a KLK5 cleavable amino acid sequence. In some instances, L₃ comprises an amino acid sequence according to SEQ ID NO: 62 (GGGGSLSGRSDNHGSSGT). In some instances, L₃ comprises an amino acid sequence according to SEQ ID NO: 63 (GGGGSSGGSGGSGLSGRSDNHGSSGT). In some instances, T₃ comprises a MAGE-A3 domain. In some instances, T₃ comprises a MAGE-A3 alpha domain. In some instances, T₃ comprises a MAGE-A3 beta domain. In some instances, T₃ comprises an amino acid sequence according to SEQ ID NO: 46. In some instances, T₃ comprises an amino acid sequence according to SEQ ID NO: 47. In some instances, T₃ comprises an amino acid sequence according to SEQ ID NO: 54. In some instances, T₃ comprises an amino acid sequence according to SEQ ID NO: 55. In some instances, T₃ comprises the TCR alpha extracellular domain, or fragment thereof, and the modified TCR further comprises a second polypeptide comprising a TCR beta extracellular domain, or a fragment thereof wherein the TCR beta extracellular domain or fragment thereof contains an antigen binding site. In some instances, T₃ comprises the TCR beta extracellular domain, or fragment thereof, and the modified TCR further comprises a second polypeptide comprising a TCR alpha extracellular domain, or a fragment thereof wherein the TCR alpha extracellular domain or fragment thereof contains an antigen binding site. In some instances, the TCR alpha extracellular domain, or fragment thereof, comprises three hypervariable complementarity determining regions (CDRs). In some instances, at least one CDR comprises a mutation to increase binding affinity or binding specificity to the target antigen or to increase binding affinity and binding specificity to the target antigen. In some instances, the TCR beta extracellular domain, or fragment thereof, comprises three hypervariable complementarity determining regions (CDRs). In some instances, at least one CDR comprises a mutation to increase binding affinity or binding specificity to the target antigen or to increase binding affinity and binding specificity to the target antigen. In some instances, the TCR alpha extracellular domain, or fragment thereof, and the TCR beta extracellular domain, or fragment thereof, are connected by a disulfide bond. In some instances, the TCR alpha extracellular domain, or fragment thereof, comprises an alpha chain TRAC constant domain sequence and the TCR beta extracellular domain, or fragment thereof, comprises a beta chain TRBC1 or TRBC2 constant domain sequence. In some instances, Cys4 of the alpha chain TRAC constant domain sequence is modified by truncation or substitution and Cys2 of exon 2 of the beta chain TRBC1 or TRBC2 constant domain sequence is modified by truncation or substitution, thereby deleting a native disulfide bond. In some instances, Thr48 of the alpha chain TRAC constant domain sequence is mutated to Cys and Ser57 of the beta chain TRBC1 or TRBC2 constant domain sequence is mutated to Cys. In some instances, the modified TCR comprises a modified amino acid, a non-natural amino acid, a modified non-natural amino acid, or a combination thereof. In some instances, the modified amino acid or modified non-natural amino acid comprises a post-translational modification. In some instances, the target antigen is MAGE-A3 or titin. In some instances, the polypeptide of formula III binds to a target cell when L₃ is cleaved by the tumor specific protease. In some instances, P₃ inhibits binding of the modified TCR to the target cell when outside the tumor microenvironment. In some instances, the modified TCR has an increased binding affinity for its pMHC as compared to the binding affinity for the pMHC of an unmodified form of the TCR that does not have P₃ or L₃. In some instances, the modified TCR has an increased binding affinity for its pMHC that is at least 10× higher than the binding affinity for the pMHC of an unmodified form of the TCR that does not have P₃ or L₃. In some instances, the modified TCR has an increased binding affinity for its pMHC that is at least 100× higher than the binding affinity for the pMHC of an unmodified form of the TCR that does not have P₃ or L₃. In some instances, the modified TCR has an increased binding affinity for its pMHC as compared to the binding affinity for the pMHC of the modified TCR in which L₃ has been cleaved by the tumor specific protease. In some instances, the modified TCR has an increased binding affinity for its pMHC that is at least 10× higher than the binding affinity for the pMHC of the modified TCR in which L₃ has been cleaved by the tumor specific protease. In some instances, the modified TCR has an increased binding affinity for its pMHC that is at least 100× higher than the binding affinity for the pMHC of the modified TCR in which L₃ has been cleaved by the tumor specific protease.

Disclosed herein, in certain embodiments, are pharmaceutical compositions, comprising: (a) the modified TCR according to any of the disclosures herein; and (b) a pharmaceutically acceptable excipient.

Disclosed herein, in certain embodiments, are isolated recombinant nucleic acid molecules encoding a polypeptide comprising a formula III: T₃-L₃-P₃ (formula III) wherein: T₃ comprises either a TCR alpha extracellular domain, or fragment thereof, or a TCR beta extracellular domain, or fragment thereof, wherein T₃ binds to a target antigen and the TCR alpha extracellular domain or fragment thereof and the TCR beta extracellular domain, or fragment thereof contain an antigen binding site, P₃ is a peptide that reduces binding of T₃ to the target antigen when the modified TCR is outside of a tumor microenvironment and that does not reduce binding of T₃ to the target antigen when the modified TCR is inside the tumor microenvironment, and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an exemplary schematic of a T cell receptor (TCR) that does not comprise a peptide modification. Such TCRs bind to unique antigens that exist in abundance in tumor tissue. But, the unique antigens are also found in some healthy tissues, which can trigger systemic immune activation in a subject, and cause toxicity.

FIG. 2 is an exemplary ribbon diagram of an alpha polypeptide chain and a beta polypeptide chain of a TCR. The N-termini are highlighted as exemplary points of attachment for inserting the peptides described herein.

FIG. 3A-FIG. 3C shows exemplary schematics of modified TCRs in the soluble format in which the modified TCR is further conjugated to an effector domain. In these examples, the effector domain is an anti-CD3 moiety. FIGS. 3A-3C are exemplary schematics of modified TCRs with an effector domain. FIG. 3A depicts the modified TCR heterodimer conjugated to an anti-CD3 single-chain variable fragment (scFv) effector. FIG. 3B illustrates a format in which the modified TCR heterodimer is bound to an Fc that is also bound to an anti-CD3 single-chain variable fragment (scFv) effector. FIG. 3C illustrates a single polypeptide TCR format comprising a variable region of a TCR alpha extracellular domain and a variable region of the TCR beta extracellular domain wherein the single polypeptide is bound to an Fc that is also bound to an anti-CD3 single-chain variable fragment (scFv) effector.

FIG. 4 is an exemplary BLI sensorgram and affinity of TCR-1 binding to MAGE-A3 pMHC in realtime.

FIG. 5A-FIG. 50 are exemplary kinetic binding sensorgrams for TCR-1 binding to synthetic peptides.

FIG. 6 exemplifies binding of TCR-1 to peptides by ELISA.

FIG. 7A-FIG. 7M exemplify peptide inhibition of TCR-1 kinetic binding to MAGE-A3 pMHC.

FIG. 8 exemplifies dose dependent inhibition of TCR-1 binding to MAGE-A3 pMHC using peptides measured using BLI Octet instrument.

FIG. 9 exemplifies dose dependent inhibition of TCR-1 binding to MAGE-A3 pMHC using peptides measured in competitive binding ELISA.

FIG. 10 is an exemplary BLI sensorgram and affinity of TCR-1 binding to Peptide-5 in realtime.

FIG. 11 exemplifies TCR-1 binding of MAGE-A3 pMHC or Peptide-5 by ELISA.

FIG. 12A-FIG. 12H are exemplary sensorgrams for Peptide-5 dose dependent inhibition of kinetic binding of TCR-1 to cognate MAGE-A3 pMHC.

FIG. 13 is an exemplary IC50 curve for Peptide-5 dose dependent inhibition of kinetic binding of TCR-1 to cognate MAGE-A3 pMHC.

FIG. 14 exemplifies Peptide-5 dose dependent inhibition of TCR-1 binding to its cognate MAGE-A3 pMHC by competitive ELISA.

FIG. 15A-FIG. 15D are exemplary BLI sensorgrams of TCR-1, TCR-8, TCR-9, and TCR-10 TCR binding to Peptide-5 in realtime.

FIG. 16A-FIG. 16E are exemplary BLI sensorgrams of TCR-1, TCR-8, TCR-9, and TCR-10 TCRs at 100 uM binding to saturating levels Peptide-5 loaded on streptavidin biosensors in real time.

FIG. 17A-FIG. 17D exemplify BLI sensorgrams of TCR-1 binding to Peptide-5 at acidic pH in realtime.

FIG. 18 exemplifies TCR-1 binding to Peptide-5 at acidic pH by ELISA.

FIG. 19A-FIG. 19G exemplify Peptide-5 alanine scan peptides evaluation in kinetic binding experiments against TCR-1.

FIG. 20 exemplifies Peptide-5 alanine scan peptides evaluation for binding to TCR-1 by ELISA.

FIG. 21A-FIG. 21I exemplify Peptide-5 alanine scan peptides evaluation for dose dependent inhibition of TCR-1 binding to MAGE-A3 pMHC by kinetic measurements.

FIG. 22 exemplifies Peptide-5 alanine scan peptides evaluation for dose dependent inhibition of TCR-1 binding to MAGE-A3 pMHC by ELISA.

FIG. 23A-FIG. 23C exemplify BLI sensorgrams pre and post urokinase treatment of TCR-1, TCR-4, and TCR-5 binding to MAGE-A3 pMHC in realtime.

FIG. 24A-FIG. 24C exemplify BLI sensorgrams of TCR-1, TCR-2, and TCR-3 binding to MAGE-A3 pMHC in realtime.

FIG. 25A-FIG. 25C exemplify BLI sensorgrams of TCR-1, TCR-4 and TCR-5 binding to Titin pMHC in realtime.

FIG. 26 exemplifies binding of Peptide-5 masked TCRs with a cleavable linker, TCR-4 and TCR-5, relative to unmasked TCR, TCR-1, to MAGE-A3 pMHC.

FIG. 27 exemplifies binding of Peptide-5 masked TCRs with a cleavable linker, TCR-4 and TCR-5, relative to unmasked TCR, TCR-1, to Titin pMHC.

FIG. 28A-FIG. 28C exemplifies BLI sensorgrams pre and post urokinase treatment of TCR-1, TCR-6 and TCR-7 binding to MAGE-A3 pMHC in realtime.

FIG. 29A-FIG. 29B exemplifies BLI sensorgrams of TCR-1, TCR-4, or TCR-5 binding to cognate MAGE-A3 pMHC pre and post 24 hour incubation in human serum.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

“Transmembrane domain”, as used herein, refers to the region of a receptor which crosses the plasma membrane. Examples include the transmembrane region of a transmembrane protein (for example a Type 1 transmembrane protein), an artificial hydrophobic sequence, and a combination thereof.

“Fragment” as used herein refers to a peptide or a polypeptide that comprises less than the full length amino acid sequence.

“Antigen-binding site” as used herein refers to the region of a polypeptide that interacts with an antigen. The antigen binding site includes amino acid residues that interact directly with an antigen and those amino acid residues that are within proximity to the antigen but that may not interact directly with the antigen.

“Target antigen” as used herein refers to a molecule that binds to a variable region of the TCR alpha extracellular domain or the variable region of the TCR beta extracellular domain or both.

### T Cell Receptor (TCR)

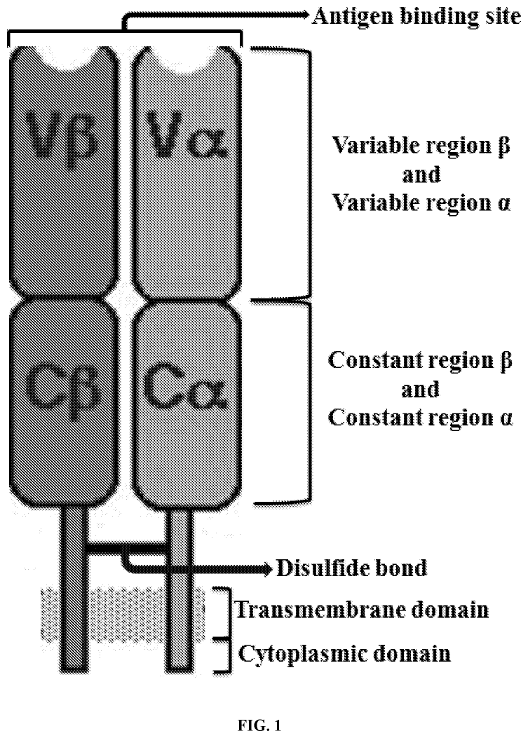

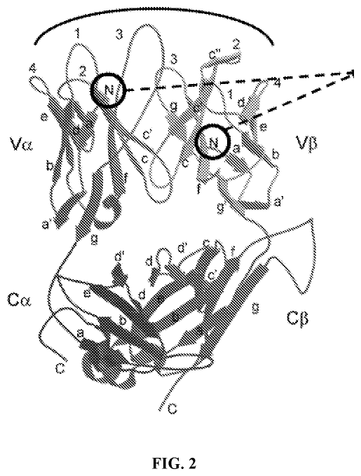

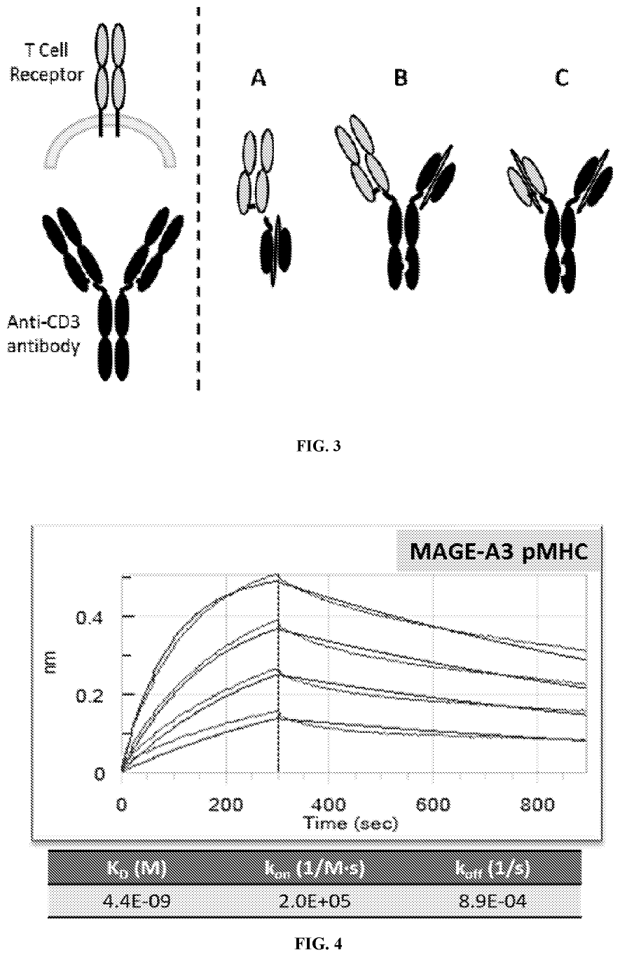

Native TCRs are transmembrane receptors expressed on the surface of T cells that recognize antigens bound to major histocompatibility complex molecules (MHC). Native TCRs are heterodimeric and comprise an alpha polypeptide chain and a beta polypeptide chain linked through a disulfide bond (FIG. 1). The alpha polypeptide chain and the beta polypeptide chain are expressed as part of a complex with accessory proteins which include, for example, two CD3 epsilon polypeptides, one CD3 gamma polypeptide, one CD3 delta polypeptide, and two CD3 zeta polypeptides. When a TCR engages with a target antigen and MHC, the T cell is activated resulting in a series of signaling events mediated by associated enzymes, co-receptors, adapter molecules, and activated or released transcription factors.

In native TCRs, the alpha polypeptide chain and the beta polypeptide chain comprise an extracellular domain, a transmembrane domain, and a cytoplasmic domain. Each extracellular domain comprises a variable region (V), a joining region (J), and a constant region (C). The constant region is N-terminal to the transmembrane domain, and the transmembrane domain is N-terminal to the cytoplasmic domain. The variable regions of both the alpha polypeptide chain and the beta polypeptide chain comprise three hypervariable or complementarity determining regions (CDRs). The beta polypeptide chain usually contains a short diversity region between the variable and joining regions. The three CDRs are embedded into a framework sequence, with one CDR being the hypervariable region named CDR3. The alpha chain variable region (Va) and the beta chain variable region (VP) are of several types that are distinguished by their framework sequences, CDR1 and CDR2 sequences, and a partly defined CDR3 sequence.

TCRs are described using the International Immunogenetics (IMGT) TCR nomenclature. The Va in IMGT nomenclature is referred to by a unique “TRAV” number. In the same way, Vβ is referred to by a unique “TRBV” number. The corresponding joining and constant regions are referred to as TRAJ and TRAC, respectively for the α joining and constant regions, and TRBJ and TRBC, respectively for the β joining and constant regions. The sequences defined by the IMGT nomenclature are known in the art and are contained within the online IMGT public database.

### Polypeptides of Modified T Cell Receptors (TCRs)

In some embodiments, as described herein, are modified TCRs. In some embodiments, a TCR is modified such that the alpha polypeptide chain or the beta polypeptide chain, or both the alpha polypeptide chain and the beta polypeptide chain comprise a peptide that conceals, sterically blocks, or inhibits the antigen binding site of the alpha polypeptide chain or the beta polypeptide chain from engaging with its target antigen. In some embodiments, the peptide conceals, sterically blocks, or inhibits the antigen binding site of the alpha polypeptide chain or the beta polypeptide chain from engaging with the target antigen when the modified TCR is outside a tumor microenvironment. In some embodiments, when the modified TCR is inside a tumor microenvironment, the peptide is cleaved by a protease that is specific to the tumor microenvironment, thereby exposing the antigen binding site of the alpha polypeptide chain or beta polypeptide chain. Without being bound by a particular theory, the selective cleavage of the peptide within tumor microenvironments creates specificity for the modified TCR to engage with the target antigen in the tumor microenvironment, while minimizing engagement with the target antigen outside the tumor microenvironment thus creating an improved safety profile of the modified TCR.

In some embodiments, the peptide, a linking moiety, and the alpha polypeptide chain or the beta polypeptide chains are expressed as a single transcript. In some embodiments, the linking moiety is cleavable by a protease that is specific to the tumor microenvironment. In some embodiments, the linking moiety is C-terminal to the peptide, and the linking moiety is bound to the N-terminus of the alpha polypeptide chain or the beta polypeptide chain, thereby connecting the peptide and the alpha polypeptide chain or beta polypeptide chain. In some embodiments, the linking moiety, which is connected to the peptide, is bound to the alpha polypeptide chain or beta polypeptide chain at a location other than the N-terminus of the alpha polypeptide chain or beta polypeptide chain. In some embodiments, the linking moiety is coupled to the alpha polypeptide chain or beta polypeptide chain through a cysteine-cysteine disulfide bridge. In some embodiments, the linking moiety is bound to the alpha polypeptide chain or beta polypeptide chain through site specific modification. Methods for site specific modification of proteins include, but are not limited to, cysteine conjugation, glycoconjugation, unnatural or noncanonical amino acid incorporation, transglutaminase tags, sortase tags, and aldehyde tags.

In some embodiments, as described herein, the modified TCR comprises a polypeptide comprising a TCR alpha extracellular domain, or a fragment thereof, and a transmembrane domain, and a second polypeptide comprising a TCR beta extracellular domain, or fragment thereof, and a transmembrane domain. In some embodiments, the TCR alpha extracellular domain, or fragment thereof, or the TCR beta extracellular domain, or fragment thereof, or both comprise a peptide which conceals, sterically blocks, or inhibits the antigen binding site from engaging with the target antigen outside of a tumor microenvironment. In some embodiments, the peptide is cleaved by a tumor specific protease when the modified TCR is inside a tumor microenvironment.

In some embodiments, the TCR alpha extracellular domain, or fragment thereof comprises a variable region. In some embodiments, the TCR alpha extracellular domain, or fragment thereof comprises a variable region, a joining region, and a constant region. In some embodiments, the TCR alpha extracellular domain is a full length TCR alpha extracellular domain.

In some embodiments, the TCR beta extracellular domain, or fragment thereof comprises a variable region. In some embodiments, the TCR beta extracellular domain, or fragment thereof comprises a variable region, a joining region, and a constant region. In some embodiments, the TCR beta extracellular domain is a full length TCR beta extracellular domain.

In some embodiments, the modified TCR contains a hinge region linking the TCR extracellular domain with the transmembrane domain.

In some embodiments, the transmembrane domain provides for insertion of the modified TCR to be expressed on the surface of a cell. Non-limiting examples of transmembrane sequences include, but are not limited to: a) CD8 beta derived: GLLVAGVLVLLVSLGVAIHLCC (SEQ ID NO: 40); b) CD4 derived: ALIVLGGVAGLLLFIGLGIFFCVRC (SEQ ID NO: 41); c) CD3 zeta derived: LCYLLDGILFIYGVILTALFLRV (SEQ ID NO: 42); d) CD28 derived: WVLVVVGGVLACYSLLVTVAFIIFWV (SEQ ID NO: 43); e) CD134 (OX40) derived: AAILGLGLVLGLLGPLAILLALYLL (SEQ ID NO: 44); f) CD7 derived: ALPAALAVISFLLGLGLGVACVLA (SEQ ID NO: 45); g) native TCR alpha polypeptide chain transmembrane sequences; h) native TCR beta polypeptide chain transmembrane sequences, or a combination thereof.

In some embodiments, the modified TCRs described herein further comprise modifications in the TCR alpha extracellular domain or the TCR beta extracellular domain, wherein the modifications inhibit mispairing of the modified TCRs with the endogenous TCRs. In some embodiments, the modified TCRs described herein further comprise modifications in the TCR alpha extracellular domain and the TCR beta extracellular domain, wherein the modifications inhibit mispairing of the modified TCRs with the endogenous TCRs. In some embodiments, the modifications are in the TCR alpha constant domain or in the TCR beta constant domain. In some embodiments, the modifications are in the TCR alpha constant domain and in the TCR beta constant domain. In some embodiments, the modifications comprise interchanging the TCR alpha constant domain and the TCR beta constant domain. In some embodiments, the modifications comprise replacing the TCR alpha constant domain and the TCR beta constant domain with the corresponding domains from TCR gamma and delta.

In some embodiments, the polypeptide comprising the TCR alpha extracellular domain, or fragment thereof, further comprises a cytoplasmic domain C-terminal to the transmembrane domain. In some embodiments, the second polypeptide comprising the TCR beta extracellular domain, or fragment thereof, further comprises a cytoplasmic domain C-terminal to the transmembrane domain.

In some embodiments, the cytoplasmic domain comprises at least one costimulatory domain. In some embodiments, the costimulatory domain is 4-1BB or CD28. In some embodiments, the cytoplasmic domain comprises two costimulatory domains. In some embodiments, the cytoplasmic domain comprises more than two costimulatory domains. In some embodiments, the costimulatory domain, includes, but is not limited to C27, CD28, ICOS, 4-1BB, OX40 or CD3ζ. In some embodiments, the cytoplasmic domain includes ZAP70. In some embodiments, the cytoplasmic domain includes LAT. In some embodiments, the cytoplasmic domain comprises CD3ζ, ZAP70, and LAT.

In some embodiments, the modified TCR is a soluble TCR. In some embodiments, the modified TCR comprises a polypeptide comprising a TCR alpha extracellular domain, or a fragment thereof, and a second polypeptide comprising a TCR beta extracellular domain, or fragment thereof, wherein either the TCR alpha extracellular domain or the TCR beta extracellular domain or both comprise a peptide which conceals, sterically blocks, or inhibits the antigen binding site from engaging with the target antigen outside of a tumor microenvironment. In some embodiments, the polypeptide comprising the TCR alpha extracellular domain, or fragment thereof, further comprises a truncated transmembrane domain. In some embodiments, the polypeptide comprising the TCR alpha extracellular domain, or fragment thereof lacks a transmembrane domain. In some embodiments, the second polypeptide comprising the TCR beta extracellular domain, or fragment thereof, further comprises a truncated transmembrane domain. In some embodiments, the second polypeptide comprising the TCR beta extracellular domain, or fragment thereof, lacks a transmembrane domain. In some embodiments, the TCR alpha extracellular domain, or fragment thereof and TCR beta extracellular domain, or fragment thereof, are mutated to delete the native cysteines which form the native disulfide linkage of the heterodimer. In some embodiments, the polypeptide comprising the TCR alpha extracellular domain, or fragment thereof, further comprises an anti-CD3 single-chain variable fragment effector. In some embodiments, the second polypeptide comprising the TCR beta extracellular domain, or fragment thereof, further comprises an anti-CD3 single-chain variable fragment effector.

In some embodiments, the modified TCR is a heterodimer of an alpha polypeptide chain and a beta polypeptide chain (α/β heterodimer). In some embodiments, the TCR comprises a single polypeptide comprising a variable region of a TCR alpha extracellular domain (Vα), or a fragment thereof, and a variable region of a TCR beta extracellular domain (Vβ), or a fragment thereof, instead of an α/β heterodimer. In some embodiments, the single polypeptide further comprises a sequence that connects Vα and Vβ. In some embodiments, the single polypeptide comprises a constant region of the TCR alpha extracellular domain (Ca) or a constant region of the TCR beta extracellular domain (Cβ) or a combination thereof.

### Modified TCRs Expressed on the Surface of the Cell

Disclosed herein, in certain embodiments, are modified T cell receptors (TCR) comprising a polypeptide of formula I:

T₁-L₁-P₁ (formula I)

wherein T₁ comprises a transmembrane domain and either a TCR alpha extracellular domain, or fragment thereof, or a TCR beta extracellular domain, or fragment thereof, wherein T₁ binds to a target antigen and the TCR alpha extracellular domain or fragment thereof and the TCR beta extracellular domain, or fragment thereof contain an antigen binding site, P₁ is a peptide that reduces binding of T₁ to the target antigen when the modified TCR is outside of a tumor microenvironment and that does not reduce binding of T₁ to the target antigen when the modified TCR is inside the tumor microenvironment, and L₁ is a linking moiety that connects T₁ to P₁ and L₁ is bound to T₁ at the N-terminus of T₁, wherein the modified TCR is a functional TCR when inside the tumor microenvironment and is a nonfunctional TCR when outside the tumor microenvironment and P₁ or L₁ is a substrate for a tumor specific protease. In some embodiments, T₁ comprises the TCR alpha extracellular domain, or fragment thereof, and the modified TCR further comprises a second polypeptide comprising a transmembrane domain and a TCR beta extracellular domain, or fragment thereof wherein the TCR beta extracellular domain or fragment thereof contains an antigen binding site. In some embodiments, T₁ comprises the TCR beta extracellular domain, or fragment thereof, and the modified TCR further comprises a second polypeptide comprising a transmembrane domain and a TCR alpha extracellular domain, or fragment thereof wherein the TCR alpha extracellular domain or fragment thereof contains an antigen binding site.

In some embodiments, T₁ comprises the TCR alpha extracellular domain, or fragment thereof, and the modified TCR further comprises a second polypeptide of formula II:

T₂-L₂-P₂ (formula II)

wherein T₂ comprises a transmembrane domain and a TCR beta extracellular domain, or fragment thereof, wherein T₂ binds to the target antigen, and the TCR beta extracellular domain or fragment thereof contains an antigen binding site, P₂ is a peptide that reduces binding of T₂ to the target antigen when the modified TCR is outside of a tumor microenvironment and that does not reduce binding of T₂ to the target antigen when the modified TCR is inside the tumor microenvironment, and L₂ is a linking moiety that connects T₂ to P₂ and L₂ is bound to T₂ at the N-terminus of T₂, wherein P₂ or L₂ is a substrate for a tumor specific protease.

In some embodiments, the target antigen includes, but is not limited to MAGE-A3, NY-ESO-1, gp100, WT1, and tyrosinase. In some embodiments, the target antigen is MAGE-A3. In some embodiments, the target antigen is NY-ESO-1. In some embodiments, the target antigen is gp100. In some embodiments, the target antigen is WT1. In some embodiments, the target antigen is tyrosinase.

Peptide (P₁ and P₂)

In some embodiments, P₁ and P₂ bind to T₁ and T₂ thereby concealing the antigen binding sites of T₁ and T₂ from engaging with the target antigen. In some embodiments, P₁ binds to T₁. In some embodiments, P₁ binds to T₁ and T₂. In some embodiments, P₁ binds to T₂. In some embodiments, P₂ binds to T₂. In some embodiments, P₂ binds to T₁ and T₂. In some embodiments, P₂ binds to T₁. In some embodiments, P₁ and P₂ bind to T₁ and T₂ when the modified TCR is outside of a tumor microenvironment. In some embodiments, when the modified TCR is inside the tumor microenvironment, P₁ and P₂ are cleaved from their respective polypeptide chains, thereby exposing the antigen binding sites of T₁ and T₂.

In some embodiments, P₁ is bound to T₁ through ionic interactions, electrostatic interactions, hydrophobic interactions, Pi-stacking interactions, and H-bonding interactions, or a combination thereof when the modified TCR is outside the tumor microenvironment. In some embodiments, P₂ is bound to T₂ through ionic interactions, electrostatic interactions, hydrophobic interactions, Pi-stacking interactions, and H-bonding interactions, or a combination thereof when the modified TCR is outside the tumor microenvironment. In some embodiments, P₁ is bound to T₁ at or near the antigen binding site when the modified TCR is outside the tumor microenvironment. In some embodiments, P₂ is bound to T₂ at or near the antigen binding site when the modified TCR is outside the tumor microenvironment. In some embodiments, P₁ inhibits the binding of T₁ to the target antigen when the modified TCR is outside the tumor microenvironment, and P₁ does not inhibit the binding of T₁ to the target antigen when the modified TCR is inside the tumor microenvironment. In some embodiments, P₂ inhibits the binding of T₂ to the target antigen when the modified TCR is outside the tumor microenvironment, and P₂ does not inhibit the binding of T₂ to the target antigen when the modified TCR is inside the tumor microenvironment. In some embodiments, P₁ sterically blocks T₁ from binding to the target antigen when the modified TCR is outside the tumor microenvironment. In some embodiments, P₂ sterically blocks T₂ from binding to the target antigen when the modified TCR is outside the tumor microenvironment. In some embodiments, P₁ is removed from the antigen binding site, and the antigen binding site of T₁ is exposed when the modified TCR is inside the tumor microenvironment. In some embodiments, P₂ is removed from the antigen binding site, and the antigen binding site of T₁ is exposed when the modified TCR is inside the tumor microenvironment.

In some embodiments, P₁ is a peptide sequence at least 5 amino acids in length. In some embodiments, P₁ is a peptide sequence at least 6 amino acids in length. In some embodiments, P₁ is a peptide sequence at least 10 amino acids in length. In some embodiments, P₁ is a peptide sequence at least 20 amino acids in length. In some embodiments, P₁ is a linear peptide. In some embodiments, P₁ is a cyclic peptide. In some embodiments, P₁ is resistant to cleavage by a protease while L₁ is cleavable by a tumor specific protease.

In some embodiments, P₁ is not a natural binding partner of T₁ or T₂. In some instances, P₁ is a modified binding partner of T₁ and T₂ and contains amino acid changes that at least slightly decrease affinity and/or avidity of binding to T₁ and T₂. In some embodiments, P₁ contains no or substantially no homology to T₁ and T₂ natural binding partner. In some embodiments, P₁ contains at least 5%, 10%, 15%, 20%, 25%, 30%, 35%, 40%, 45%, 50%, 55%, 60%, 65%, 70%, 75%, or 80% sequence identity to the natural binding partner of T₁ and T₂. In some embodiments, P₁ contains at least 5%, 10%, 15%, 20%, 25%, 30%, 35%, 40%, 45%, 50%, 55%, 60%, 65%, 70%, 75%, or 80% sequence identity to the natural binding partner of T₁ and T₂. In some embodiments, P₁ contains at least 5%, 10%, 15%, 20%, 25%, 30%, 35%, 40%, 45%, 50%, 55%, 60%, 65%, 70%, 75%, or 80% sequence identity to the target antigen.

In some embodiments, P₂ is a peptide sequence at least 5 amino acids in length. In some embodiments, P₂ is a peptide sequence at least 6 amino acids in length. In some embodiments, P₂ is a peptide sequence at least 10 amino acids in length. In some embodiments, P₂ is a peptide sequence at least 20 amino acids in length. In some embodiments, P₂ is a linear peptide. In some embodiments, P₂ is a cyclic peptide. In some embodiments, P₂ is resistant to cleavage by a protease while L₂ is cleavable by a tumor specific protease.

In some embodiments, P₂ is not a natural binding partner of T₁ or T₂. In some instances, P₂ is a modified binding partner of T₁ and T₂ and contains amino acid changes that at least slightly decrease affinity and/or avidity of binding to T₁ and T₂. In some embodiments, P₂ contains no or substantially no homology to T₁ and T₂ natural binding partner. In some embodiments, P₂ contains at least 5%, 10%, 15%, 20%, 25%, 30%, 35%, 40%, 45%, 50%, 55%, 60%, 65%, 70%, 75%, or 80% sequence identity to the natural binding partner of T₁ and T₂. In some embodiments, P₂ contains at least 5%, 10%, 15%, 20%, 25%, 30%, 35%, 40%, 45%, 50%, 55%, 60%, 65%, 70%, 75%, or 80% sequence identity to the target antigen.

In some embodiments, P₁ or P₂ or P₁ and P₂ are substrates for a tumor specific protease. In some embodiments, the tumor specific protease is a metalloprotease, serine protease, cysteine protease, threonine protease, and aspartic protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, P₁ or P₂ or P₁ and P₂ comprise a modified amino acid or non-natural amino acid, or a modified non-natural amino acid, or a combination thereof. In some embodiments, the modified amino acid or a modified non-natural amino acid comprises a post-translational modification. In some embodiments P₁ or P₂ or P₁ and P₂ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁ or P₂ or P₁ and P₂ including the peptide backbone, the amino acid side chains, and the terminus.

Linking Moiety (L₁ and L₂)

In some embodiments, L₁ is cleavable by a protease. In some embodiments, L₁ is cleavable by a protease that is specific to a particular microenvironment. In some embodiments, L₁ is resistant to protease cleavage, while P₁ is cleavable by a protease. In some embodiments, the protease is metalloprotease, serine protease, cysteine protease, threonine protease, and aspartic protease. In some embodiments, L₁ is cleavable by a tumor specific protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, L₂ is cleavable by a protease. In some embodiments, L₂ is cleavable by a protease that is specific to a particular microenvironment. In some embodiments, L₂ is resistant to protease cleavage, while P₂ is cleavable by a protease. In some embodiments, the protease is a metalloprotease, serine protease, cysteine protease, threonine protease, and aspartic protease. In some embodiments, L₂ is cleavable by a tumor specific protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, L₁ is a peptide sequence having at least 5 to no more than 50 amino acids. In some embodiments, L₁ has a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₁ has a formula comprising (GGSGGD)ₙ, wherein n is an integer from 2 to 6 (SEQ ID NO: 8). In some embodiments, L₁ has a formula comprising (GGSGGE)ₙ, wherein n is an integer from 2 to 6 (SEQ ID NO: 9). In some embodiments, L₁ has a formula comprising (GGGSGSGGGGS)ₙ, wherein n is an integer from 1 to 3 (SEQ ID NO: 6). In some embodiments, L₁ has a formula comprising (GGGGGPGGGGP)ₙ, wherein n is an integer from 1 to 3 (SEQ ID NO: 7). In some embodiments, L₁ has a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₁ comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₁ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₁ comprises an MMP cleavable amino acid sequence. In some embodiments, the MMP cleavable amino acid sequence is PLGLWA (SEQ ID NO: 19). In some embodiments, L₁ comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₁ comprises the sequence L₁ₓ-L_{1c}-L_{1z} wherein L₁ is a cleavable sequence. In some embodiments, L_{1c} comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L_{1c} comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L_{1c} comprises an MMP cleavable amino acid sequence. In some embodiments, the MMP cleavable amino acid sequence is PLGLWA (SEQ ID NO: 19). In some embodiments, L_{1c} comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₁ₓ or L_{1z} have a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₁ₓ or L_{1z} have a formula comprising (GGSGGD)ₙ, wherein n is an integer from 2 to 6 (SEQ ID NO: 8). In some embodiments, L₁ₓ or L_{1z} have a formula comprising (GGSGGE)ₙ, wherein n is an integer from 2 to 6 (SEQ ID NO: 9). In some embodiments, L₁ₓ or L_{1z} have a formula comprising (GGGSGSGGGGS)ₙ, wherein n is an integer from 1 to 3 (SEQ ID NO: 6). In some embodiments, L₁ₓ or L_{1z} have a formula comprising (GGGGGPGGGGP)ₙ, wherein n is an integer from 1 to 3 (SEQ ID NO: 7). In some embodiments, L₁ₓ or L_{1z} have a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₂ is a peptide sequence having at least 5 to no more than 50 amino acids. In some embodiments, L₂ has a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₂ has a formula comprising (GGSGGD)ₙ, wherein n is an integer from 2 to 6 (SEQ ID NO: 8). In some embodiments, L₂ has a formula comprising (GGSGGE)ₙ, wherein n is an integer from 2 to 6 (SEQ ID NO: 9). In some embodiments, L₂ has a formula comprising (GGGSGSGGGGS)ₙ, wherein n is an integer from 1 to 3 (SEQ ID NO: 6). In some embodiments, L₂ has a formula comprising (GGGGGPGGGGP)ₙ, wherein n is an integer from 1 to 3 (SEQ ID NO: 7). In some embodiments, L₂ has a formula selected from (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); (G_{z}X)ₙ, wherein X is serine, aspartic acid, glutamic acid, threonine, or proline and n is at least 15, and z is between 1 and 20 (SEQ ID NO: 28).

In some embodiments, L₂ comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₂ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₂ comprises an MMP cleavable amino acid sequence. In some embodiments, the MMP cleavable amino acid sequence is PLGLWA (SEQ ID NO: 19). In some embodiments, L₂ comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₂ comprises the sequence L₂ₓ-L_{2c}-L_{2z} wherein L_{2c} is a cleavable sequence. In some embodiments, L_{2c} comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L_{2c} comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₂ comprises an MMP cleavable amino acid sequence. In some embodiments, the MMP cleavable amino acid sequence is PLGLWA (SEQ ID NO: 19). In some embodiments, L_{2c} comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₂ₓ or L_{2z} have a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₂ₓ or L_{2z} have a formula comprising (GGSGGD)ₙ, wherein n is an integer from 2 to 6 (SEQ ID NO: 8). In some embodiments, L₂ₓ or L_{2z} have a formula comprising (GGSGGE)ₙ, wherein n is an integer from 2 to 6 (SEQ ID NO: 9). In some embodiments, L₂ₓ or L_{2z} have a formula comprising (GGGSGSGGGGS)ₙ, wherein n is an integer from 1 to 3 (SEQ ID NO: 6). In some embodiments, L₂ₓ or L_{2z} have a formula comprising (GGGGGPGGGGP)ₙ, wherein n is an integer from 1 to 3 (SEQ ID NO: 7). In some embodiments, L₂ₓ or L_{2z} have a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₁ or L₂ or L₁ and L₂ comprise a modified amino acid or non-natural amino acid, or a modified non-natural amino acid, or a combination thereof. In some embodiments, the modified amino acid or a modified non-natural amino acid comprises a post-translational modification. In some embodiments, L₁ or L₂ or L₁ and L₂ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₁ or L₂ or L₁ and L₂ including the peptide backbone, or the amino acid side chains.

TCR Alpha Extracellular Domain and a TCR Beta Extracellular Domain and Transmembrane Domain (T₁ and T₂)

In some embodiments, the TCR alpha extracellular domain, or fragment thereof comprises a variable region. In some embodiments, the TCR alpha extracellular domain, or fragment thereof comprises a variable region, a joining region, and a constant region. In some embodiments, the TCR alpha extracellular domain, or fragment thereof, comprises three hyper-variable complementarity determining regions (CDRs) within the variable region. In some embodiments, at least one CDR comprises a mutation to increase binding affinity or binding specificity to the target antigen or to increase binding affinity and binding specificity to the target antigen. In some embodiments, there are 2-20, 3-15, 4-12, or 4-10 mutations in one or two CDRs.

In some embodiments, the TCR alpha extracellular domain, or fragment thereof, comprises a modified amino acid. In some embodiments, the modified amino acid comprises a post-translational modification. In some embodiments, the TCR alpha extracellular domain, or fragment thereof, comprises a non-natural amino acid or a modified non-natural amino acid, or combination thereof. In some embodiments, the modified non-natural amino acid comprises a post-translational modification.

In some embodiments, the TCR beta extracellular domain, or fragment thereof comprises a variable region. In some embodiments, the TCR beta extracellular domain, or fragment thereof comprises a variable region, a joining region, and a constant region. In some embodiments, the TCR beta extracellular domain or fragment thereof, comprises three hyper-variable complementarity determining regions (CDRs). In some embodiments, at least one CDR comprises a mutation to increase binding affinity or binding specificity to the target antigen or to increase binding affinity and binding specificity to the target antigen. In some embodiments, there are 2-20, 3-15, 4-12, or 4-10 mutations in one or two CDRs.

In some embodiments, the TCR beta extracellular domain or fragment thereof, comprises a modified amino acid. In some embodiments, the modified amino acid comprises a post-translational modification. In some embodiments, the TCR beta extracellular domain, or fragment thereof, comprises a non-natural amino acid or a modified non-natural amino acid, or combination thereof. In some embodiments, the modified non-natural amino acid comprises a post-translational modification.

In some embodiments, T₁ comprises a full length TCR alpha polypeptide chain. In some embodiments, T₁ comprises a full length TCR beta polypeptide chain. In some embodiments, T₂ comprises a full length TCR beta chain polypeptide. In some embodiments, T₁ comprises a full length TCR alpha polypeptide chain, and the modified TCR further comprises a second polypeptide comprising a full length TCR beta polypeptide chain.

### Soluble Modified TCRs

Disclosed herein, in certain embodiments, are modified T cell receptors (TCR) comprising a polypeptide of formula III:

T₃-L₃-P₃ (formula III)

wherein T₃ comprises either a TCR alpha extracellular domain, or fragment thereof, or a TCR beta extracellular domain, or fragment thereof, wherein T₃ binds to a target antigen, and the TCR alpha extracellular domain or fragment thereof and the TCR beta extracellular domain, or fragment thereof contain an antigen binding site; P₃ is a peptide that reduces binding of T₃ to the target antigen when the modified TCR is outside of a tumor microenvironment and that does not reduce binding of T₃ to the target antigen when the modified TCR is inside the tumor microenvironment, and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease. In some embodiments, T₃ comprises the TCR alpha extracellular domain, or fragment thereof, and the modified TCR further comprises a second polypeptide comprising a TCR beta extracellular domain, or fragment thereof wherein the TCR beta extracellular domain or fragment thereof contains an antigen binding site. In some embodiments, T₃ comprises the TCR beta extracellular domain, or fragment thereof, and the modified TCR further comprises a second polypeptide comprising a TCR alpha extracellular domain, or fragment thereof wherein the TCR alpha extracellular domain or fragment thereof contains an antigen binding site.

In some embodiments, T₃ comprises the TCR alpha extracellular domain, or fragment thereof, and the modified TCR further comprises a second polypeptide of formula IV:

T₄-L₄-P₄ (formula IV)

wherein T₄ comprises a TCR beta extracellular domain, or fragment thereof, wherein T₄ binds to the target antigen, and the TCR beta extracellular domain or fragment thereof contains an antigen binding site; P₄ is a peptide that reduces binding of T₄ to the target antigen when the modified TCR is outside of a tumor microenvironment and that does not reduce binding of T₄ to the target antigen when the modified TCR is inside the tumor microenvironment, and L₄ is a linking moiety that connects T₄ to P₄ and L₄ is bound to T₄ at the N-terminus of T₄, wherein P₄ or L₄ is a substrate for a tumor specific protease.

In some embodiments, the target antigen includes, but is not limited to MAGE-A3, NY-ESO-1, gp100, WT1, and tyrosinase. In some embodiments, the target antigen is MAGE-A3. In some embodiments, the target antigen is NY-ESO-1. In some embodiments, the target antigen is gp100. In some embodiments, the target antigen is WT1. In some embodiments, the target antigen is tyrosinase.

Peptide (P₃ and P₄)

In some embodiments, P₃ and P₄ bind to T₃ and T₄ thereby concealing the antigen binding sites of T₃ and T₄ from engaging with the target antigen. In some embodiments, P₃ binds to T₃. In some embodiments, P₃ binds to T₃ and T₄. In some embodiments, P₃ binds to T₄. In some embodiments, P₄ binds to T₄. In some embodiments, P₄ binds to T₃ and T₄. In some embodiments, P₄ binds to T₃. In some embodiments, P₃ and P₄ bind to T₃ and T₄ when the modified TCR is outside of a tumor microenvironment. In some embodiments, when the modified TCR is inside the tumor microenvironment, P₃ and P₄ are cleaved from their respective polypeptide chains, thereby exposing the antigen binding sites of T₃ and T₄.

In some embodiments, P₃ is bound to T₄ through ionic interactions, electrostatic interactions, hydrophobic interactions, Pi-stacking interactions, and H-bonding interactions, or a combination thereof when the modified TCR is outside the tumor microenvironment. In some embodiments, P₄ is bound to T₄ through ionic interactions, electrostatic interactions, hydrophobic interactions, Pi-stacking interactions, and H-bonding interactions, or a combination thereof when the modified TCR is outside the tumor microenvironment. In some embodiments, P₃ is bound to T₃ at or near the antigen binding site when the modified TCR is outside the tumor microenvironment. In some embodiments, P₄ is bound to T₄ at or near the antigen binding site when the modified TCR is outside the tumor microenvironment. In some embodiments, P₃ inhibits the binding of T₃ to the target antigen when the modified TCR is outside the tumor microenvironment, and P₃ does not inhibit the binding of T₃ to the target antigen when the modified TCR is inside the tumor microenvironment. In some embodiments, P₄ inhibits the binding of T₄ to the target antigen when the modified TCR is outside the tumor microenvironment, and P₄ does not inhibit the binding of T₄ to the target antigen when the modified TCR is inside the tumor microenvironment. In some embodiments, P₃ sterically blocks T₃ from binding to the target antigen when the modified TCR is outside the tumor microenvironment. In some embodiments, P₄ sterically blocks T₄ from binding to the target antigen when the modified TCR is outside the tumor microenvironment. In some embodiments, P₃ is removed from the antigen binding site, and the antigen binding site of T₃ is exposed when the modified TCR is inside the tumor microenvironment. In some embodiments, P₄ is removed from the antigen binding site, and the antigen binding site of T₄ is exposed when the modified TCR is inside the tumor microenvironment.

In some embodiments, P₃ is a peptide sequence at least 5 amino acids in length. In some embodiments, P₃ is a peptide sequence at least 6 amino acids in length. In some embodiments, P₃ is a peptide sequence at least 10 amino acids in length. In some embodiments, P₃ is a peptide sequence at least 20 amino acids in length. In some embodiments, P₃ is a linear peptide. In some embodiments, P₃ is a cyclic peptide. In some embodiments, P₃ is resistant to cleavage by a protease while L₃ is cleavable by a tumor specific protease.

In some embodiments, P₃ is not a natural binding partner of T₃ or T₄. In some instances, P₃ is a modified binding partner of T₃ and T₄ and contains amino acid changes that at least slightly decrease affinity and/or avidity of binding to T₃ and T₄. In some embodiments, P₃ contains no or substantially no homology to T₃ and T₄ natural binding partner. In some embodiments, P₃ contains at least 5%, 10%, 15%, 20%, 25%, 30%, 35%, 40%, 45%, 50%, 55%, 60%, 65%, 70%, 75%, or 80% sequence identity to the natural binding partner of T₃ and T₄. In some embodiments, P₃ contains at least 5%, 10%, 15%, 20%, 25%, 30%, 35%, 40%, 45%, 50%, 55%, 60%, 65%, 70%, 75%, or 80% sequence identity to the natural binding partner of T₃ and T₄. In some embodiments, P₃ contains at least 5%, 10%, 15%, 20%, 25%, 30%, 35%, 40%, 45%, 50%, 55%, 60%, 65%, 70%, 75%, or 80% sequence identity to the target antigen.

In some embodiments, P₄ is a peptide sequence at least 5 amino acids in length. In some embodiments, P₄ is a peptide sequence at least 6 amino acids in length. In some embodiments, P₄ is a peptide sequence at least 10 amino acids in length. In some embodiments, P₄ is a peptide sequence at least 20 amino acids in length. In some embodiments, P₄ is a linear peptide. In some embodiments, P₄ is a cyclic peptide. In some embodiments, P₄ is resistant to cleavage by a protease while L₄ is cleavable by a tumor specific protease.

In some embodiments, P₄ is not a natural binding partner of T₃ or T₄. In some instances, P₄ is a modified binding partner of T₃ and T₄ and contains amino acid changes that at least slightly decrease affinity and/or avidity of binding to T₃ and T₄. In some embodiments, P₄ contains no or substantially no homology to T₃ and T₄ natural binding partner. In some embodiments, P₄ contains at least 5%, 10%, 15%, 20%, 25%, 30%, 35%, 40%, 45%, 50%, 55%, 60%, 65%, 70%, 75%, or 80% sequence identity to the natural binding partner of T₃ and T₄. In some embodiments, P₄ contains at least 5%, 10%, 15%, 20%, 25%, 30%, 35%, 40%, 45%, 50%, 55%, 60%, 65%, 70%, 75%, or 80% sequence identity to the target antigen.

In some embodiments, P₃ or P₄ or P₃ and P₄ are substrates for a tumor specific protease. In some embodiments, the tumor specific protease is a metalloprotease, serine protease, cysteine protease, threonine protease, and aspartic protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, P₃ or P₄ or P₃ and P₄ comprise a modified amino acid or non-natural amino acid, or a modified non-natural amino acid, or a combination thereof. In some embodiments, the modified amino acid or a modified non-natural amino acid comprises a post-translational modification. In some embodiments P₃ or P₄ or P₃ and P₄ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₃ or P₄ or P₃ and P₄ including the peptide backbone, the amino acid side chains, and the terminus.

Linking Moiety (L₃ and L₄)

In some embodiments, L₃ is cleavable by a protease. In some embodiments, L₃ is cleavable by a protease that is specific to a particular microenvironment. In some embodiments, L₃ is resistant to protease cleavage, while P₃ is cleavable by a protease. In some embodiments, the protease is a metalloprotease, serine protease, cysteine protease, threonine protease, and aspartic protease. In some embodiments, L₃ is cleavable by a tumor specific protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, L₄ is cleavable by a protease. In some embodiments, L₄ is cleavable by a protease that is specific to a particular microenvironment. In some embodiments, L₄ is resistant to protease cleavage, while P₂ is cleavable by a protease. In some embodiments, the protease is metalloprotease, serine protease, cysteine protease, threonine protease, and aspartic protease. In some embodiments, L₄ is cleavable by a tumor specific protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, L₃ is a peptide sequence having at least 5 to no more than 50 amino acids. In some embodiments, L₃ has a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₃ has a formula comprising (GGSGGD)ₙ, wherein n is an integer from 2 to 6 (SEQ ID NO: 8). In some embodiments, L₃ has a formula comprising (GGSGGE)ₙ, wherein n is an integer from 2 to 6 (SEQ ID NO: 9). In some embodiments, L₃ has a formula comprising (GGGSGSGGGGS)ₙ, wherein n is an integer from 1 to 3 (SEQ ID NO: 6). In some embodiments, L₃ has a formula comprising (GGGGGPGGGGP)ₙ, wherein n is an integer from 1 to 3 (SEQ ID NO: 7). In some embodiments, L₃ has a formula selected from: (GX)ₙ, wherein X is serine, aspartic acid, glutamic acid, threonine, or proline and n is at least 20 (SEQ ID NO: 24); (GGX)ₙ wherein X is serine, aspartic acid, glutamic acid, threonine, or proline and n is at least 13 (SEQ ID NO: 25); (GGGX)ₙ wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₃ comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₃ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₃ comprises an MMP cleavable amino acid sequence. In some embodiments, the MMP cleavable amino acid sequence is PLGLWA (SEQ ID NO: 19). In some embodiments, L₃ comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₃ comprises the sequence L₃ₓ-L_{3c}-L_{3z} wherein L_{ac} is a cleavable sequence. In some embodiments, L_{3c} comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₃ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L_{3c} comprises an MMP cleavable amino acid sequence. In some embodiments, the MMP cleavable amino acid sequence is PLGLWA (SEQ ID NO: 19). In some embodiments, L_{3c} comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₃ₓ or L_{3z} have a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₃ₓ or L_{3z} have a formula comprising (GGSGGD)ₙ, wherein n is an integer from 2 to 6 (SEQ ID NO: 8). In some embodiments, L₃ₓ or L_{3z} have a formula comprising (GGSGGE)ₙ, wherein n is an integer from 2 to 6 (SEQ ID NO: 9). In some embodiments, L₃ₓ or L_{3z} have a formula comprising (GGGSGSGGGGS)ₙ, wherein n is an integer from 1 to 3 (SEQ ID NO: 6). In some embodiments, L₃ₓ or L_{3z} have a formula comprising (GGGGGPGGGGP)ₙ, wherein n is an integer from 1 to 3 (SEQ ID NO: 7). In some embodiments, L₃ₓ or L_{3z} have a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₄ is a peptide sequence having at least 5 to no more than 50 amino acids. In some embodiments, L₄ has a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄5)ₙ, wherein n is an integer from 2 to 8 (SEQ ID NO: 4); and (G)ₙ, wherein n is an integer from 12 to 40 (SEQ ID NO: 5). In some embodiments, L₄ has a formula comprising (GGSGGD)ₙ, wherein n is an integer from 2 to 6 (SEQ ID NO: 8). In some embodiments, L₄ has a formula comprising (GGSGGE)ₙ, wherein n is an integer from 2 to 6 (SEQ ID NO: 9). In some embodiments, L₄ has a formula comprising (GGGSGSGGGGS)ₙ, wherein n is an integer from 1 to 3 (SEQ ID NO: 6). In some embodiments, L₄ has a formula comprising (GGGGGPGGGGP)ₙ, wherein n is an integer from 1 to 3 (SEQ ID NO: 7). In some embodiments, L₄ has a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₄ comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₄ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₄ comprises an MMP cleavable amino acid sequence. In some embodiments, the MMP cleavable amino acid sequence is PLGLWA (SEQ ID NO: 19). In some embodiments, L₄ comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₄ comprises the sequence L₄ₓ-L_{4c}-L_{4z} wherein Lₖ is a cleavable sequence. In some embodiments, L_{4c} comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L_{4c} comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L_{4c} comprises an MMP cleavable amino acid sequence. In some embodiments, the MMP cleavable amino acid sequence is PLGLWA (SEQ ID NO: 19). In some embodiments, L_{4c} comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₄ₓ or L_{4z} have a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₄ₓ or L_{4z} have a formula comprising (GGSGGD)ₙ, wherein n is an integer from 2 to 6 (SEQ ID NO: 8). In some embodiments, L₄ₓ or L_{4z} have a formula comprising (GGSGGE)ₙ, wherein n is an integer from 2 to 6 (SEQ ID NO: 9). In some embodiments, L₄ₓ or L_{4z} have a formula comprising (GGGSGSGGGGS)ₙ, wherein n is an integer from 1 to 3 (SEQ ID NO: 6). In some embodiments, L₄ₓ or L_{4z} have a formula comprising (GGGGGPGGGGP)ₙ, wherein n is an integer from 1 to 3 (SEQ ID NO: 7). In some embodiments, L₄ₓ or L_{4z} have a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₃ or L₄ or L₃ and L₄ comprise a modified amino acid or non-natural amino acid, or a modified non-natural amino acid, or a combination thereof. In some embodiments, the modified amino acid or a modified non-natural amino acid comprises a post-translational modification. In some embodiments, L₃ or L₄ or L₃ and L₄ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₃ or L₄ or L₃ and L₄ including the peptide backbone, or the amino acid side chains.

### TCR Alpha Extracellular Domain or a TCR Beta Extracellular Domain (T₃ and T)

In some embodiments, the TCR alpha extracellular domain, or fragment thereof, comprises a variable region. In some embodiments, the TCR alpha extracellular domain, or fragment thereof comprises a variable region, a joining region, and a constant region. In some embodiments, the TCR alpha extracellular domain, or fragment thereof, comprises three hyper-variable complementarity determining regions (CDRs) within the variable region. In some embodiments, at least one CDR comprises a mutation to increase binding affinity or binding specificity to the target antigen or to increase binding affinity and binding specificity to the target antigen. In some embodiments, there are 2-20, 3-15, 4-12, or 4-10 mutations in one or two CDRs.

In some embodiments, the TCR alpha extracellular domain, or fragment thereof, comprises a modified amino acid. In some embodiments, the modified amino acid comprises a post-translational modification. In some embodiments, the TCR alpha extracellular domain, or fragment thereof, comprises a non-natural amino acid or a modified non-natural amino acid, or combination thereof. In some embodiments, the modified non-natural amino acid comprises a post-translational modification.

In some embodiments, the TCR beta extracellular domain, or fragment thereof comprises a variable region. In some embodiments, the TCR beta extracellular domain, or fragment thereof comprises a variable region, a joining region, and a constant region. In some embodiments, the TCR beta extracellular domain or fragment thereof, comprises three hyper-variable complementarity determining regions (CDRs). In some embodiments, at least one CDR comprises a mutation to increase binding affinity or binding specificity to the target antigen or to increase binding affinity and binding specificity to the target antigen. In some embodiments, there are 2-20, 3-15, 4-12, or 4-10 mutations in one or two CDRs.

In some embodiments, the TCR beta extracellular domain or fragment thereof, comprises a modified amino acid. In some embodiments, the modified amino acid comprises a post-translational modification. In some embodiments, the TCR beta extracellular domain, or fragment thereof, comprises a non-natural amino acid or a modified non-natural amino acid, or combination thereof. In some embodiments, the modified non-natural amino acid comprises a post-translational modification.

In some embodiments, the TCR alpha extracellular domain, or fragment thereof, comprises a truncated transmembrane domain. In some embodiments, the TCR beta extracellular domain comprises a truncated transmembrane domain.

In some embodiments, the TCR alpha extracellular domain, or fragment thereof, and the TCR beta extracellular domain, or fragment thereof, are connected by a disulfide bond. In some embodiments, the TCR alpha extracellular domain comprises an alpha chain TRAC constant domain sequence and the TCR beta extracellular domain comprises a beta chain TRBC1 or TRBC2 constant domain sequence. In some embodiments, Cys4 of the alpha chain TRAC constant domain sequence is modified by truncation or substitution and Cys2 of exon 2 of the beta chain TRBC1 or TRBC2 constant domain sequence is modified by truncation or substitution, thereby deleting a native disulfide bond. In some embodiments, Thr48 of the alpha chain TRAC constant domain sequence is mutated to Cys and Ser57 of the beta chain TRBC1 or TRBC2 constant domain sequence is mutated to Cys.

In some embodiments, the TCR alpha extracellular domain, or fragment thereof, further comprises an effector domain. In some embodiments, the TCR beta extracellular domain, or fragment thereof, further comprises an effector domain.

In some embodiments, the modified TCR heterodimer comprises an effector domain. In some embodiments, the effector domain is an anti-CD3 moiety. In some embodiments, the TCR alpha extracellular domain or the TCR beta extracellular domain is bound to an anti-CD3 single-chain variable fragment (scFv) effector. In some embodiments, the TCR alpha extracellular domain or the TCR beta extracellular domain is bound to an Fc that is also bound to an anti-CD3 scFv.

### Soluble, Single-Chain Modified TCRs

Disclosed herein, in certain embodiments, are modified T cell receptors (TCR) comprising a polypeptide of formula V:

T₅-L₅-P₅ (formula V)

wherein T₅ comprises a variable region of a TCR alpha extracellular domain, or fragment thereof, and a variable region of a TCR beta extracellular domain, or fragment thereof, wherein T₅ binds to a target antigen, and the variable region of TCR alpha extracellular domain, or fragment thereof, and the variable region of the TCR beta extracellular domain, or fragment thereof contain an antigen binding site, P₅ is a peptide that reduces binding of T₅ to the target antigen when the modified TCR is outside of a tumor microenvironment and that does not reduce binding of T₅ to the target antigen when the modified TCR is inside the tumor microenvironment, and L₅ is a linking moiety that connects T₅ to P₅ and L₅ is bound to T₅ at the N-terminus of T₅, wherein the modified TCR is a soluble TCR and is a functional TCR when inside the tumor microenvironment and is a nonfunctional TCR when outside the tumor microenvironment and P₅ or L₅ is a substrate for a tumor specific protease.

In some embodiments, the target antigen includes, but is not limited to MAGE-A3, NY-ESO-1, gp100, WT1, and tyrosinase. In some embodiments, the target antigen is MAGE-A3. In some embodiments, the target antigen is NY-ESO-1. In some embodiments, the target antigen is gp100. In some embodiments, the target antigen is WT1. In some embodiments, the target antigen is tyrosinase.

### Peptide (P₅)

In some embodiments, P₅ binds to T₅ thereby concealing the antigen binding site of T₅ from engaging with the target antigen. In some embodiments, P₅ binds to T₅ when the modified TCR is outside of a tumor microenvironment. In some embodiments, when the modified TCR is inside the tumor microenvironment, P₅ is cleaved from the polypeptide chain, thereby exposing the antigen binding sites of T₅.

In some embodiments, P₅ is bound to T₅ through ionic interactions, electrostatic interactions, hydrophobic interactions, Pi-stacking interactions, and H-bonding interactions, or a combination thereof when the modified TCR is outside the tumor microenvironment. In some embodiments, P₅ is bound to T₅ at or near the antigen binding site when the modified TCR is outside the tumor microenvironment. In some embodiments, P₅ inhibits the binding of T₃ to the target antigen when the modified TCR is outside the tumor microenvironment, and P₃ does not inhibit the binding of T₅ to the target antigen when the modified TCR is inside the tumor microenvironment. In some embodiments, P₅ sterically blocks T₃ from binding to the target antigen when the modified TCR is outside the tumor microenvironment. In some embodiments, P₅ is removed from the antigen binding site, and the antigen binding site of T₅ is exposed when the modified TCR is inside the tumor microenvironment.

In some embodiments, P₅ is a peptide sequence at least 5 amino acids in length. In some embodiments, P₅ is a peptide sequence at least 6 amino acids in length. In some embodiments, P₅ is a peptide sequence at least 10 amino acids in length. In some embodiments, P₅ is a peptide sequence at least 20 amino acids in length. In some embodiments, P₅ is a linear peptide. In some embodiments, P₅ is a cyclic peptide. In some embodiments, P₅ is resistant to cleavage by a protease while L₅ is cleavable by a tumor specific protease.

In some embodiments, P₅ is not a natural binding partner of T₅. In some instances, P₅ is a modified binding partner of T₅ and contains amino acid changes that at least slightly decrease affinity and/or avidity of binding to T₅. In some embodiments, P₅ contains no or substantially no homology to T₅ natural binding partner. In some embodiments, P₅ contains at least 5%, 10%, 15%, 20%, 25%, 30%, 35%, 40%, 45%, 50%, 55%, 60%, 65%, 70%, 75%, or 80% sequence identity to the natural binding partner of T₅. In some embodiments, P₅ contains at least 5%, 10%, 15%, 20%, 25%, 30%, 35%, 40%, 45%, 50%, 55%, 60%, 65%, 70%, 75%, or 80% sequence identity to the natural binding partner of T₅. In some embodiments, P₅ contains at least 5%, 10%, 15%, 20%, 25%, 30%, 35%, 40%, 45%, 50%, 55%, 60%, 65%, 70%, 75%, or 80% sequence identity to the target antigen.

In some embodiments, P₅ is a substrate for a tumor specific protease. In some embodiments, the tumor specific protease is a metalloprotease, serine protease, cysteine protease, threonine protease, and aspartic protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

In some embodiments, P₅ comprises a modified amino acid or non-natural amino acid, or a modified non-natural amino acid, or a combination thereof. In some embodiments, the modified amino acid or a modified non-natural amino acid comprises a post-translational modification. In some embodiments P₅ comprises a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₅ including the peptide backbone, the amino acid side chains, and the terminus.

### Linking Moiety (L₅)

In some embodiments, L₅ is cleavable by a protease. In some embodiments, L₅ is cleavable by a protease that is specific to a particular microenvironment. In some embodiments, L₅ is resistant to protease cleavage, while P₅ is cleavable by a protease. In some embodiments, the protease is a metalloprotease, serine protease, cysteine protease, threonine protease, and aspartic protease. In some embodiments, L₅ is cleavable by a tumor specific protease. In some embodiments, the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

L₅ is a peptide sequence having at least 5 to no more than 50 amino acids. L₅ has a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). L₅ has a formula comprising (GGSGGD)ₙ, wherein n is an integer from 2 to 6 (SEQ ID NO: 8). L₅ has a formula comprising (GGSGGE)ₙ, wherein n is an integer from 2 to 6 (SEQ ID NO: 9). L₅ has a formula comprising (GGGSGSGGGGS)ₙ, wherein n is an integer from 1 to 3 (SEQ ID NO: 6). L₅ has a formula comprising (GGGGGPGGGGP)ₙ, wherein n is an integer from 1 to 3 (SEQ ID NO: 7). L₅ has a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₅ comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₅ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₅ comprises an MMP cleavable amino acid sequence. In some embodiments, the MMP cleavable amino acid sequence is PLGLWA (SEQ ID NO: 19). In some embodiments, L₅ comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₅ comprises the sequence L₅ₓ-L_{5c}-L_{5z} wherein L_{5c} is a cleavable sequence. In some embodiments, L_{5c} comprises a plasmin cleavable amino acid sequence. In some embodiments, the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15). In some embodiments, L₅ comprises a Factor Xa cleavable amino acid sequence. In some embodiments, the Factor Xa cleavable amino acid sequence is selected from the group consisting of IEGR (SEQ ID NO: 16), IDGR (SEQ ID NO: 17), and GGSIDGR (SEQ ID NO: 18). In some embodiments, L₅ comprises an MMP cleavable amino acid sequence. In some embodiments, the MMP cleavable amino acid sequence is PLGLWA (SEQ ID NO: 19). In some embodiments, L_{5c} comprises a collagenase cleavable amino acid sequence. In some embodiments, the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

In some embodiments, L₅ₓ or L_{5z} have a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5). In some embodiments, L₅ₓ or L_{5z} have a formula comprising (GGSGGD)ₙ, wherein n is an integer from 2 to 6 (SEQ ID NO: 8). In some embodiments, L₅ₓ or L_{5z} have a formula comprising (GGSGGE)ₙ, wherein n is an integer from 2 to 6 (SEQ ID NO: 9). In some embodiments, L₅ₓ or L_{5z} have a formula comprising (GGGSGSGGGGS)ₙ, wherein n is an integer from 1 to 3 (SEQ ID NO: 6). In some embodiments, L₅ₓ or L_{5z} have a formula comprising (GGGGGPGGGGP)ₙ, wherein n is an integer from 1 to 3 (SEQ ID NO: 7). In some embodiments, L₅ₓ or L_{5z} have a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

In some embodiments, L₅ comprises a modified amino acid or non-natural amino acid, or a modified non-natural amino acid, or a combination thereof. In some embodiments, the modified amino acid or a modified non-natural amino acid comprises a post-translational modification. In some embodiments, L₅ comprises a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₅ including the peptide backbone, or the amino acid side chains.

### Variable Region of a TCR Alpha Extracellular Domain and a Variable Region of a TCR Beta Extracellular Domain (T)

In some embodiments, T₅ comprises a formula, Vα-L₅₁-Vβ, wherein Va is the variable region of the TCR alpha extracellular domain, or a fragment thereof, Vβ is the variable region of the TCR beta extracellular domain, or fragment thereof, and L₅₁ is a sequence that connects Vα and Vβ, wherein Vα is N-terminal to L₅₁. In some embodiments, T₅ comprises a formula Vβ-L₅₂-Vα wherein Vβ is the variable region of the TCR beta extracellular domain, or fragment thereof, Vα is the variable region of the TCR alpha extracellular domain, or fragment thereof, and L₅₂ is a sequence that connects Vβ and Vα, wherein Vβ is N-terminal to L₅₂. In some embodiments, T₅ comprises a formula: Vα-L₅₃-Vβ-Cβ wherein Vα is the variable region of the TCR alpha extracellular domain, or fragment thereof, Vβ is the variable region of the TCR beta extracellular domain, or fragment thereof, Cβ is a constant region of the TCR beta extracellular domain, or a fragment thereof, and L₅₃ is a sequence that connects Vα and Vβ, wherein Vα is N-terminal to L₅₃. In some embodiments, T₅ comprises a formula Vβ-Cβ-L₅₄-Vα wherein Vβ is the variable region of the TCR beta extracellular domain, or a fragment thereof, Cβ is a constant region of the TCR beta extracellular domain, or a fragment thereof Vα is the variable region of the TCR alpha extracellular domain, or a fragment thereof, and L₅₄ is a sequence that connects Cβ and Vα, wherein Vβ is N-terminal to L₅₄. In some embodiments, T₅ comprises a formula Vα-Cα-L₅₅-Vβ wherein Vα is the variable region of the TCR alpha extracellular domain, or a fragment thereof, Cα is a constant region of the TCR alpha extracellular domain, or a fragment thereof, Vβ is the variable region of the TCR beta extracellular domain or a fragment thereof, and L₅₅ is a sequence that connects Cα and Vβ, wherein Vα is N-terminal to L₅₅. In some embodiments, T₅ comprises a formula Vβ-L₅₆-Vα-Cα wherein Vβ is the variable region of the TCR beta extracellular domain, or a fragment thereof, Vα is the variable region of the TCR alpha extracellular domain, or a fragment thereof, Cα is a constant region of the TCR alpha extracellular domain, or a fragment thereof, and L₅₆ is a sequence that connects Vβ and Vα, wherein Vβ is N-terminal to L₅₆. In some embodiments, the TCR alpha extracellular domain comprises three hyper-variable complementarity determining regions (CDRs).

In some embodiments, at least one CDR comprises a mutation to increase binding affinity or binding specificity to the target antigen or to increase binding affinity and binding specificity to the target antigen. In some embodiments, the variable region of the TCR alpha extracellular domain, or fragment thereof, comprises a modified amino acid. In some embodiments, the modified amino acid comprises a post-translational modification. In some embodiments, the variable region of the TCR alpha extracellular domain, or fragment thereof, comprises a non-natural amino acid or a modified non-natural amino acid, or combination thereof. In some embodiments, the modified non-natural amino acid comprises a post-translational modification. In some embodiments, the variable region of the TCR beta extracellular domain, or fragment thereof, comprises three hyper-variable complementarity determining regions (CDRs). In some embodiments, at least one CDR comprises a mutation to increase binding affinity or binding specificity to the target antigen or to increase binding affinity and binding specificity to the target antigen. In some embodiments, the variable region of the TCR beta extracellular domain, or fragment thereof, comprises a modified amino acid. In some embodiments, the modified amino acid comprises a post-translational modification. In some embodiments, the variable region of the TCR beta extracellular domain, or fragment thereof, comprises a non-natural amino acid or a modified non-natural amino acid, or combination thereof. In some embodiments, the modified non-natural amino acid comprises a post-translational modification. In some embodiments, T₅ further comprises a truncated transmembrane domain.

In some embodiments, T₅ comprises an effector domain. In some embodiments, T₅ comprises an effector domain. In some embodiments, the effector domain is an anti-CD3 moiety. In some embodiments, T₅ is bound to an anti-CD3 single-chain variable fragment (scFv) effector. In some embodiments, T₅ is bound to an Fc that is also bound to an anti-CD3 single-chain variable fragment (scFv) effector.

### Polynucleotides Encoding Polypeptides of Modified T Cell Receptors

Disclosed herein, in certain embodiments, are isolated recombinant nucleic acid molecules encoding modified T cell receptors (TCRs) as disclosed herein.

Disclosed herein, in certain embodiments, are isolated recombinant nucleic acid molecules encoding modified T cell receptors (TCRs) comprising a polypeptide of formula I:

T₁-L₁-P₁ (formula I)

wherein T₁ comprises a transmembrane domain and either a TCR alpha extracellular domain, or fragment thereof, or a TCR beta extracellular domain, or fragment thereof, wherein T₁ binds to a target antigen and the TCR alpha extracellular domain or fragment thereof and the TCR beta extracellular domain, or fragment thereof contain an antigen binding site, P₁ is a peptide that reduces binding of T₁ to the target antigen when the modified TCR is outside of a tumor microenvironment and that does not reduce binding of T₁ to the target antigen when the modified TCR is inside the tumor microenvironment, and L₁ is a linking moiety that connects T₁ to P₁ and L₁ is bound to T₁ at the N-terminus of T₁, wherein the modified TCR is a functional TCR when inside the tumor microenvironment and is a nonfunctional TCR when outside the tumor microenvironment and P₁ or L₁ is a substrate for a tumor specific protease. In some embodiments, T₁ comprises the TCR alpha extracellular domain, or fragment thereof, and the modified TCR further comprises a second polypeptide comprising a transmembrane domain and a TCR beta extracellular domain, or fragment thereof wherein the TCR beta extracellular domain or fragment thereof contains an antigen binding site. In some embodiments, T₁ comprises the TCR beta extracellular domain, or fragment thereof, and the modified TCR further comprises a second polypeptide comprising a transmembrane domain and a TCR alpha extracellular domain, or fragment thereof wherein the TCR alpha extracellular domain or fragment thereof contains an antigen binding site. In some embodiments, T₁ comprises the TCR alpha extracellular domain, or fragment thereof, and the modified TCR further comprises a second polypeptide of formula II:

T₂-L₂-P₂ (formula II)

wherein T₂ comprises a transmembrane domain and a TCR beta extracellular domain, or fragment thereof, wherein T₂ binds to the target antigen and the TCR beta extracellular domain or fragment thereof contains an antigen binding site, P₂ is a peptide that reduces binding of T₂ to the target antigen when the modified TCR is outside of a tumor microenvironment and that does not reduce binding of T₂ to the target antigen when the modified TCR is inside the tumor microenvironment, and L₂ is a linking moiety that connects T₂ to P₂ and L₂ is bound to T₂ at the N-terminus of T₂, wherein P₂ or L₂ is a substrate for a tumor specific protease. In some embodiments, the polypeptide of formula I and formula II are expressed from the same plasmid. In some embodiments, the polypeptide of formula I and formula II are expressed from separate plasmids.

Disclosed herein, in certain embodiments, are isolated recombinant nucleic acid molecules encoding modified T cell receptors (TCRs) comprising a polypeptide of formula III:

T₃-L₃-P₃ (formula III)

wherein T₃ comprises either a TCR alpha extracellular domain, or fragment thereof, or a TCR beta extracellular domain, or fragment thereof, wherein T₃ binds to a target antigen and the TCR alpha extracellular domain or fragment thereof and the TCR beta extracellular domain, or fragment thereof contain an antigen binding site, P₃ is a peptide that reduces binding of T₃ to the target antigen when the modified TCR is outside of a tumor microenvironment and that does not reduce binding of T₃ to the target antigen when the modified TCR is inside the tumor microenvironment, and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease. In some embodiments, T₃ comprises the TCR alpha extracellular domain, or fragment thereof, and the modified TCR further comprises a second polypeptide comprising a TCR beta extracellular domain, or fragment thereof wherein the TCR beta extracellular domain or fragment thereof contains an antigen binding site. In some embodiments, T₃ comprises the TCR beta extracellular domain, or fragment thereof, and the modified TCR further comprises a second polypeptide comprising a TCR alpha extracellular domain, or fragment thereof wherein the TCR alpha extracellular domain or fragment thereof contains an antigen binding site. In some embodiments, the T₃ comprises the TCR alpha extracellular domain, or fragment thereof, and the modified TCR further comprises a second polypeptide of formula IV:

T₄-L₄-P₄ (formula IV)

wherein T₄ comprises a TCR beta extracellular domain, or fragment thereof, wherein T₄ binds to the target antigen and the TCR beta extracellular domain or fragment thereof contains an antigen binding site, P₄ is a peptide that reduces binding of T₄ to the target antigen when the modified TCR is outside of a tumor microenvironment and that does not reduce binding of T₄ to the target antigen when the modified TCR is inside the tumor microenvironment, and L₄ is a linking moiety that connects T₄ to P₄ and L₄ is bound to T₄ at the N-terminus of T₄, wherein P₂ or L₂ is a substrate for a tumor specific protease. In some embodiments, the polypeptide of formula III and formula IV are expressed from the same plasmid. In some embodiments, the polypeptide of formula III and formula IV are expressed from separate plasmids.

Disclosed herein, in certain embodiments, are isolated recombinant nucleic acid molecules encoding modified T cell receptors (TCR) comprising a polypeptide of formula V:

T₅-L₅-P₅ (formula V)

wherein T₅ comprises a variable region of a TCR alpha extracellular domain, or fragment thereof, and a variable region of a TCR beta extracellular domain, or fragment thereof, wherein T₅ binds to a target antigen and the variable region of TCR alpha extracellular domain, or fragment thereof, and the variable region of the TCR beta extracellular domain, or fragment thereof contain an antigen binding site, P₅ is a peptide that reduces binding of T₅ to the target antigen when the modified TCR is outside of a tumor microenvironment and that does not reduce binding of T₅ to the target antigen when the modified TCR is inside the tumor microenvironment, and L₅ is a linking moiety that connects T₅ to P₅ and L₅ is bound to T₅ at the N-terminus of T₅, wherein the modified TCR is a soluble TCR and is a functional TCR when inside the tumor microenvironment and is a nonfunctional TCR when outside the tumor microenvironment and P₅ or L₅ is a substrate for a tumor specific protease. In some embodiments, T₅ comprises a formula:

Vα-L₅₁-Vβ

wherein Vα is the variable region of the TCR alpha extracellular domain, or fragment thereof, Vβ is the variable region of the TCR beta extracellular domain, or fragment thereof, and L₅₁ is a sequence that connects Vα and Vβ, wherein Vα is N-terminal to L₅₁. In some embodiments, T₅ comprises a formula:

Vβ-L₅₂-Vα

wherein Vβ is the variable region of the TCR beta extracellular domain, or fragment thereof, Vα is the variable region of the TCR alpha extracellular domain, or fragment thereof, and L₅₂ is a sequence that connects Vβ and Vα, wherein Vβ is N-terminal to L₅₂. In some embodiments, T₅ comprises a formula:

Vα-L₅₃-Vβ−Cβ

wherein Vα is the variable region of the TCR alpha extracellular domain, or fragment thereof, Vβ is the variable region of the TCR beta extracellular domain, or fragment thereof, Cβ is a constant region of the TCR beta extracellular domain, or fragment thereof, and L₅₃ is a sequence that connects Vα and Vβ, wherein Vα is N-terminal to L₅₃. In some embodiments, T₅ comprises a formula:

Vβ−Cβ-L₅₄-Vα

wherein Vβ is the variable region of the TCR beta extracellular domain, or fragment thereof, Cβ is a constant region of the TCR beta extracellular domain, or fragment thereof, Vα is the variable region of the TCR alpha extracellular domain, or fragment thereof, and L₅₄ is a sequence that connects Cβ and Vα, wherein Vβ is N-terminal to L₅₄. In some embodiments, T₅ comprises a formula:

Vα−Cα-L₅₅-Vβ

wherein Vα is the variable region of the TCR alpha extracellular domain, or fragment thereof, Cα is a constant region of the TCR alpha extracellular domain, or fragment thereof, Vβ is the variable region of the TCR beta extracellular domain, or fragment thereof, and L₅₅ is a sequence that connects Cα and Vβ, wherein Vα is N-terminal to L₅₅. In some embodiments, T₅ comprises a formula:

Vβ-L₅₆-Vα−Cα

wherein Vβ is the variable region of the TCR beta extracellular domain, or fragment thereof, Vα is the variable region of the TCR alpha extracellular domain, or fragment thereof, Cα is a constant region of the TCR alpha extracellular domain, or fragment thereof, and L₅₆ is a sequence that connects Vβ and Vα, wherein Vβ is N-terminal to L₅₆.

In some embodiments, the isolated recombinant nucleic acid molecules encoding modified T cell receptors (TCRs) are provided as a DNA construct. In other embodiments, the isolated recombinant nucleic acid molecules encoding modified T cell receptors (TCRs) are provided as a messenger RNA transcript.

The polynucleotide molecules are constructed by known methods such as by combining the genes encoding the domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotide in the respective host cell.

In some embodiments, the nucleic acid molecule encoding a modified TCR disclosed herein is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described antigen-binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the modified TCRs as described herein, in some embodiments, are produced by introducing a vector encoding the polypeptides described above into a host cell and culturing said host cell under conditions whereby the protein domains are expressed, may be isolated and, optionally, further purified.

### Pharmaceutical Compositions

Disclosed herein, in certain embodiments, are pharmaceutical compositions comprising: (a) modified T cell receptors (TCRs) as disclosed herein; and (b) a pharmaceutically acceptable carrier or excipient.

In some embodiments, a pharmaceutical composition disclosed herein comprises (a) a modified T cell receptors (TCR) comprising a polypeptide of formula I:

T₁-L₁-P₁ (formula I)

wherein T₁ comprises a transmembrane domain and either a TCR alpha extracellular domain, or fragment thereof, or a TCR beta extracellular domain, or fragment thereof, wherein T₁ binds to a target antigen and the TCR alpha extracellular domain or fragment thereof and the TCR beta extracellular domain, or fragment thereof contain an antigen binding site, P₁ is a peptide that reduces binding of T₁ to the target antigen when the modified TCR is outside of a tumor microenvironment and that does not reduce binding of T₁ to the target antigen when the modified TCR is inside the tumor microenvironment, and L₁ is a linking moiety that connects T₁ to P₁ and L₁ is bound to T₁ at the N-terminus of T₁, wherein the modified TCR is a functional TCR when inside the tumor microenvironment and is a nonfunctional TCR when outside the tumor microenvironment and P₁ or L₁ is a substrate for a tumor specific protease. In some embodiments, T₁ comprises the TCR alpha extracellular domain, or fragment thereof, and the modified TCR further comprises a second polypeptide comprising a transmembrane domain and a TCR beta extracellular domain, or fragment thereof wherein the TCR beta extracellular domain or fragment thereof contains an antigen binding site. In some embodiments, T₁ comprises the TCR beta extracellular domain, or fragment thereof, and the modified TCR further comprises a second polypeptide comprising a transmembrane domain and a TCR alpha extracellular domain, or fragment thereof wherein the TCR alpha extracellular domain or fragment thereof contains an antigen binding site. In some embodiments, T₁ comprises the TCR alpha extracellular domain, or fragment thereof, and the modified TCR further comprises a second polypeptide of formula II:

T₂-L₂-P₂ (formula II)

wherein T₂ comprises a transmembrane domain and a TCR beta extracellular domain, or fragment thereof, wherein T₂ binds to the target antigen and the TCR beta extracellular domain or fragment thereof contains an antigen binding site, P₂ is a peptide that reduces binding of T₂ to the target antigen when the modified TCR is outside of a tumor microenvironment and that does not reduce binding of T₂ to the target antigen when the modified TCR is inside the tumor microenvironment, and L₂ is a linking moiety that connects T₂ to P₂ and L₂ is bound to T₂ at the N-terminus of T₂, wherein P₂ or L₂ is a substrate for a tumor specific protease.

In some embodiments, a pharmaceutical composition disclosed herein comprises (a) a modified T cell receptors (TCR) comprising a polypeptide of formula I:

T₃-L₃-P₃ (formula III)

wherein T₃ comprises either a TCR alpha extracellular domain, or fragment thereof, or a TCR beta extracellular domain, or fragment thereof, wherein T₃ binds to a target antigen and the TCR alpha extracellular domain or fragment thereof and the TCR beta extracellular domain, or fragment thereof contain an antigen binding site, P₃ is a peptide that reduces binding of T₃ to the target antigen when the modified TCR is outside of a tumor microenvironment and that does not reduce binding of T₃ to the target antigen when the modified TCR is inside the tumor microenvironment, and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease. In some embodiments, T₃ comprises the TCR alpha extracellular domain, or fragment thereof, and the modified TCR further comprises a second polypeptide comprising a TCR beta extracellular domain, or fragment thereof wherein the TCR beta extracellular domain or fragment thereof contains an antigen binding site. In some embodiments, T₃ comprises the TCR beta extracellular domain, or fragment thereof, and the modified TCR further comprises a second polypeptide comprising a TCR alpha extracellular domain, or fragment thereof wherein the TCR alpha extracellular domain or fragment thereof contains an antigen binding site. In some embodiments, the T₃ comprises the TCR alpha extracellular domain, or fragment thereof, and the modified TCR further comprises a second polypeptide of formula IV:

T₄-L₄-P₄ (formula IV)

wherein T₄ comprises a TCR beta extracellular domain, or fragment thereof, wherein T₄ binds to the target antigen and the TCR beta extracellular domain or fragment thereof contains an antigen binding site, P₄ is a peptide that reduces binding of T₄ to the target antigen when the modified TCR is outside of a tumor microenvironment and that does not reduce binding of T₄ to the target antigen when the modified TCR is inside the tumor microenvironment, and L₄ is a linking moiety that connects T₄ to P₄ and L₄ is bound to T₄ at the N-terminus of T₄, wherein P₂ or L₂ is a substrate for a tumor specific protease.

In some embodiments, a pharmaceutical composition disclosed herein comprises (a) a modified T cell receptors (TCR) comprising a polypeptide of formula I:

T₅-L₅-P₅ (formula V)

wherein T₅ comprises a variable region of a TCR alpha extracellular domain, or fragment thereof, and a variable region of a TCR beta extracellular domain, or fragment thereof, wherein T₅ binds to a target antigen and the variable region of TCR alpha extracellular domain, or fragment thereof, and the variable region of the TCR beta extracellular domain, or fragment thereof contain an antigen binding site, P₅ is a peptide that reduces binding of T₅ to the target antigen when the modified TCR is outside of a tumor microenvironment and that does not reduce binding of T₅ to the target antigen when the modified TCR is inside the tumor microenvironment, and L₅ is a linking moiety that connects T₅ to P₅ and L₅ is bound to T₅ at the N-terminus of T₅, wherein the modified TCR is a soluble TCR and is a functional TCR when inside the tumor microenvironment and is a nonfunctional TCR when outside the tumor microenvironment and P₅ or L₅ is a substrate for a tumor specific protease. In some embodiments, T₅ comprises a formula:

Vα-L₅₁-Vβ

wherein Vα is the variable region of the TCR alpha extracellular domain, or fragment thereof, Vβ is the variable region of the TCR beta extracellular domain, or fragment thereof, and L₅₁ is a sequence that connects Vα and Vβ, wherein Vα is N-terminal to L₅₁. In some embodiments, T₅ comprises a formula:

Vβ-L₅₂-Vα

wherein Vβ is the variable region of the TCR beta extracellular domain, or fragment thereof, Vα is the variable region of the TCR alpha extracellular domain, or fragment thereof, and L₅₂ is a sequence that connects Vβ and Vα, wherein Vβ is N-terminal to L₅₂. In some embodiments, T₅ comprises a formula:

Vα-L₅₃-Vβ−Cβ

wherein Vα is the variable region of the TCR alpha extracellular domain, or fragment thereof, Vβ is the variable region of the TCR beta extracellular domain, or fragment thereof, Cβ is a constant region of the TCR beta extracellular domain, or fragment thereof, and L₅₃ is a sequence that connects Vα and Vβ, wherein Vα is N-terminal to L₅₃. In some embodiments, T₅ comprises a formula:

Vβ−Cβ-L₅₄-Vα

wherein Vβ is the variable region of the TCR beta extracellular domain, or fragment thereof, Cβ is a constant region of the TCR beta extracellular domain, or fragment thereof, Vα is the variable region of the TCR alpha extracellular domain, or fragment thereof, and L₅₄ is a sequence that connects Cβ and Vα, wherein Vβ is N-terminal to L₅₄. In some embodiments, T₅ comprises a formula:

Vα−Cα-L₅₅-Vβ

wherein Vα is the variable region of the TCR alpha extracellular domain, or fragment thereof, Cα is a constant region of the TCR alpha extracellular domain, or fragment thereof, Vβ is the variable region of the TCR beta extracellular domain, or fragment thereof, and L₅₅ is a sequence that connects Cα and Vβ, wherein Vα is N-terminal to L₅₅. In some embodiments, T₅ comprises a formula:

Vβ-L₅₆-Vα−Cα

wherein Vβ is the variable region of the TCR beta extracellular domain, or fragment thereof, Vα is the variable region of the TCR alpha extracellular domain, or fragment thereof, Cα is a constant region of the TCR alpha extracellular domain, or fragment thereof, and L₅₆ is a sequence that connects Vβ and Vα, wherein Vβ is N-terminal to L₅₆.

In some embodiments, a pharmaceutical composition disclosed herein comprises an isolated recombinant nucleic acid molecule encoding modified T cell receptors (TCRs) comprising a polypeptide of formula I:

T₁-L₁-P₁ (formula I)

wherein T₁ comprises a transmembrane domain and either a TCR alpha extracellular domain, or fragment thereof, or a TCR beta extracellular domain, or fragment thereof, wherein T₁ binds to a target antigen and the TCR alpha extracellular domain or fragment thereof and the TCR beta extracellular domain, or fragment thereof contain an antigen binding site, P₁ is a peptide that reduces binding of T₁ to the target antigen when the modified TCR is outside of a tumor microenvironment and that does not reduce binding of T₁ to the target antigen when the modified TCR is inside the tumor microenvironment, and L₁ is a linking moiety that connects T₁ to P₁ and L₁ is bound to T₁ at the N-terminus of T₁, wherein the modified TCR is a functional TCR when inside the tumor microenvironment and is a nonfunctional TCR when outside the tumor microenvironment and P₁ or L₁ is a substrate for a tumor specific protease.

In some embodiments, a pharmaceutical composition disclosed herein comprises an isolated recombinant nucleic acid molecule encoding modified T cell receptors (TCRs) comprising a polypeptide of formula III:

T₃-L₃-P₃ (formula III)

wherein T₃ comprises either a TCR alpha extracellular domain, or fragment thereof, or a TCR beta extracellular domain, or fragment thereof, wherein T₃ binds to a target antigen and the TCR alpha extracellular domain or fragment thereof and the TCR beta extracellular domain, or fragment thereof contain an antigen binding site, P₃ is a peptide that reduces binding of T₃ to the target antigen when the modified TCR is outside of a tumor microenvironment and that does not reduce binding of T₃ to the target antigen when the modified TCR is inside the tumor microenvironment, and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease.

In some embodiments, a pharmaceutical composition disclosed herein comprises an isolated recombinant nucleic acid molecule encoding modified T cell receptors (TCRs) comprising a polypeptide of formula V:

T₅-L₅-P₅ (formula V)

wherein T₅ comprises a variable region of a TCR alpha extracellular domain, or fragment thereof, and a variable region of a TCR beta extracellular domain, or fragment thereof, wherein T₅ binds to a target antigen and the variable region of TCR alpha extracellular domain, or fragment thereof, and the variable region of the TCR beta extracellular domain, or fragment thereof contain an antigen binding site, P₅ is a peptide that reduces binding of T₅ to the target antigen when the modified TCR is outside of a tumor microenvironment and that does not reduce binding of T₅ to the target antigen when the modified TCR is inside the tumor microenvironment, and L₅ is a linking moiety that connects T₅ to P₅ and L₅ is bound to T₅ at the N-terminus of T₅, wherein the modified TCR is a soluble TCR and is a functional TCR when inside the tumor microenvironment and is a nonfunctional TCR when outside the tumor microenvironment and P₅ or L₅ is a substrate for a tumor specific protease.

In some embodiments, the modified TCR further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the TCRs as described herein (as a soluble TCR or expressed on a transfected T-cell), may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Soluble TCRs, or cells, in accordance with the invention will usually be supplied as part of a sterile, pharmaceutical composition which will normally include a pharmaceutically acceptable carrier. This pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

### T Cell Preparation and Expansion

In some embodiments, the modified TCRs described herein are introduced into a cytotoxic cell. In some embodiments, the cytotoxic cell is a T cell. In some embodiments, the T cell is a naïve T cell, a central memory cell, or an effector memory T cell.

### Sources of T Cells

In some embodiments, a source of T-cells is obtained from a subject. The term “subject” is intended to include living organisms in which an immune response can be elicited (e.g., mammals). T-cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, T-cells can be obtained from a unit of blood collected from a subject using any number of techniques known to the skilled artisan, such as density gradient centrifugation using Ficoll related medium separation. In one embodiment, cells from the circulating blood of an individual are obtained by apheresis. The apheresis product typically contains lymphocytes, including T-cells, monocytes, granulocytes, B cells, other nucleated white blood cells, red blood cells, and platelets. In one embodiment, the cells collected by apheresis are washed to remove the plasma fraction and to place the cells in an appropriate buffer or media for subsequent processing steps. In one embodiment of the disclosure, the cells are washed with phosphate buffered saline (PBS). In an alternative embodiment, the wash solution lacks calcium and may lack magnesium or may lack many if not all divalent cations. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In one embodiment, T-cells are isolated from peripheral blood lymphocytes by lysing the red blood cells and depleting the monocytes, for example, by centrifugation through a PERCOLL™ gradient or by counterflow centrifugal elutriation. A specific subpopulation of T-cells, such as CD3+, CD28+, CD4+, CD8+, CD45RA+, and CD45RO+ T-cells, can be further isolated by positive or negative selection techniques. In certain embodiments, it may be desirable to perform the selection procedure and use the “unselected” cells in the activation and expansion process. “Unselected” cells can also be subjected to further rounds of selection.

Enrichment of a T-cell population by negative selection can be accomplished with a combination of antibodies directed to surface markers unique to the negatively selected cells. For example, to enrich for CD4+ cells by negative selection, a monoclonal antibody cocktail may include antibodies to CD14, CD20, CD1 lb, CD16, HLA-DR, and CD8. In one embodiment, a T-cell population can be selected that expresses one or more of IFN-γ, TNFα, IL-17A, IL-2, IL-3, IL-4, GM-CSF, IL-10, IL-13, granzyme B, and perform, or other appropriate molecules, e.g., other cytokines.

T-cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. In certain embodiments, cryopreserved cells are thawed and washed and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T-cells, isolated and frozen for later use in T-cell therapy for any number of diseases or conditions that would benefit from T-cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T-cells may be expanded, frozen, and used at a later time.

### Activation and Expansion of T Cells

Generally, the T-cells of the disclosure may be expanded by contact with a surface having attached thereto an agent that stimulates a CD3/TCR complex associated signal and a ligand that stimulates a costimulatory molecule on the surface of the T-cells. In particular, T-cell populations may be stimulated as described herein, such as by contact with an anti-CD3 antibody, or antigen binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4+ T-cells or CD8+ T-cells, an anti-CD3 antibody and an anti-CD28 antibody.

In certain embodiments, the primary stimulatory signal and the costimulatory signal for the T-cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the costimulatory signal is bound to a cell surface and the agent providing the primary activation signal is in solution or coupled to a surface. In certain embodiments, both agents can be in solution. In one embodiment, the agents may be in soluble form, and then cross-linked to a surface, such as a cell expressing Fc receptors or an antibody or other binding agent which will bind to the agents. In one embodiment, the two agents are immobilized on beads, either on the same bead, i.e., “cis,” or to separate beads, i.e., “trans.”

In further embodiments of the present disclosure, the cells, such as T-cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

### EXAMPLES

### Example 1. Preparation of T Cells Transduced with Modified TCRs

### Lentiviral Production

Lentivirus encoding the appropriate constructs are prepared as follows. 5×10⁶ HEK293FT-cells are seeded into a 100 mm dish and allowed to reach 70-90% confluency overnight. 2.5 μg of the indicated DNA plasmids and 20 μL Lentivirus Packaging Mix are diluted in 0.5 mL DMEM or Opti-MEM I Medium without serum and mixed gently. In a separate tube, 30 μL of transfection reagent is diluted in 0.5 mL DMEM or Opti-MEM I Medium without serum and mixed gently. The NanoFect/DMEM and DNA/DMEM solutions are mixed together and vortexed for 10-15 seconds prior to incubation of the DMEM-plasmid-reagent mixture at room temperature for 15 minutes. The complete transfection complex from the previous step is added dropwise to the plate of cells and rocked to disperse the transfection complex evenly in the plate. The plate is then incubated overnight at 37° C. in a humidified 5% CO₂ incubator. The following day, the supernatant is replaced with 10 mL fresh media and supplemented with 20 μL of ViralBoost (500×, ALSTEM). The plates are then incubated at 37° C. for an additional 24 hours. The lentivirus containing supernatant is then collected into a 50 mL sterile, capped conical centrifuge tube and put on ice. After centrifugation at 3000 rpm for 15 minutes at 4° C., the cleared supernatant is filtered with a low-protein binding 0.45 μm sterile filter and virus is subsequently isolated by ultracentrifugation at 25,000 rpm for 1.5 hours, at 4° C. The pellet is removed and re-suspended in DMEM media and Lentivirus concentrations/titers are established by quantitative RT-PCR. Any residual plasmid DNA is removed by treatment with DNase 1. The virus stock preparation is either used for infection immediately or aliquoted and stored at −80° C. for future use.

### PBMC Isolation

Peripheral Blood Mononuclear Cells (PBMCs) are prepared from whole blood. Whole blood is collected in 10 mL Heparin vacutainers and either processed immediately or stored overnight at 4° C. Approximately 10 mL of whole anti-coagulated blood is mixed with sterile phosphate buffered saline (PBS) buffer for a total volume of 20 mL in a 50 mL conical centrifuge tube. 20 mL of this blood/PBS mixture is then gently overlayed onto the surface of 15 mL of Ficoll reagent prior to centrifugation at 400×g for 30-40 min at room temperature with no brake application. The layer of cells containing PBMCs is removed carefully to minimize contamination by Ficoll. Residual Ficoll, platelets, and plasma proteins are then removed by washing the PBMCs three times with 40 mL of PBS by centrifugation at 200×g for 10 minutes at room temperature. The cells are then counted with a hemocytometer. The washed PBMCs are transferred to insulated vials and frozen at −80° C. for 24 hours before storing in liquid nitrogen for later use.

### T Cell Transduction/Transfection and Expansion

Following activation of PBMCs, cells are incubated for 24 hours at 37° C., 5% CO₂. Lentivirus is thawed on ice and 5×10⁶ lentivirus, along with 2 μL of viral transduction enhancer per mL of media is added to each well of 1×10⁶ cells. Cells are incubated for an additional 24 hours before repeating addition of virus. Alternatively, lentivirus is thawed on ice and the virus is added at 5 or 50 MOI in presence of 5 μg/mL Polybrene. Cells are spinoculated at 100×g for 100 minutes at room temperature. Cells are then grown in the continued presence of 300 IU/mL of human IL-2 for a period of 6-14 days. Cell concentrations are analyzed every 2-3 days, with media being added at that time to maintain the cell suspension at 1×10⁶ cells/mL.

### Example 2. Preparation of Soluble TCRs

Expression plasmids encoding the TCR alpha and beta chains are produced using standard molecular biology techniques. Plasmids are transformed into chemically-competent cells and grown overnight at 37° C. Protein expression is induced by the addition of Isopropyl β-D-1-thiogalactopyranoside (IPTG) to 1 mM and bacteria are grown for a further 3 hours at 37° C. Bacteria are harvested by centrifugation at 4000×g for 15 minutes and lysed in a protein extraction reagent containing DNAse. Lysis proceeds for 1 hour at room temperature with agitation before inclusion bodies are harvested by centrifugation at 10000×g for 5 minutes. Pellets are washed twice with a detergent buffer containing 1% Triton X100 and resuspended in a buffered saline solution.

Soluble TCRs are prepared by dissolving alpha and beta inclusion bodies in 6M guanidine-HCl containing 10 mM dithiothreitol and incubating at 37° C. for 30 minutes. Samples are diluted into 50 ml urea folding buffer (5 M urea; 0.4 M L-arginine; 0.1 M Tris-CI, pH 8.1; 2 mM EDTA; 6.5 mM β-mercapthoethylamine; 1.9 mM cystamine) and dialyzed against eight volumes of water overnight at 4° C., followed by dialysis for a further 24 hours in eight volumes of 10 mM Tris (8.1), with one buffer change. Dialysate (30 ml) is concentrated to 1 ml. Concentrated protein is diluted to 5 ml in phosphate-buffered saline and concentrated to 0.5 ml.

TCR fusion constructs can also be produced in mammalian cells, insect cells, or yeast cells according to known methods.

### Example 3. In Vitro Screening of a Modified TCR Produced in Examples 1 or 2 for Antigen Recognition

A modified TCR is tested for its ability to recognize antigens when separately expressed in CD8⁺ T cells and CD4⁺ T cells. PBMC from a subject is transfected as described in Zhao et al. (2006), et al., Mol. Ther. 13: 151-159 (2006) with (i) RNA encoding the WT alpha chain of the TCR and (ii) RNA encoding the WT beta chain of the TCR, or DNA encoding Green Fluorescence Protein (GFP).

Transfected cells are washed and stimulated with or without (T alone) one of the following cells: T2+ pulsed with antigen. Responder cells (1×10⁵ electroporated PBLs) and 1×10⁵ stimulator cells are incubated in a 0.2-ml culture volume in individual wells of 96-well plates. Stimulator cells and responder cells are co-cultured for 16 to 24 h. Cytokine secretion of culture supernatants diluted to the linear range of the assay is measured using commercially available ELISA kits (IFN-γ Endogen, Cambridge, Mass.). The amount of IFN-γ (pg/ml) produced by transfected CD8⁺ T cells is determined, while the amount of IFN-γ (pg/ml) produced by transfected CD4⁺ T cells is determined.

### Example 4. TCR-1 Peptide Library Biopanning

Biopanning with m13 phagemid p8 or p3 displayed peptide libraries was either performed with directly coated T cell receptor, TCR-1 (Table 1, MAGE-A3, clone IC-3) on 96-well ELISA plates or with biotin-conjugated T cell receptor immobilized on streptavidin coated paramagnetic beads. Following binding to target and washing steps, specifically bound phage were recovered by elution at pH 2.2. Enrichment of specific binding clones was generally accomplished by 3-4 rounds of successive biopanning and amplification. After 3 or 4 rounds of biopanning the resulting phage pools were infected into TG1 cells and plated out on LB-ampicillin/agar plates for clonal isolation and subsequent characterization.

### Example 5. Phagemid TCR-1 Hit Identification ELISA

For hit identification, individual colonies were grown in 96-deep well plates for 2-4 hours and infected with helper phage and induced to produce peptide displayed phagemid following an overnight growth. The next day the deep well plates were centrifuged to separate the soluble phagemid from the E. coli cells. The phagemid containing supernatants were then combined with PBS-Tween 20 (0.05%)+BSA (1%) blocking buffer and incubated in ELISA wells containing Neutravidin captured biotin-conjugated TCR-1 or Neutravidin alone (Table 1). After binding at 4 degrees the plates were washed, and specifically bound phage were detected by anti-m13 HRP conjugated antibodies using standard TMB-based chromogenic ELISA procedures. Daughter plates or individual wells were subjected to standard DNA sequencing for clonal peptide sequence identification. Table 1 below shows the quantitative ELISA results for phagemid binding to IC-3 and peptide sequence identity of the respective unique clones.

TABLE 1
Peptide Phagemid TCR-1 binding, pMHC competition ELISA data,
and peptide sequence for each of the clones.

| | | | Secondary pMHC phagemid | | | | |
|---|---|---|---|---|---|---|---|
| | Primary Phagemid | | competition ELISA | | | | |
| | binding ELISA | | IC-3 | binding with | | | |
| | | Neutravidin | binding | 200 nM | % binding | | |
| | TCR-1 | control | no pMHC | pMHC | with pMHC | SEQ | |
| clone | wells | wells | competitor | competitor | competition | ID NO: | peptide sequence |
| J043_A05 | 1.339 | 0.059 | 0.499 | 0.108 | 22% | 65 | LVWGCIWDDMCS |
| J043_A09 | 1.307 | 0.069 | 0.465 | 0.099 | 21% | 66 | WHWEPSMVWGML |
| J043_A11 | 1.314 | 0.116 | 0.743 | 0.106 | 14% | 67 | VRTWFEKFPELV |
| J044_A02 | 1.661 | 0.046 | 1.660 | 0.073 | 4% | 68 | GGNACEMTYDHTFCDP |
| J044_A05 | 0.503 | 0.076 | 0.223 | 0.070 | 31% | 69 | GGIICWFDNGLVQCSW |
| J044_A07 | 1.948 | 0.049 | 2.592 | 1.057 | 41% | 70 | GGDLCDSAWAYWYCDE |
| J044_A09 | 1.452 | 0.042 | 1.175 | 0.056 | 5% | 71 | GGESCQSVYDSSFCYD |
| J044_A10 | 1.781 | 0.297 | 1.466 | 0.195 | 13% | 72 | GGCSWIFDGLRYFSKC |
| J044_A11 | 0.931 | 0.043 | 0.750 | 0.084 | 11% | 73 | GGGMCSLVYDSVFCDQ |
| J044_A12 | 1.745 | 0.055 | 2.661 | 2.138 | 80% | 74 | GGCELYYSWSGSYDMC |
| J044_B03 | 1.955 | 0.112 | 1.615 | 0.224 | 14% | 75 | GGDCQPDSVWSYWYCR |
| J044_B05 | 1.953 | 0.158 | 1.751 | 0.279 | 16% | 76 | GGCTFVDWWVLGSPYC |
| J044_B12 | 1.303 | 0.062 | 1.452 | 0.100 | 7% | 77 | GGVACREVYDHYFCWD |
| J044_C01 | 1.989 | 0.052 | 2.799 | 0.382 | 14% | 78 | GGVSCKDVYDEAFCWT |
| J044_C02 | 1.247 | 0.053 | 0.643 | 0.058 | 9% | 79 | GGNSCSLVYDKAFCLF |
| J044_C03 | 1.951 | 0.056 | 2.497 | 0.112 | 4% | 80 | GGRRCVDVYDNAFCLI |
| J044_C04 | 1.867 | 0.047 | 1.918 | 0.107 | 6% | 81 | GGRACSDIYDAEFCGL |
| J044_C07 | 1.430 | 0.055 | 0.427 | 0.082 | 19% | 82 | GGCLMNDYYYLWGGHC |
| J044_C09 | 1.857 | 0.054 | 2.807 | 0.994 | 35% | 83 | GGTSCAQIYDFEFCYS |
| J044_E01 | 1.956 | 0.067 | 2.814 | 0.172 | 6% | 84 | GGSLCSLVYDQDFCES |
| J044_E04 | 1.433 | 0.055 | 1.193 | 0.105 | 9% | 85 | GGVPCWMVYDALFCGL |
| J044_E08 | 1.826 | 0.130 | 1.133 | 0.153 | 14% | 86 | GGGCFVSPATGFTWCV |
| J044_E09 | 1.720 | 0.054 | 0.926 | 0.058 | 6% | 87 | GGNLCHDVYDMSFCYG |
| J044_F03 | 1.118 | 0.054 | 0.426 | 0.085 | 20% | 88 | GGTFCYFDHGLVNCQW |
| J044_F06 | 1.672 | 0.058 | 1.140 | 0.066 | 6% | 89 | GGRICEEVYDWIFCES |
| J044_F07 | 2.027 | 0.067 | 2.371 | 0.832 | 35% | 90 | GGSACTRVYDYDFCYG |
| J044_G03 | 1.806 | 0.059 | 1.164 | 0.083 | 7% | 91 | GGNQCWEVYDQEFCSL |
| J044_G04 | 2.022 | 0.064 | 2.482 | 0.118 | 5% | 92 | GGERCESVYDLFFCYG |
| J044_G07 | 1.937 | 0.055 | 1.992 | 0.110 | 6% | 93 | GGVLCETVYDRDFCFA |
| J044_G09 | 1.599 | 0.055 | 0.768 | 0.083 | 11% | 94 | GGGNCSVIYDDLFCLV |
| J044_H02 | 1.771 | 0.065 | 1.706 | 0.110 | 6% | 95 | GGSACSRIYDFAFCHT |
| J044_H11 | 2.137 | 0.051 | 2.675 | 0.873 | 33% | 96 | GGHCFVSPASGEWWCV |
| J250_B08 | 1.339 | 0.220 | 1.613 | 0.266 | 17% | 97 | GGLCDLSGLWPLYC |
| J250_C02 | 3.187 | 0.097 | 1.024 | 0.697 | 68% | 98 | GGCVFYGSVDHIWYDC |
| J250_C06 | 1.151 | 0.149 | 1.701 | 0.521 | 31% | 99 | GGGYCSIVYDRLFCSS |
| J250_C07 | 2.545 | 0.082 | 1.568 | 0.412 | 26% | 100 | GGQECHTVYDVQFCSH |
| J250_D03 | 3.286 | 0.141 | 1.861 | 0.191 | 10% | 101 | GGCGFNIAAPLYGLVC |
| J250_D10 | 2.928 | 0.108 | 1.447 | 0.175 | 12% | 102 | GGQECSFIYDRVFCLV |
| J250_E07 | 1.117 | 0.153 | 2.502 | 0.468 | 19% | 103 | GGGCSYQGPWEFWYCR |
| J250_F02 | 2.839 | 0.102 | 2.365 | 0.081 | 3% | 104 | GGLHGCFDGTFVSCSW |
| J250_G02 | 3.122 | 0.152 | 3.016 | 1.965 | 65% | 105 | GGLADYCEHPMCYWYS |
| J250_H09 | 2.740 | 0.204 | 2.944 | 0.154 | 5% | 106 | GGPCVSVLQELVLEWC |
| J258_A05 | 2.437 | 0.053 | 2.839 | 0.084 | 3% | 107 | GGSTWGCIWDDMCGQA |
| J258_A07 | 2.399 | 0.057 | 3.000 | 0.079 | 3% | 108 | GGACVAENEWAYWYCR |
| J258_A09 | 1.809 | 0.142 | 3.083 | 0.113 | 4% | 109 | GGFTFTCMLHDCVYIL |
| J258_B02 | 2.509 | 0.071 | 0.232 | 0.063 | 27% | 110 | GGHHCTQVYDYSFCFM |
| J258_B05 | 2.273 | 0.057 | 1.788 | 0.050 | 3% | 111 | GGQICADTYDWVFCFE |
| J258_B06 | 2.283 | 0.056 | 3.235 | 2.931 | 91% | 112 | GGAHCHQVYDYSFCFL |
| J258_D01 | 2.572 | 0.060 | 2.020 | 0.079 | 4% | 113 | GGSFCEIVYDALFCDM |
| J258_E02 | 2.574 | 0.048 | 2.315 | 0.055 | 2% | 114 | GGLECSLTYDWEFCKY |
| J258_F04 | 2.255 | 0.183 | 2.747 | 2.164 | 79% | 115 | GGFIFSCSNDECFYFL |
| J258_F11 | 2.473 | 0.051 | 2.889 | 0.074 | 3% | 116 | GGYECRRSLDADICWI |
| J258_H06 | 2.324 | 0.381 | 1.706 | 0.070 | 4% | 117 | GGWCSVWFFDGWEWCG |
| J270_A07 | 1.865 | 0.184 | 2.403 | 0.063 | 3% | 118 | GGSFCYTHPFGYFYCR |
| J270_A10 | 3.413 | 0.097 | 2.109 | 0.063 | 3% | 119 | GGSLCGDGYGWYWMCL |
| J270_D11 | 1.605 | 0.080 | 1.931 | 1.076 | 56% | 120 | GGMWFCDWKWDSLCDV |
| J270_F09 | 1.702 | 0.065 | 0.354 | 0.059 | 17% | 121 | GGPVFCFDGTVFGCWL |
| J270_G05 | 2.456 | 0.274 | 0.356 | 0.267 | 75% | 122 | GGIRTCWHPFVVWCMS |
| J270_G10 | 2.518 | 0.072 | 1.984 | 0.068 | 3% | 123 | GGFCQALRAEYYFFCS |
| J270_H11 | 0.741 | 0.059 | 1.620 | 0.069 | 4% | 124 | GGTPFCFDGMVYACRS |
| J271_A08 | 0.998 | 0.045 | 0.395 | 0.225 | 57% | 125 | GGEWFCDWAWGVYCKA |
| J271_A11 | 0.636 | 0.045 | 1.798 | 0.056 | 3% | 126 | GGGLCWHPFVPHYYCR |
| J271_A12 | 0.534 | 0.046 | 2.382 | 0.115 | 5% | 127 | GGHECVMVYGEWEFCN |
| J271_C02 | 2.984 | 0.126 | 2.632 | 0.076 | 3% | 128 | GGSWFCDHMWLEYCGS |
| J272_A03 | 1.771 | 0.096 | 1.865 | 0.160 | 9% | 129 | GGMVCWDGPWHFVCPG |
| J272_A04 | 0.673 | 0.075 | 1.186 | 0.117 | 10% | 130 | GGGFCEDGMRWTQCIF |
| J272_A07 | 1.919 | 0.157 | 0.381 | 0.060 | 16% | 131 | GGYACWFGHGLVHCGT |
| J272_A09 | 2.705 | 0.115 | 1.523 | 0.070 | 5% | 132 | GGSEIRCDWFWCFDVL |
| J272_B04 | 2.734 | 0.196 | 3.336 | 3.472 | 104% | 133 | GGCTFVEWWHHGYKLC |
| J272_B09 | 2.427 | 0.064 | 2.370 | 0.305 | 13% | 134 | GGRVCYFDHQVVHCIW |
| J272_B10 | 1.717 | 0.068 | 1.683 | 0.075 | 4% | 105 | GGLADYCEHPMCYWYS |
| J272_D01 | 2.573 | 0.091 | 0.806 | 0.064 | 8% | 135 | GGLWCHEIYDLAFCRF |
| J272_F01 | 0.831 | 0.120 | 0.977 | 0.088 | 9% | 136 | GGCEWLPGLVHLIYHC |
| J272_G08 | 0.530 | 0.067 | 2.449 | 0.085 | 3% | 137 | GGYPCHQIYDSNFCYF |
| J272_G10 | 2.740 | 0.080 | 3.124 | 0.067 | 2% | 138 | GGCQVEWWGKPYTINC |
| J273_A03 | 1.067 | 0.068 | 3.381 | 0.118 | 3% | 139 | GGEPCHEVFDHSFCMM |
| J274_A06 | 2.619 | 0.064 | 2.460 | 0.357 | 15% | 140 | GGHCIEMIYGWMGYTC |
| J274_B01 | 2.558 | 0.066 | 3.015 | 0.757 | 25% | 141 | GGMCVDRTNWLWQIYC |
| J274_B11 | 2.545 | 0.088 | 3.315 | 1.915 | 58% | 142 | GGSCWDVYGKWAYWHC |
| J274_C08 | 1.835 | 0.073 | 3.301 | 3.165 | 96% | 143 | GGYCHPFLAWQHDYFC |
| J274_D01 | 1.033 | 0.077 | 1.190 | 0.111 | 9% | 144 | GGICREFSGEWWVWDC |
| J274_E01 | 2.635 | 0.082 | 2.114 | 0.082 | 4% | 145 | GGDPACSPSTLVCWLF |
| J274_F05 | 2.472 | 0.063 | 2.788 | 0.185 | 7% | 146 | GGYVTCHWDQSFCWFH |
| J275_B02 | 1.988 | 0.161 | 0.390 | 0.238 | 61% | 147 | GGIVYCFFDSPWCFVR |
| J275_B03 | 1.555 | 0.084 | 2.902 | 0.062 | 2% | 148 | GGVPDWCWTIGLCFGT |
| J279_A01 | 2.096 | 0.098 | 0.478 | 0.063 | 13% | 149 | GGAVYCFDGWFFTCGG |
| J279_A02 | 1.956 | 0.083 | 1.579 | 0.057 | 4% | 150 | GGQSPCFDGTVIACLG |
| J280_A05 | 2.014 | 0.179 | 1.825 | 0.057 | 3% | 151 | GGSMCEHVYDWLFCFV |
| J280_C05 | 1.008 | 0.063 | 1.954 | 0.358 | 18% | 152 | GGHFCSRVYDESFCDE |
| J280_C12 | 2.245 | 0.097 | 1.469 | 0.061 | 4% | 153 | GGVICWFQEGMVHCVS |
| J280_H09 | 2.205 | 0.065 | 1.370 | 0.047 | 3% | 154 | GGQHCNDVYDWAFCLI |
| J282_E03 | 3.012 | 0.214 | 1.870 | 0.127 | 7% | 155 | GGVLCFVHQNQTFECS |
| J282_H08 | 2.394 | 0.159 | 1.943 | 0.177 | 9% | 156 | GGGCYVHPGSGVFWCS |
| J282_H12 | 2.551 | 0.125 | 1.523 | 0.122 | 8% | 157 | GGTVHCFDGVVFSCLG |
| J287_B03 | 3.145 | 0.180 | 1.607 | 0.153 | 9% | 158 | GGHCYALTFMDYWACN |
| J288_H06 | 3.163 | 0.136 | 1.987 | 0.373 | 19% | 159 | GGMMCEHVYDFLFCLS |
| J289_B11 | 2.089 | 0.112 | 1.958 | 0.127 | 6% | 160 | GGSMIKCNFPRLCNGK |
| J289_C01 | 2.861 | 0.100 | 1.901 | 0.208 | 11% | 161 | GGFCEQGQFMAAWHRC |
| J289_D01 | 1.445 | 0.096 | 1.838 | 0.092 | 5% | 162 | GGDLLQCPQAVSCRPR |
| J289_D10 | 1.867 | 0.120 | 1.881 | 0.132 | 7% | 163 | GGDVRCFMRMMECVLL |

### Example 6. TCR-1 Phagemid Competition ELISA Assay

Phagemid peptide clones that specifically bound TCR-1 were next tested to determine whether they bound within the antigen binding space of the antibody cell receptor, by target-based competition assay. TCR-1 immobilized and blocked 96-well ELISA plates similar to above were prepared. Human MAGE-A3 pMHC was added to wells to block the active binding site. After a brief incubation period phagemid supernatants were added to both sets of wells. Following further incubation at 4 degrees the plates were washed and specifically bound phage were detected by anti-m13 HRP conjugated antibodies using standard TMB-based chromogenic ELISA procedures. Phagemid clones binding within the antigenic binding pocket would be blocked and be identified by a decreased ELISA signal, compared to a well lacking previous pMHC blockade.

Table 1 above shows the ELISA results for the pMHC competition of phagemid binding to TCR-1, along with primary ELISA results and sequence identities of the respectively tested phagemid clones.

### Example 7. Bacterial Expression of Reformatted T Cell Receptor (TCR) Peptide Fusions

The following procedure was used to reformat peptides found above into recombinant TCR fusions. T cell receptors are comprised of an alpha chain complexed with a beta chain. Each alpha and beta chains include the entire extracellular domain and lack the membrane spanning and intracellular domains. Additional beta chain constructs with TCR-1 binding peptides fused by flexible or proteolytically labile linkers were similarly synthesized. Each of the individual T cell receptor chains were overexpressed in E. coli and recovered from inclusion bodies. Specifically, genes encoding the alpha or beta subunits with or without additional peptide or protein fusions added to either the amino or carboxy-termini were synthesized using E. coli codon optimization. Additionally, the C-terminus of the alpha subunit has appended a poly histidine epitope for protein purification purposes and to the C-terminus of the beta subunit a BirA biotinylation substrate (“Avitag”) was appended for enzymatic site specific biotin conjugation. Following protein expression, inclusion bodies were isolated and then dissolved in solubilization buffer (8 M urea, 25 mM MES pH 6.0, 10 mM EDTA, 0.1 mM DTT), while TCRs were dissolved in the solubilization buffer containing 6 M guanidine hydrochloride (GnHCl). Thirty milligrams each of TCR alpha and TCR beta were diluted into 500 mL refolding buffer [3 M urea, 0.2 M Arg-HCl, 150 mM Tris-HCl pH 8.0, 1.5 mM reduced glutathione, 0.15 mM oxidized glutathione and stirred at 4° C. for 72 h. Refolded TCR was dialyzed at 4° C. for 24 h in 4 L dialysis buffer (10 mM Tris pH 8.5, 50 mM NaCl) and then for an additional 24 h in fresh 4 L dialysis buffer. The resultant TCR complexes are concentrated and purified using Ni-NTA, and size-exclusion chromatography.

Table 2 below exemplifies the sequences of the recombinant TCRs and fragments thereof.

TABLE 2
Sequences.

| TCR ID | Alpha | Beta |
|---|---|---|
| TCR-1 | IC-3 parental alpha chain | IC-3 parental beta chain |
| | (Seq ID NO: 46) | (Seq ID NO: 47) |
| TCR-2 | IC-3 parental alpha chain | IC-3 beta subunit + |
| | (Seq ID NO: 46) | MAGE-A3 peptide connected |
| | | to N-term via 26 amino acid |
| | | noncleavable, flexible linker |
| | | (Seq ID NO: 48) |
| TCR-3 | IC-3 parental alpha chain | IC-3 beta subunit + |
| | (Seq ID NO:46) | MAGE-A3 peptide connected |
| | | to N-term via 26 amino acid |
| | | cleavable, flexible linker |
| | | (Seq ID NO: 49) |
| TCR-4 | IC-3 parental alpha chain | IC-3 beta subunit + |
| | (Seq ID NO:46) | Peptide-5 connected to |
| | | N-term via 18 amino acid |
| | | cleavable, flexible linker |
| | | (Seq ID NO: 50) |
| TCR-5 | IC-3 parental alpha chain | IC-3 beta subunit + |
| | (Seq ID NO: 46) | Peptide-5 connected to |
| | | N-term via 26 amino acid |
| | | cleavable, flexible linker |
| | | (Seq ID NO: 51) |
| TCR-6 | IC-3 alpha subunit + Peptide-5 | IC-3 parental beta chain |
| | connected to N-term via 18 amino | (Seq ID NO: 47) |
| | acid cleavable, flexible linker | |
| | (Seq ID NO: 52) | |
| TCR-7 | IC-3 alpha subunit + Peptide-5 | IC-3 parental beta chain |
| | connected to N-term via 26 amino | (Seq ID NO: 47) |
| | acid cleavable, flexible linker | |
| | (Seq ID NO: 53) | |
| TCR-8 | MAGE-A3 alpha chain variant #8 | MAGE-A3 beta chain variant #7 |
| | (Seq ID NO: 54) | (Seq ID NO: 55) |
| TCR-9 | MAGE-A3 alpha chain variant #6 | MAGE-A3 beta chain variant #10 |
| | (Seq ID NO: 56) | (Seq ID NO: 57) |
| TCR-10 | MAGE-A3 alpha chain variant #6 | MAGE-A3 beta chain variant #11 |
| | (Seq ID NO: 56) | (Seq ID NO: 58) |

IC-3 parental alpha chain
(Seq ID NO: 46)
MQEVTQIPAALSVPEGENLVLNCSFTDSAIYNLQWFRQDPGKGLTSLLYV
RPYQREQTSGRLNASLDKSSGRSTLYIAASQPGDSATYLCAVRPGGAGPF
FVVFGKGTKLSVIPNIQNPDPAVYQLRDSKSSDKSVCLFTDFDSQTNVSQ
SKDSDVYITDKCVLDMRSMDFKSNSAVAWSNKSDFACANAFNNSIIPEDT
FFPSPESSggHHHHHHHH
IC-3 parental beta chain
(Seq ID NO: 47)
MKAGVTQTPRYLIKTRGQQVTLSCSPISGHRSVSWYQQTPGQGLQFLFEY
FSETQRNKGNFPGRFSGRQFSNSRSEMNVSTLELGDSALYLCASSFNMAT
GQYFGPGTRLTVTEDLKNVFPPEVAVFEPSEAEISHTQKATLVCLATGFY
PDHVELSWWVNGKEVHSGVCTDPQPLKEQPALNDSRYALSSRLRVSATFW
QNPRNHFRCQVQFYGLSENDEWTQDRAKPVTQIVSAEAWGRADggGLNDI
FEAQKIEWHE
IC-3 beta subunit + MAGE-A3 peptide connected to
N-term via 26 amino acid noncleavable, flexible
linker
(Seq ID NO: 48)
MEVDPIGHLYGSSGGSGGSGGSGGGSGGGSGGSSGTKAGVTQTPRYLIKT
RGQQVTLSCSPISGHRSVSWYQQTPGQGLQFLFEYFSETQRNKGNFPGRF
SGRQFSNSRSEMNVSTLELGDSALYLCASSFNMATGQYFGPGTRLTVTED
LKNVFPPEVAVFEPSEAEISHTQKATLVCLATGFYPDHVELSWWVNGKEV
HSGVCTDPQPLKEQPALNDSRYALSSRLRVSATFWQNPRNHFRCQVQFYG
LSENDEWTQDRAKPVTQIVSAEAWGRADggGLNDIFEAQKIEWHE
IC-3 beta subunit + MAGE-A3 peptide connected to
N-term via 26 amino acid cleavable, flexible
linker
(Seq ID NO: 49)
MEVDPIGHLYGGGGSSGGSGGSGLSGRSDNHGSSGTKAGVTQTPRYLIKT
RGQQVTLSCSPISGHRSVSWYQQTPGQGLQFLFEYFSETQRNKGNFPGRF
SGRQFSNSRSEMNVSTLELGDSALYLCASSFNMATGQYFGPGTRLTVTED
LKNVFPPEVAVFEPSEAEISHTQKATLVCLATGFYPDHVELSWWVNGKEV
HSGVCTDPQPLKEQPALNDSRYALSSRLRVSATFWQNPRNHFRCQVQFYG
LSENDEWTQDRAKPVTQIVSAEAWGRADggGLNDIFEAQKIEWHE
IC-3 beta subunit + Peptide-5 connected to N-term
via 18 amino acid cleavable linker
(Seq ID NO: 50)
MGGVSCKDVYDEAFCWTGGGGSLSGRSDNHGSSGTKAGVTQTPRYLIKTR
GQQVTLSCSPISGHRSVSWYQQTPGQGLQFLFEYFSETQRNKGNFPGRFS
GRQFSNSRSEMNVSTLELGDSALYLCASSFNMATGQYFGPGTRLTVTEDL
KNVFPPEVAVFEPSEAEISHTQKATLVCLATGFYPDHVELSWWVNGKEVH
SGVCTDPQPLKEQPALNDSRYALSSRLRVSATFWQNPRNHFRCQVQFYGL
SENDEWTQDRAKPVTQIVSAEAWGRADggGLNDIFEAQKIEWHE
IC-3 beta subunit + Peptide-5 connected to N-term
via 26 amino acid cleavable, flexible linker
(Seq ID NO: 51)
MGGVSCKDVYDEAFCWTGGGGSSGGSGGSGLSGRSDNHGSSGTKAGVTQT
PRYLIKTRGQQVTLSCSPISGHRSVSWYQQTPGQGLQFLFEYFSETQRNK
GNFPGRFSGRQFSNSRSEMNVSTLELGDSALYLCASSFNMATGQYFGPGT
RLTVTEDLKNVFPPEVAVFEPSEAEISHTQKATLVCLATGFYPDHVELSW
WVNGKEVHSGVCTDPQPLKEQPALNDSRYALSSRLRVSATFWQNPRNHFR
CQVQFYGLSENDEWTQDRAKPVTQIVSAEAWGRADggGLNDIFEAQKIEW
HE
IC-3 alpha subunit + Peptide-5 connected to N-term
via 18 amino acid cleavable, flexible linker
(Seq ID NO: 52)
MGGVSCKDVYDEAFCWTGGGGSLSGRSDNHGSSGTKQEVTQIPAALSVPE
GENLVLNCSFTDSAIYNLQWFRQDPGKGLTSLLYVRPYQREQTSGRLNAS
LDKSSGRSTLYIAASQPGDSATYLCAVRPGGAGPFFVVFGKGTKLSVIPN
IQNPDPAVYQLRDSKSSDKSVCLFTDFDSQTNVSQSKDSDVYITDKCVLD
MRSMDFKSNSAVAWSNKSDFACANAFNNSIIPEDTFFPSPESSggHHHHH
HHH
IC-3 alpha subunit + Peptide-5 connected to N-term
via 26 amino acid cleavable, flexible linker
(Seq ID NO: 53)
MGGVSCKDVYDEAFCWTGGGGSSGGSGGSGLSGRSDNHGSSGTKQEVTQI
PAALSVPEGENLVLNCSFTDSAIYNLQWFRQDPGKGLTSLLYVRPYQREQ
TSGRLNASLDKSSGRSTLYIAASQPGDSATYLCAVRPGGAGPFFVVFGKG
TKLSVIPNIQNPDPAVYQLRDSKSSDKSVCLFTDFDSQTNVSQSKDSDVY
ITDKCVLDMRSMDFKSNSAVAWSNKSDFACANAFNNSIIPEDTFFPSPES
SggHHHHHHHH
MAGE-A3 alpha chain variant #8
(Seq ID NO: 54)
MQEVTQIPAALSVPEGENLVLNCSFTDSAIYNLQWFRQDPGKGLTSLLLV
RPYQREQTSGRLNASLDKSSGRSTLYIAASQPGDSATYLCAVRPGGAGSY
QLTFGKGTKLSVIPNIQNPDPAVYQLRDSKSSDKSVCLFTDFDSQTNVSQ
SKDSDVYITDKCVLDMRSMDFKSNSAVAWSNKSDFACANAFNNSIIPEDT
FFPSPESSggHHHHHHHH
MAGE-A3 beta variant #6
(Seq ID NO: 55)
MKAGVTQTPRYLIKTRGQQVTLSCSPISGHRSVSWYQQTPGQGLQFLFEY
FSETQRNKGNFPGRFSGRQFSNSRSEMNVSTLELGDSALYLCASSPNMAD
EQYFGPGTRLTVTEDLKNVFPPEVAVFEPSEAEISHTQKATLVCLATGFY
PDHVELSWWVNGKEVHSGVCTDPQPLKEQPALNDSRYALSSRLRVSATFW
QDPRNHFRCQVQFYGLSENDEWTQDRAKPVTQIVSAEAWGRADggGLNDI
FEAQKIEWHE
MAGE-A3 alpha chain variant #6
(Seq ID NO: 56)
MQEVTQIPAALSVPEGENLVLNCSFTDSAIYNLQWFRQDPGKGLTSLLLI
QSSQREQTSGRLNASLDKSSGRSTLYIAASQPGDSATYLCAVRPGGAGSY
QLTFGKGTKLSVIPNIQNPDPAVYQLRDSKSSDKSVCLFTDFDSQTNVSQ
SKDSDVYITDKCVLDMRSMDFKSNSAVAWSNKSDFACANAFNNSIIPEDT
FFPSPESSggHHHHHHHH
MAGE-A3 beta variant #10
(Seq ID NO: 57)
KAGVTQTPRYLIKTRGQQVTLSCSPISGHRSVSWYQQTPGQGLQFLFEYT
DMTLRNKGNFPGRFSGRQFSNSRSEMNVSTLELGDSALYLCASSPNMADE
QYFGPGTRLTVTEDLKNVFPPEVAVFEPSEAEISHTQKATLVCLATGFYP
DHVELSWWVNGKEVHSGVCTDPQPLKEQPALNDSRYALSSRLRVSATFWQ
DPRNHFRCQVQFYGLSENDEWTQDRAKPVTQIVSAEAWGRADggGLNDIF
EAQKIEWHE
MAGE-A3 beta variant #11
(Seq ID NO: 58)
KAGVTQTPRYLIKTRGQQVTLSCSPISGHRSVSWYQQTPGQGLQFLFEYF
DMLLRNKGNFPGRFSGRQFSNSRSEMNVSTLELGDSALYLCASSPNMADE
QYFGPGTRLTVTEDLKNVFPPEVAVFEPSEAEISHTQKATLVCLATGFYP
DHVELSWWVNGKEVHSGVCTDPQPLKEQPALNDSRYALSSRLRVSATFWQ
DPRNHFRCQVQFYGLSENDEWTQDRAKPVTQIVSAEAWGRADggGLNDIF
EAQKIEWHE

### Example 8. TCR Functional Binding

Prior to panning against a T cell receptor (TCR), the TCR protein was qualified for functional binding to its cognate pMHC. TCR-1 was qualified by its ability to bind to cognate MAGE-A3 pMHC using a ForteBio Red96 Octet instrument that utilizes bio-layer interferometry (BLI) to measure binding kinetics in real time. FIG. 4 is an exemplary BLI sensorgram and affinity of TCR-1 binding to MAGE-A3 pMHC in realtime.

BLI based kinetic binding of TCR-1 to cognate MAGE-A3 pMHC was measured using a ForteBio OctetRED96 instrument. Biotinylated peptides were first captured on streptavidin biosensors. Sensors were quenched using excess biocytin and then baselined in buffer. TCR-1 was titrated in a 2-fold dilution series starting from 100 nM and was associated onto the pMHC loaded biosensor. Association signal was monitored in real-time. Biosensors were then transferred to buffer and the dissociation of TCR was measured in real-time. Response data was baseline corrected and fit to a 1:1 binding model used to calculate association and dissociation rates.

Peptides identified from panning efforts were classified as hits based on clonal phagemid ELISA data. Of the peptide hits discovered and sequenced from panning efforts, Table 3 below lists those peptides selected for synthesis. Peptides were synthesized with a biotinylated linker and C-terminal amidation. Post synthesis peptides were characterized for their binding to the relevant target TCR, TCR-1.

Data in Table 3 includes kinetic binding and equilibrium binding results as well as inhibition characteristics. Several peptides were made as negative controls, including Peptide-34 and Peptide-35. Peptide-40 is another negative control peptide of the following sequence: GGHCVDMVDFYQQTCQGGGGS[PEG4]Lys(biotin)-NH₂ (SEQ ID NO: 164). Cysteines that form an intramolecular disulfide are highlighted. Peptides with such cysteines are cyclic peptides.

### Example 9. Peptide Characterization

Post synthesis, peptides were characterized first by their ability to bind the relevant TCR target protein. For example, peptides identified from phage panning against the TCR-1, were subsequently synthesized and screened for their ability to bind TCR-1 in both kinetic binding and equilibrium binding experiments. Kinetic binding experiments were performed on a ForteBio 96Red Octet instrument that utilizes bio-layer interferometry while equilibrium binding was evaluated using standard enzyme linked immunosorbent assays (ELISAs). Synthetic peptides that clearly bind the target TCR-1, by both kinetic and equilibrium binding, were further evaluated for their ability to inhibit TCR-1 binding to its cognate MAGE-A3 pMHC. Dose dependent inhibition of TCR-1 binding to MAGE-A3 pMHC was conducted using a fixed concentration of TCR-1 incubated with a dilution series of synthetic peptides followed by association of the mixtures to immobilized biotinylated MAGE-A3 pMHC. Biotinylated MAGE-A3 pMHC was immobilized using streptavidin biosensors kinetic binding experiments or captured on neutravidin coated plates for ELISAs.

Kinetic binding: BLI based kinetic binding of TCR-1 to synthetic peptides or biotinylated pMHC was measured using a ForteBio Octet RED96 instrument. Biotinylated peptides or pMHC were first captured on streptavidin biosensors. Sensors were quenched using excess biocytin and then baselined in buffer. TCR-1 was titrated in a 2-fold dilution series starting from 100 nM and was associated onto the loaded biosensor. Association signal was monitored in real-time. Biosensors were then transferred to buffer and the dissociation of TCR was measured in real-time.

ELISA-based binding: High binding plates were first coated with neutravidin. Neutravidin coated plates were blocked using bovine serum albumin in buffer and washed. Biotinylated peptide or pMHC at a single concentration of 100 nM was captured on neutravidin coated plates, quenched using excess biocytin, and washed. TCR-1 was prepared in a half-log dilution series starting from 10 uM. TCR was then titrated onto the peptide or pMHC captured plates for 1 hour and washed. A secondary horse radish peroxidase antibody conjugate that recognizes the histag present on the TCR-1 was then added to the plate at 1 μg/mL for 1 hour and washed. Plates were then developed using tetramethylbenzidine (TMB) for 5-10 min and stopped using acid.

Peptide inhibition studies using BLI measurements in real time: Inhibition of kinetic binding of TCR-1 to MAGE-A3 pMHC was measured using an ForteBio Octet RED96 instrument. Inhibitory peptide titrated in a 2-fold dilution series starting from 100 uM was first incubated with a constant concentration of 50 nM TCR-1. Zero concentration of inhibitory peptide (100% binding) or zero concentration of TCR (0% binding) was used as a control. Biotinylated pMHC was captured on streptavidin biosensors. Sensors were quenched using excess biocytin and then baselined in buffer. Inhibitory peptide and TCR mixtures were associated onto the pMHC loaded biosensor. Association signal was monitored in real-time. Biosensors were then transferred to buffer and the dissociation signal was measured in real-time.

Peptide inhibition in competitive binding ELISAs: High binding plates were first coated with neutravidin. Neutravidin coated plates were blocked using bovine serum albumin in buffer and washed. Biotinylated pMHC at a single concentration of 100 nM was captured on neutravidin coated plates, quenched using excess biocytin, and washed. Inhibitory peptide was titrated in a half-log dilution series starting from 100 uM and incubated with a constant concentration of 1 nM TCR-1. Inhibitory peptide and TCR mixtures were then added to the pMHC captured plates for 30 min and washed. A secondary horse radish peroxidase antibody conjugate that recognizes the histag present on the TCR-1 was then added to the plate at 1 μg/mL for 30 min and washed. Plates were then developed using tetramethylbenzidine (TMB) for 5-10 min and stopped using acid.

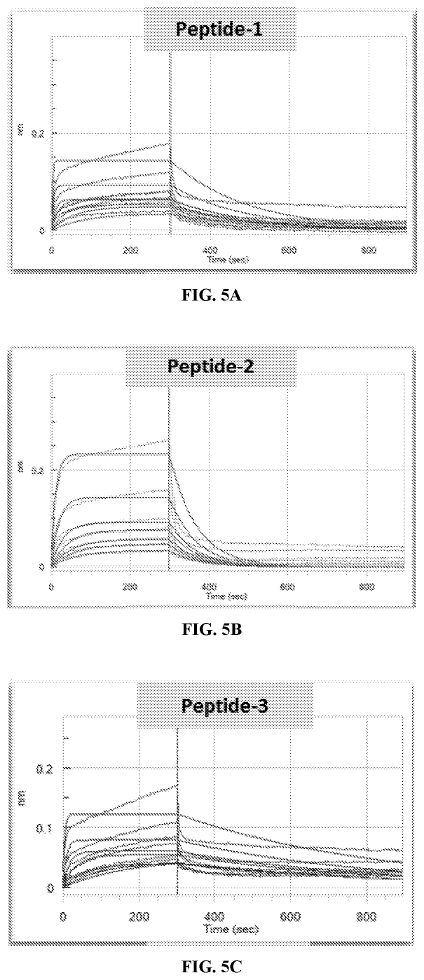

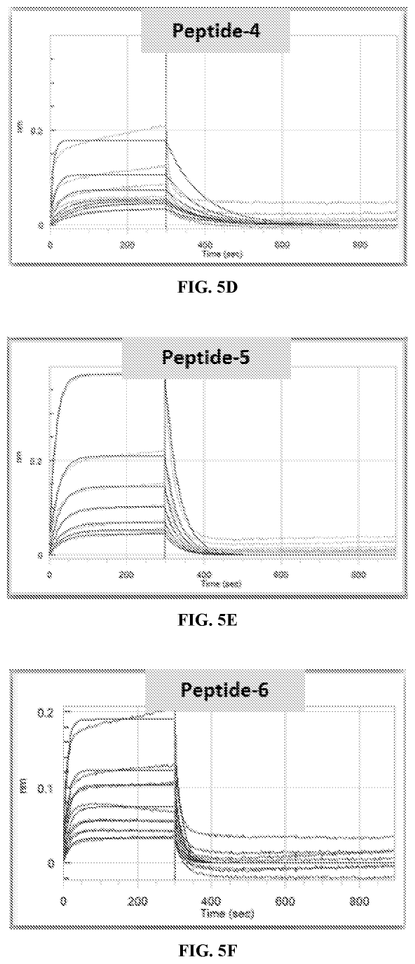

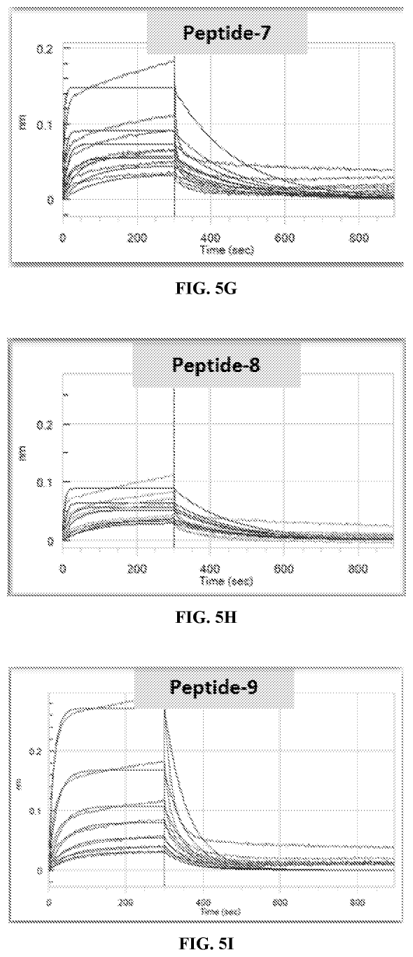

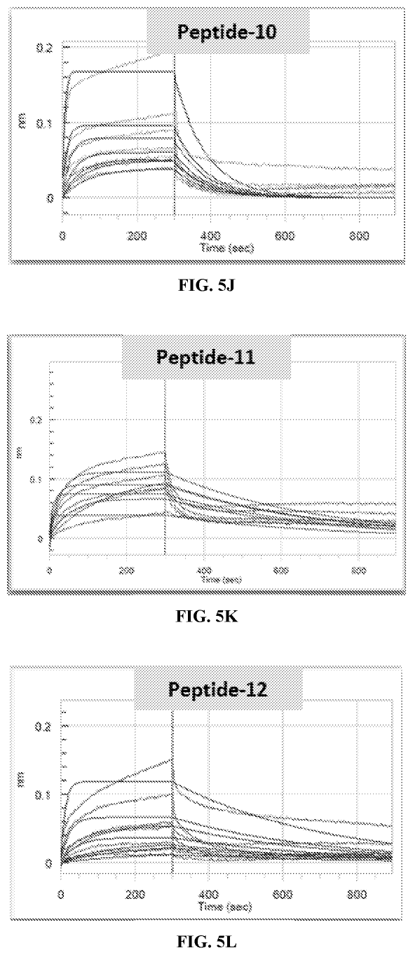

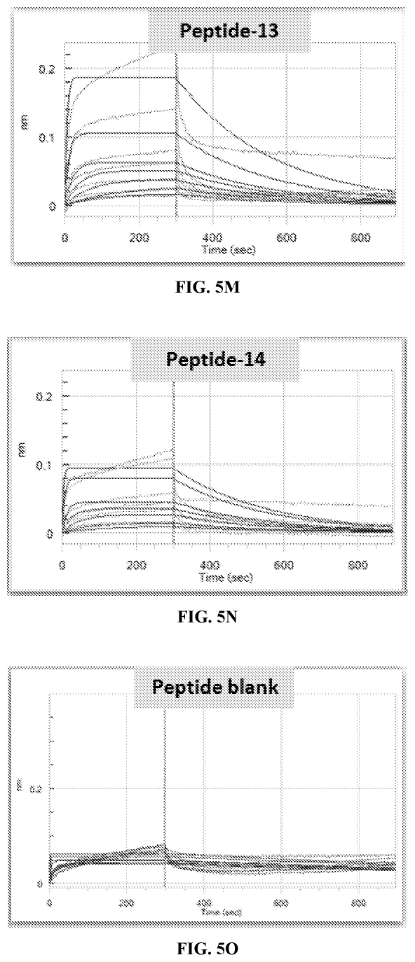

Synthetic peptides were first characterized for their ability to bind TCR-1. TCR-1 binding to synthetic peptides was examined initially via kinetic binding on the ForteBio Octet instrument. FIG. 5A-FIG. 5O are exemplary kinetic binding sensorgrams for TCR-1 binding to synthetic peptides. BLI based kinetic binding of TCR-1 to peptides was measured using a ForteBio Octet RED96 instrument. Biotinylated peptides were first captured on streptavidin biosensors. Sensors were quenched using excess biocytin and then baselined in buffer. TCR-1 was titrated in a 2-fold dilution series starting from 100 nM and was associated onto the peptide loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. Data is reported in Table 3.

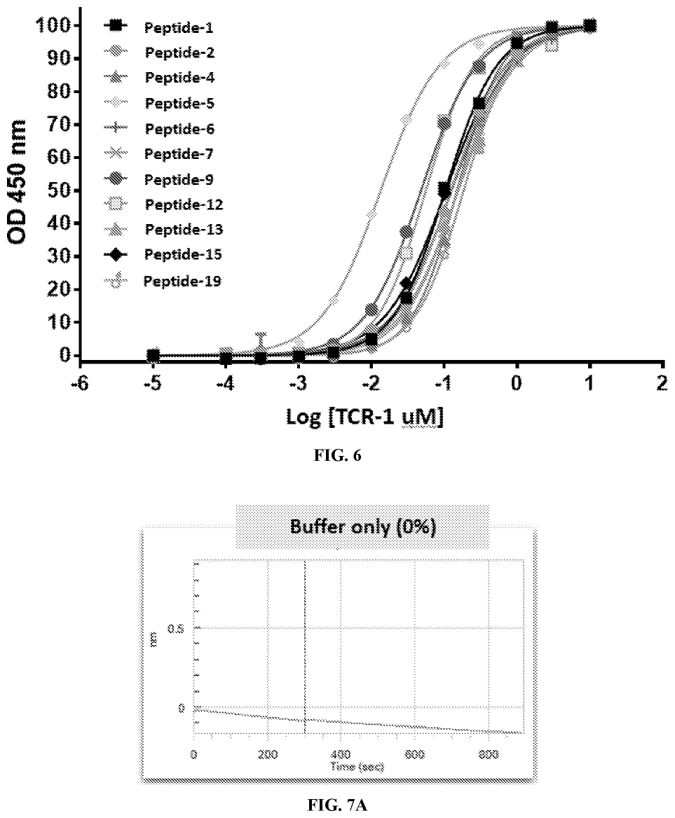

Next, TCR-1 binding to peptides was examined in a standard ELISA format. The histag present on the TCR-1 enables the use of an anti-histag secondary HRP conjugate antibody for detection of bound TCR-1 in the ELISA. High binding plates were first coated with neutravidin. Neutravidin coated plates were blocked using bovine serum albumin in buffer and washed. Biotinylated peptide at a single concentration of 100 nM was captured on neutravidin coated plates, quenched using excess biocytin, and washed. TCR-1 was prepared in a half-log dilution series starting from 10 uM. TCR was then titrated onto the peptide captured plates for 1 hour and washed. A secondary horse radish peroxidase antibody conjugate that recognizes the histag present on the TCR-1 was then added to the plate at 1 ug/mL for 1 hour and washed. Plates were then developed using tetramethylbenzidine (TMB) for 5-10 min and stopped using acid. Absorbance at 450 nm was measured for each plate and plotted versus log-scale TCR concentration. Concentration of TCR-1 that exhibits half the maximum saturation signal was calculated using Graphpad Prism 6.0 and reported as an EC50. Data for all peptides tested are in Table 3. FIG. 6 exemplifies binding of TCR-1 to peptides by ELISA. Peptide-5 exhibits the strongest binding to TCR-1 in this data set.

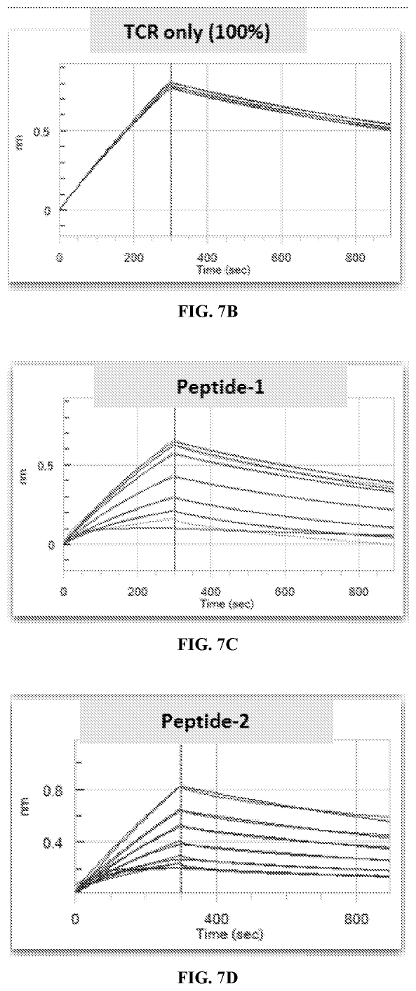

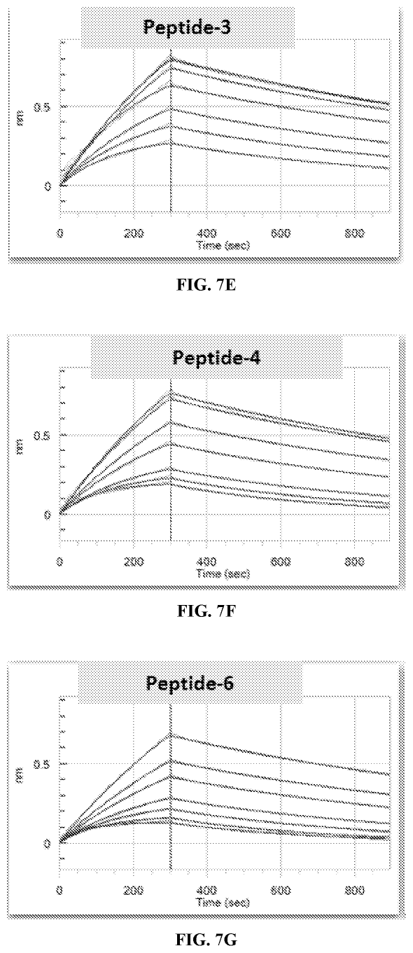

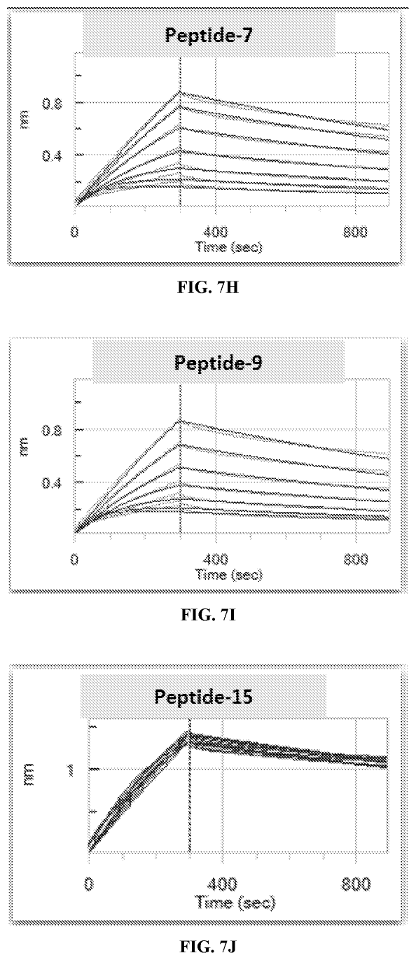

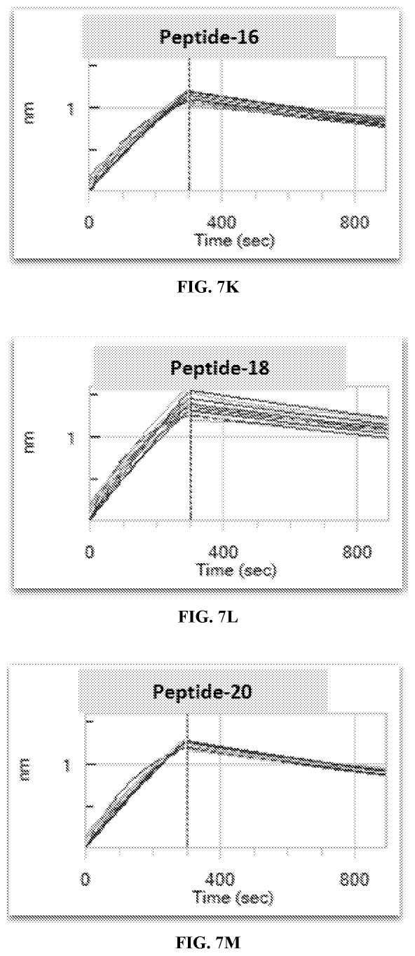

Synthetic peptides that bind to TCR-1 were further evaluated for their ability inhibit TCR-1 recognition of its cognate MAGE-A3 pMHC. Peptides that inhibit the binding of TCR-1 to MAGE-A3 pMHC are functional inhibitory peptides. Synthetic peptide binders were evaluated in both kinetic binding mode and ELISA formats for their ability to inhibit TCR-1 binding of MAGE-A3 pMHC. FIG. 7A-FIG. 7M exemplifies peptide inhibition of TCR-1 kinetic binding to MAGE-A3 pMHC. Example data sets that highlight dose dependent inhibition of TCR-1 binding to MAGE-A3 pMHC using peptides measured using BLI Octet instrument.

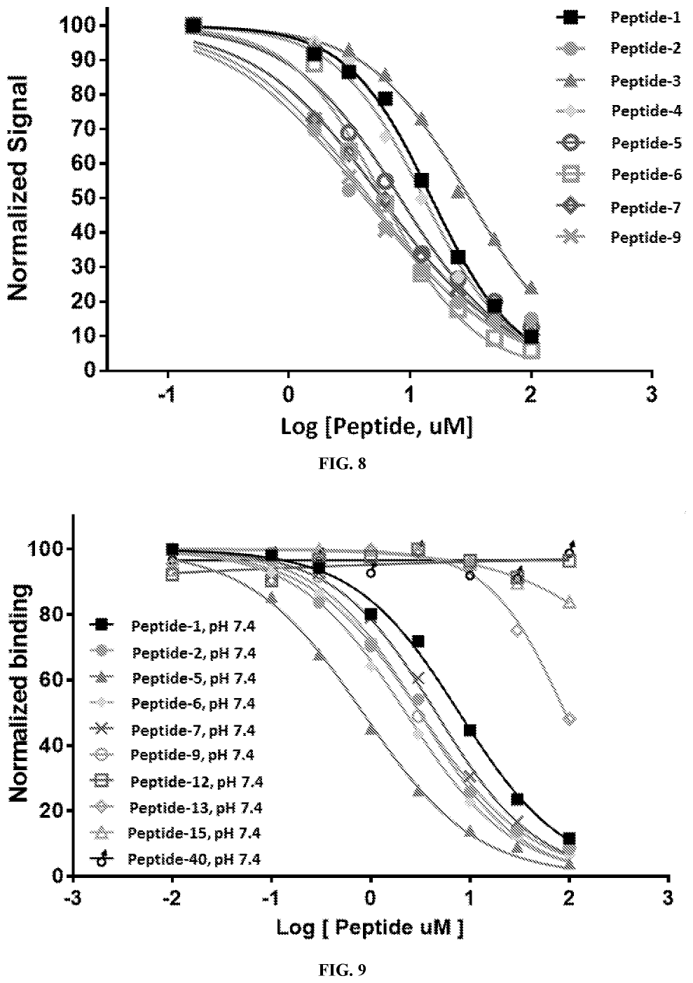

Inhibition of kinetic binding of TCR-1 to MAGE-A3 pMHC was measured using an ForteBio Octet RED96 instrument. Inhibitory peptide titrated in a 2-fold dilution series starting from 100 uM was first incubated with a constant concentration of 50 nM TCR-1. Zero concentration of inhibitory peptide (100% binding) or zero concentration of TCR (0% binding) was used as a control. Biotinylated pMHC was captured on streptavidin biosensors. Sensors were quenched using excess biocytin and then baselined in buffer. Inhibitory peptide and TCR-1 mixtures were associated onto the pMHC loaded biosensor. Association signal was monitored in real-time. Biosensors were then transferred to buffer and the dissociation signal was measured in real-time. Data was baseline corrected. The maximal association signal was normalized from 100% (0 uM inhibitory peptide control) to 0% (0 uM TCR control) and plotted versus log-scale inhibitory peptide concentration. Graphpad Prism 6.0 was used to calculate the inhibitory concentration of peptide required to achieve 50% maximal signal (IC50). The IC50s for inhibitory peptides are listed in Table 3. Several peptides were identified to be functionally active inhibitors while others were not. FIG. 8 exemplifies dose dependent inhibition of TCR-1 binding to MAGE-A3 pMHC using peptides measured using BLI Octet instrument.

High binding plates were first coated with neutravidin. Neutravidin coated plates were blocked using bovine serum albumin in buffer and washed. Biotinylated pMHC at a single concentration of 100 nM was captured on neutravidin coated plates, quenched using excess biocytin, and washed. Inhibitory peptide was titrated in a half-log dilution series starting from 100 uM and incubated with a constant concentration of 1 nM TCR-1. Inhibitory peptide and TCR mixtures were then added to the pMHC captured plates for 30 min and washed. A secondary horse radish peroxidase antibody conjugate that recognizes the histag present on the TCR-1 was then added to the plate at 1 ug/mL for 30 min and washed. Plates were then developed using tetramethylbenzidine (TMB) for 5-10 min and stopped using acid. Absorbance at 450 nm was measured for each plate and plotted versus log-scale inhibitory peptide concentration. IC50 was calculated as the concentration of peptide that inhibits 50% maximal binding signal. IC50 values for the peptides tested are listed in Table 3. FIG. 9 exemplifies dose dependent inhibition of TCR-1 binding to MAGE-A3 pMHC using peptides measured in competitive binding ELISA.

Based on the binding and inhibition data collected for synthetic peptides, Peptide-5 was of highest interest. Peptide-5 had the lowest EC50, IC50, and affinity (KD) values measured against the TCR target, TCR-1. Peptide-5 was therefore selected as the lead inhibitory peptide. Peptide-5 was evaluated in several additional binding experiments, including binding at acidic pH and binding to other TCRs closely related to TCR-1. Peptide-5 was able to bind TCR-1 at all pHs tested with equal affinity. Peptide-5 binding was selective for TCR-1.

BLI based kinetic binding of TCR-1 to inhibitory peptides was measured using a ForteBio Octet RED96 instrument. Biotinylated peptides were first captured on streptavidin biosensors. Sensors were quenched using excess biocytin and then baselined in buffer. TCR-1 was titrated in a 2-fold dilution series starting from 100 nM and was associated onto the inhibitory peptide loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. FIG. 10 is an exemplary BLI sensorgram and affinity of TCR-1 binding to Peptide-5 in realtime.

High binding plates were first coated with neutravidin. Neutravidin coated plates were blocked using bovine serum albumin in buffer and washed. Biotinylated inhibitory peptide or pMHC control at a single concentration of 100 nM was captured on neutravidin coated plates, quenched using excess biocytin, and washed. TCR-1 was prepared in a half-log dilution series starting from 10 uM. TCR-1 was then titrated onto the inhibitory peptide or pMHC captured plates for 1 hour and washed. A secondary horse radish peroxidase antibody conjugate that recognizes the TCR was then added to the plate at 1 ug/mL for 1 hour and washed. Plates were then developed using tetramethylbenzidine (TMB) for 5-10 min and stopped using acid. Absorbance at 450 nm was measured for each plate and plotted versus log-scale TCR concentration. The concentration of TCR-1 that exhibits 50% maximum saturation signal was calculated in Graphpad Prism 6.0 and shown in the summary table as EC50. FIG. 11 exemplifies TCR-1 binding of MAGE-A3 pMHC or Peptide-5 by ELISA.

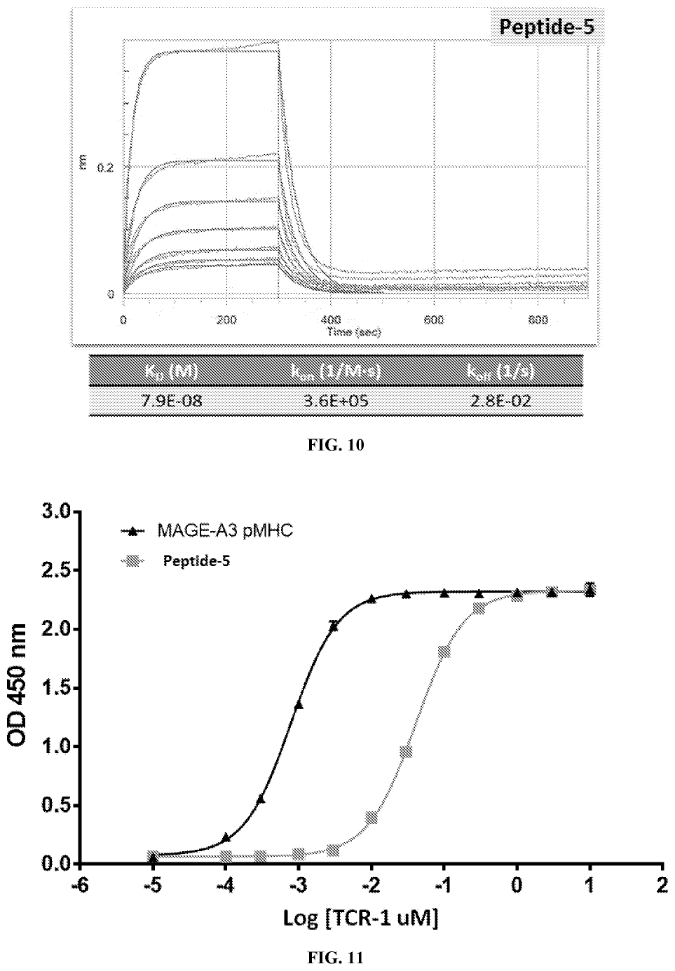

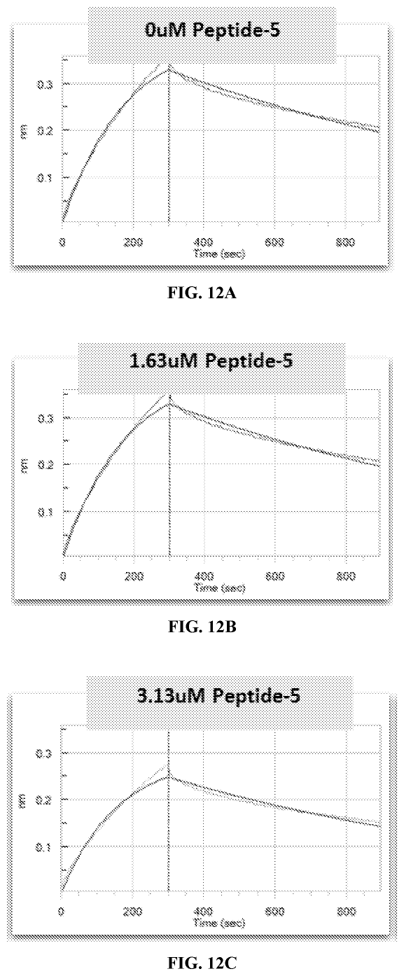

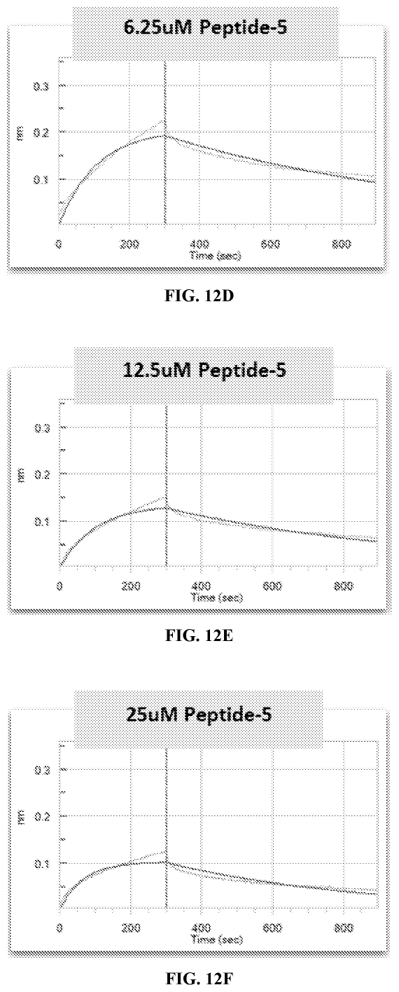

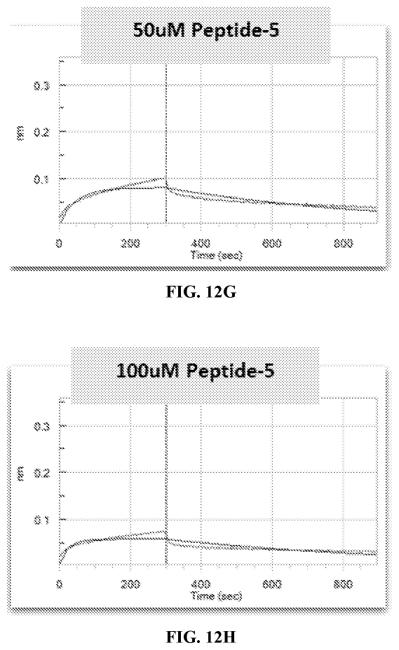

Inhibition of kinetic binding of TCR-1 to MAGE-A3 pMHC was measured using an ForteBio Octet RED96 instrument. Inhibitory peptide titrated in a 2-fold dilution series starting from 100 uM was first incubated with a constant concentration of 50 nM TCR. Zero concentration of inhibitory peptide (100% binding) or zero concentration of TCR (0% binding) was used as a control. Biotinylated pMHC was captured on streptavidin biosensors. Sensors were quenched using excess biocytin and then baselined in buffer. Inhibitory peptide and TCR mixtures were associated onto the pMHC loaded biosensor. Association signal was monitored in real-time. Biosensors were then transferred to buffer and the dissociation signal was measured in real-time. Data was baseline corrected. The maximal association signal was normalized from 100% (0 nM inhibitory peptide control) to 0% (0 nM TCR control) and plotted versus log-scale inhibitory peptide concentration. Graphpad Prism 6.0 was used to calculate the inhibitory concentration of peptide required to achieve 50% maximal signal (IC50). FIG. 12-FIG. 12H are exemplary sensorgrams for Peptide-5 dose dependent inhibition of kinetic binding of TCR-1 to cognate MAGE-A3 pMHC.

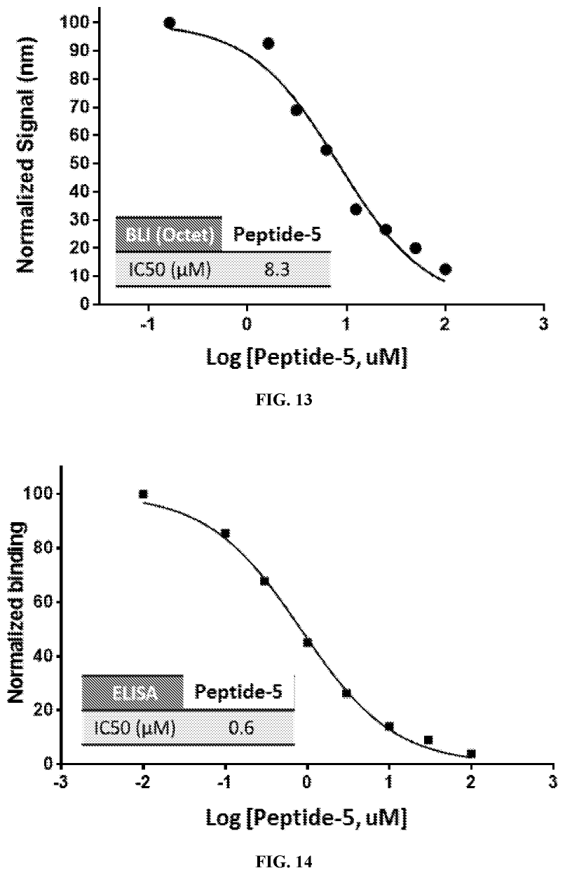

Inhibition of kinetic binding of TCR-1 to MAGE-A3 pMHC was measured using an ForteBio Octet RED96 instrument. Inhibitory peptide titrated in a 2-fold dilution series starting from 100 uM was first incubated with a constant concentration of 50 nM TCR. Zero concentration of inhibitory peptide (100% binding) or zero concentration of TCR (0% binding) was used as a control. Biotinylated pMHC was captured on streptavidin biosensors. Sensors were quenched using excess biocytin and then baselined in buffer. Inhibitory peptide and TCR mixtures were associated onto the pMHC loaded biosensor. Association signal was monitored in real-time. Biosensors were then transferred to buffer and the dissociation signal was measured in real-time. Data was baseline corrected. The maximal association signal was normalized from 100% (0 nM inhibitory peptide control) to 0% (0 nM TCR control) and plotted versus log-scale inhibitory peptide concentration. Graphpad Prism 6.0 was used to calculate the inhibitory concentration of peptide required to achieve 50% maximal signal (IC50). FIG. 13 is an exemplary IC50 curve for Peptide-5 dose dependent inhibition of kinetic binding of TCR-1 to cognate MAGE-A3 pMHC.

High binding plates were first coated with neutravidin. Neutravidin coated plates were blocked using bovine serum albumin in buffer and washed. Biotinylated pMHC at a single concentration of 100 nM was captured on neutravidin coated plates, quenched using excess biocytin, and washed. Inhibitory peptide was titrated in a half-log dilution series starting from 100 uM and incubated with a constant concentration of 1 nM TCR. Inhibitory peptide and TCR mixtures were then added to the pMHC captured plates for 30 min and washed. A secondary horse radish peroxidase antibody conjugate that recognizes the TCR was then added to the plate at 1 ug/mL for 30 min and washed. Plates were then developed using tetramethylbenzidine (TMB) for 5-10 min and stopped using acid. Absorbance at 450 nm was measured for each plate and plotted versus log-scale inhibitory peptide concentration. IC50 was calculated as the concentration of peptide that inhibits 50% maximal binding signal. FIG. 14 exemplifies Peptide-5 dose dependent inhibition of TCR-1 binding to its cognate MAGE-A3 pMHC by competitive ELISA.

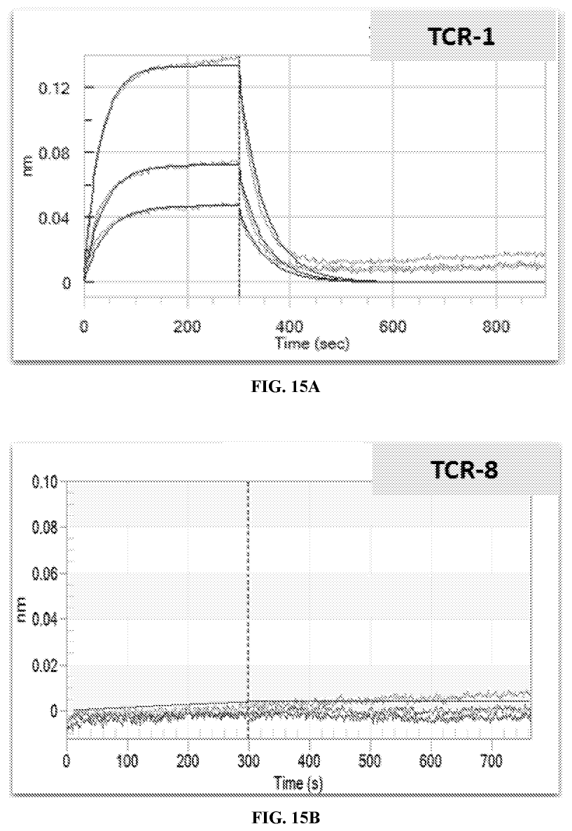

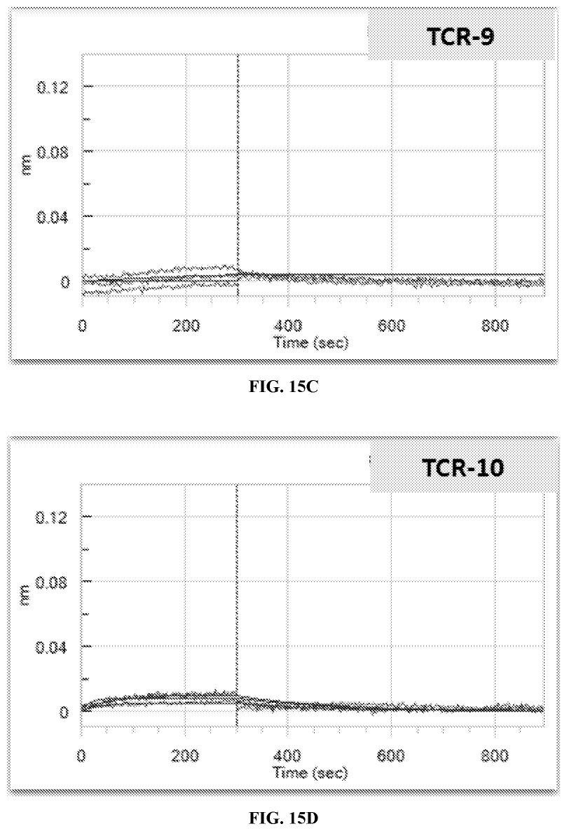

Peptide-5 binding specificity was tested against other TCRs. The other TCRS chosen are closely related to the target TCR-1. BLI based kinetic binding of TCR-land closely related TCRS, TCR-8, TCR-9, and TCR-10 to inhibitory peptides was measured using a ForteBio Octet RED96 instrument. Biotinylated peptides were first captured on streptavidin biosensors. Sensors were quenched using excess biocytin and then baselined in buffer. TCRs were titrated in a 2-fold dilution series starting from 50 nM and were associated onto the inhibitory peptide loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. Exemplary BLI sensorgrams of TCR-1, TCR-8, TCR-9, and TCR-10 TCR binding to Peptide-5 in realtime is shown in FIG. 15A-FIG. 15D. Peptide-5 is selective for TCR-1.

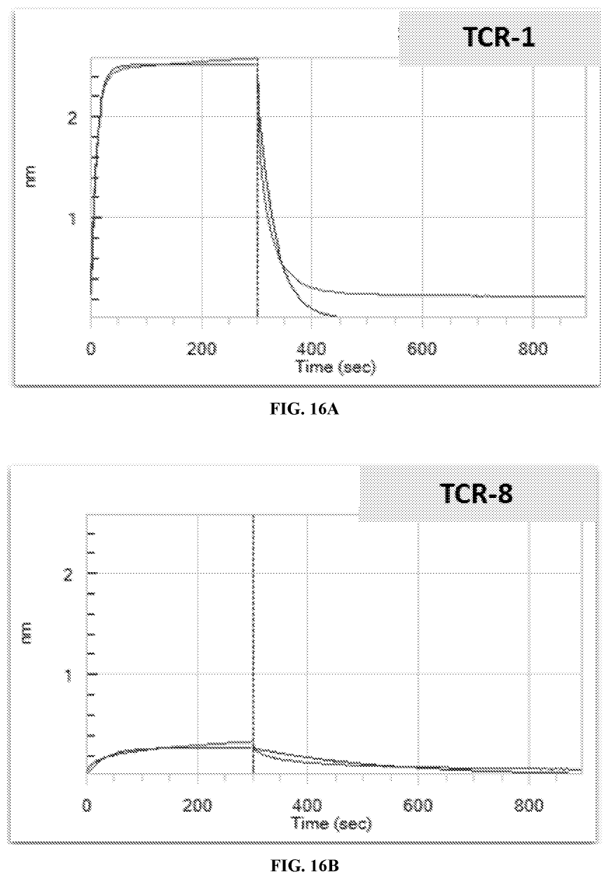

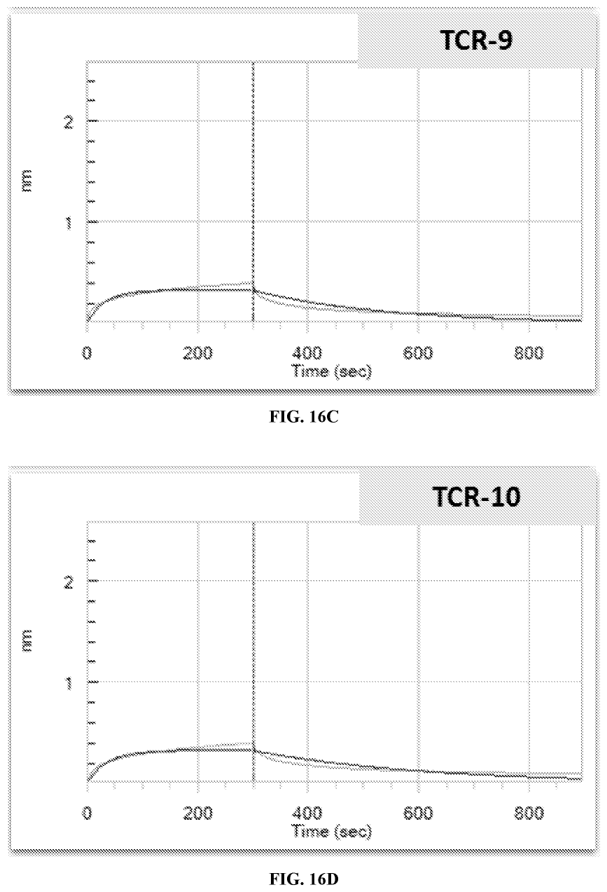

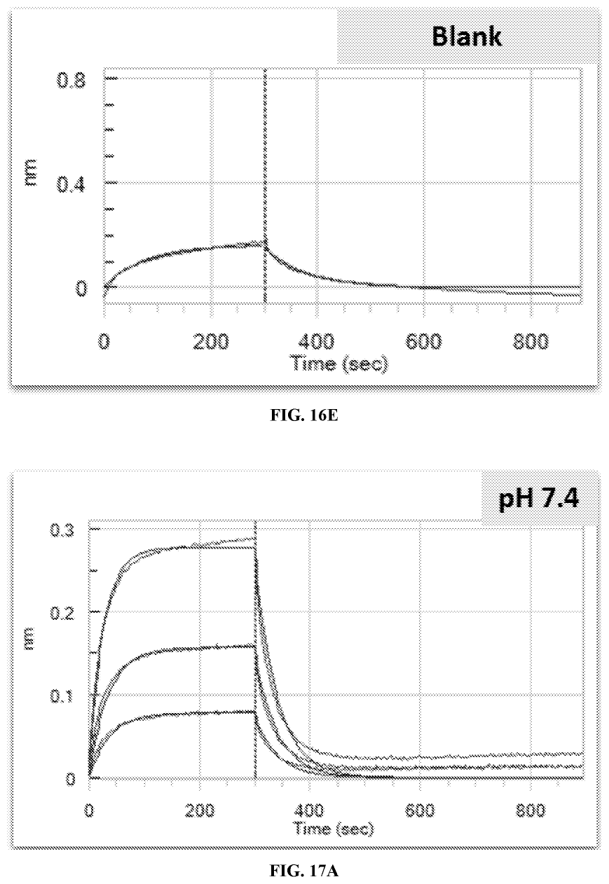

BLI based kinetic binding of TCR-land closely related TCRS, TCR-8, TCR-9, and TCR-10 to inhibitory peptides was measured using a ForteBio Octet RED96 instrument. Biotinylated peptides were first captured on streptavidin biosensors until signal saturation. Sensors were quenched using excess biocytin and then baselined in buffer. TCRs were associated 100 uM onto the inhibitory peptide loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. Exemplary BLI sensorgrams of TCR-1, TCR-8, TCR-9, and TCR-10 TCRs at 100 uM binding to saturating levels Peptide-5 loaded on streptavidin biosensors in real time is shown in FIG. 16A-FIG. 16E. Peptide-5 is selective for TCR-1.

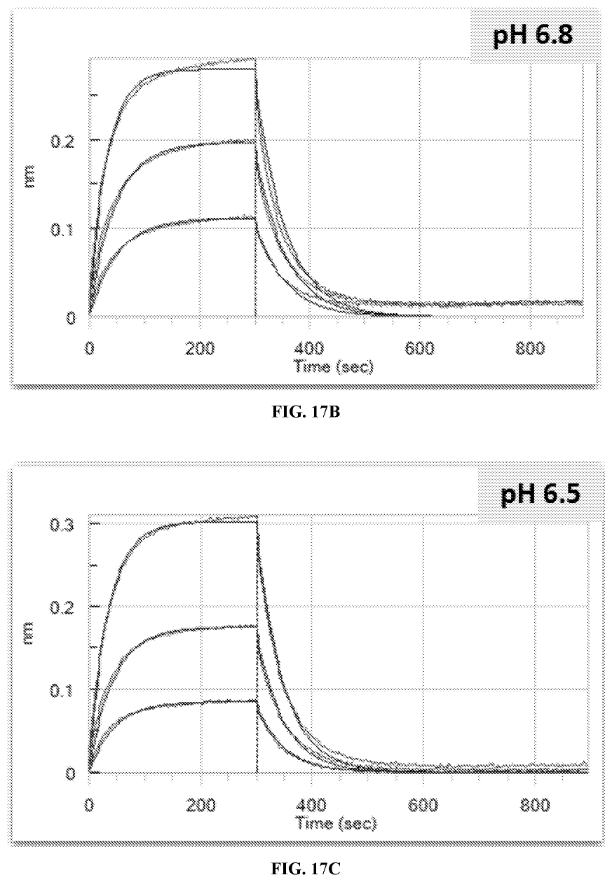

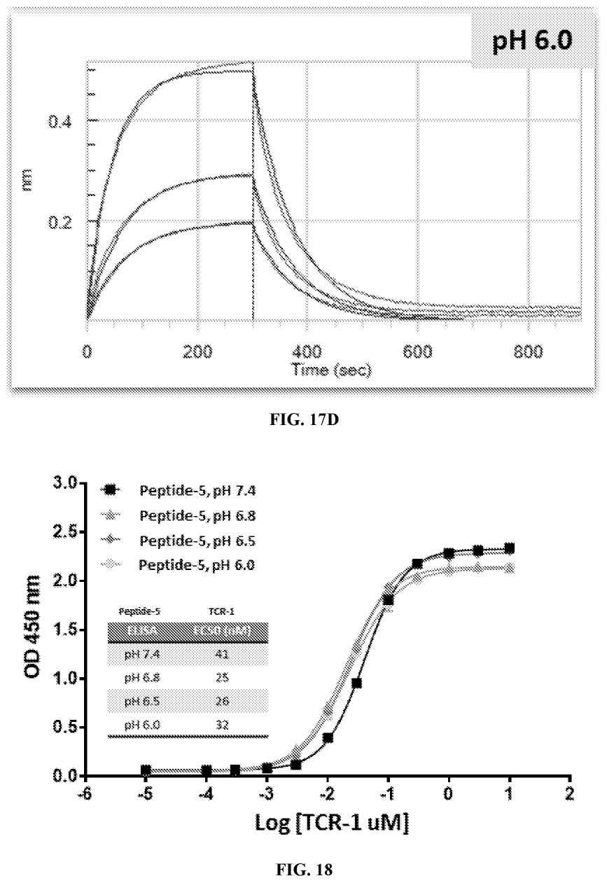

Peptide-5 dose dependent binding to TCR-1 was evaluated at acidic pH. Peptide-5 binds to TCR-1 with equal affinity at all pHs tested. BLI based kinetic binding of TCR-1 to inhibitory peptides was measured using a ForteBio Octet RED96 instrument. Biotinylated peptides were first captured on streptavidin biosensors. Sensors were quenched using excess biocytin and then baselined in buffer at desired pH. TCR-1 was diluted in buffer of desired pH and titrated in a 2-fold dilution series starting from 50 nM and was associated onto the inhibitory peptide loaded biosensor. Association signal was monitored in real-time. Biosensors were then transferred to buffer of desired pH and the dissociation of TCR was measured in real-time. Response data was baseline corrected and fit to a 1:1 binding model used to calculate association and dissociation rates. FIG. 17A-FIG. 17D exemplifies BLI sensorgrams of TCR-1 binding to Peptide-5 at acidic pH in realtime.

High binding plates were first coated with neutravidin. Neutravidin coated plates were blocked using bovine serum albumin in buffer and washed. Biotinylated inhibitory peptide at a single concentration of 100 nM was captured on neutravidin coated plates, quenched using excess biocytin, and washed. TCR was prepared in a half-log dilution series starting from 10 uM in buffer at desired pH. TCR was then titrated onto the inhibitory peptide captured plates for 1 hour and washed with buffer at desired pH. A secondary horse radish peroxidase antibody conjugate that recognizes the TCR was diluted in buffer at desired pH then added to the plate at 1 μg/mL for 1 hour and washed with buffer at desired pH. Plates were then developed using tetramethylbenzidine (TMB) for 5-10 min and stopped using acid. Absorbance at 450 nm was measured for each plate and plotted versus log-scale TCR concentration. FIG. 18 exemplifies TCR-1 binding to Peptide-5 at acidic pH by ELISA.

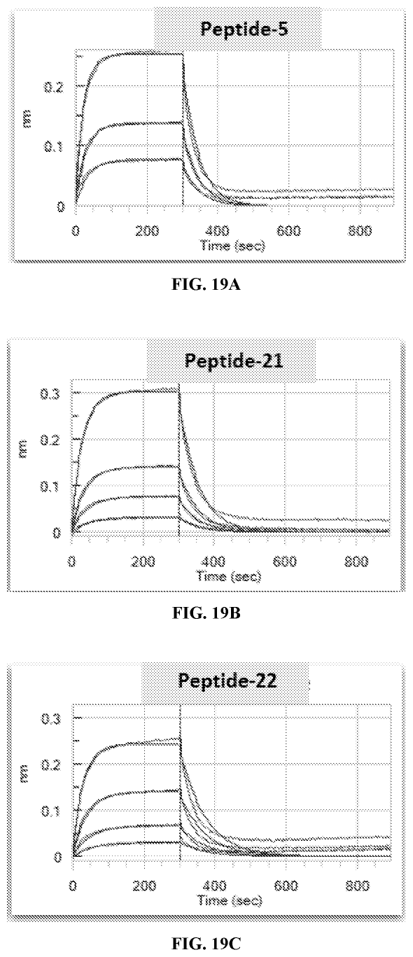

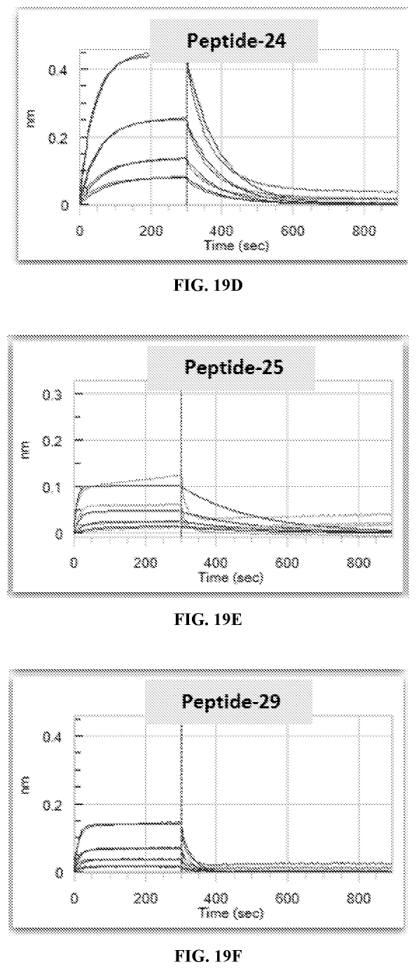

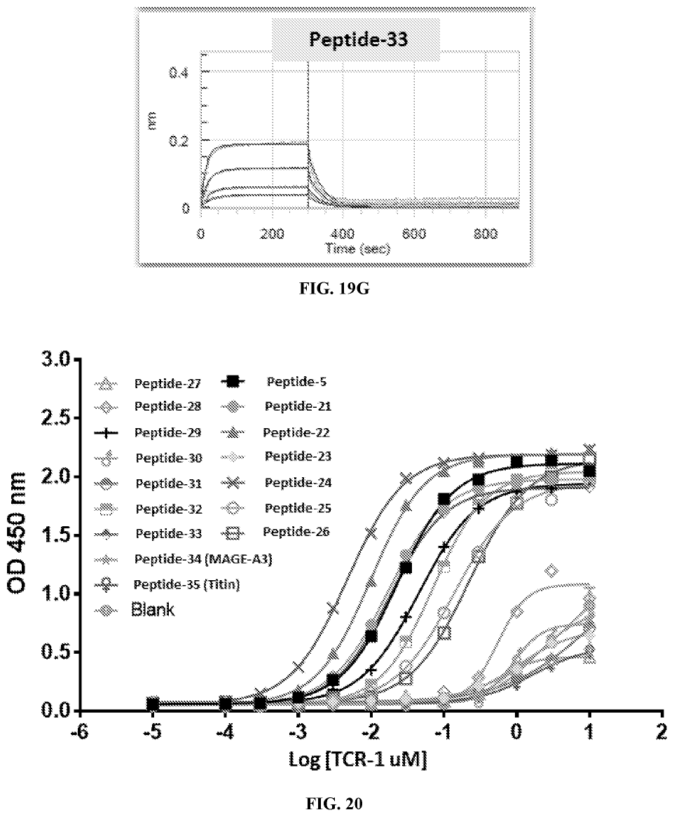

Given the strong binding data around Peptide-5 for TCR-1, we sought to better understand the key amino acid residues within the Peptide-5 sequence important for binding and functional inhibition TCR-1. Each residue within the Peptide-5 mutated one at a time to Alanine, synthesized, and evaluated as outlined. Peptide-5 Ala scan peptide data is listed in the Table 3. Peptide-5 alanine scan peptides were evaluated in kinetic binding experiments against TCR-1 as shown in FIG. 19A-FIG. 19G. Peptide-5 alanine scan peptides were evaluated for binding to TCR-1 by ELISA as shown in FIG. 20.

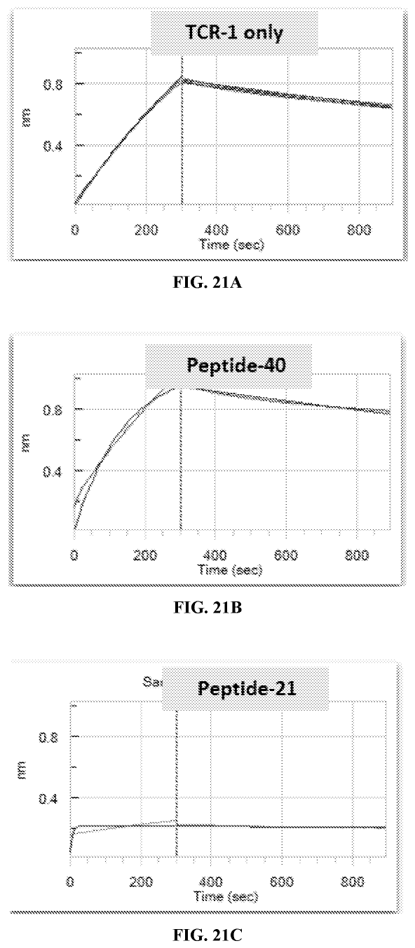

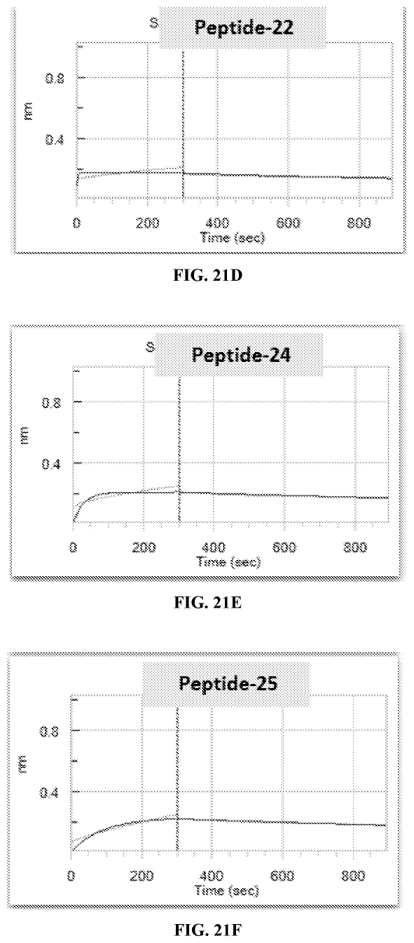

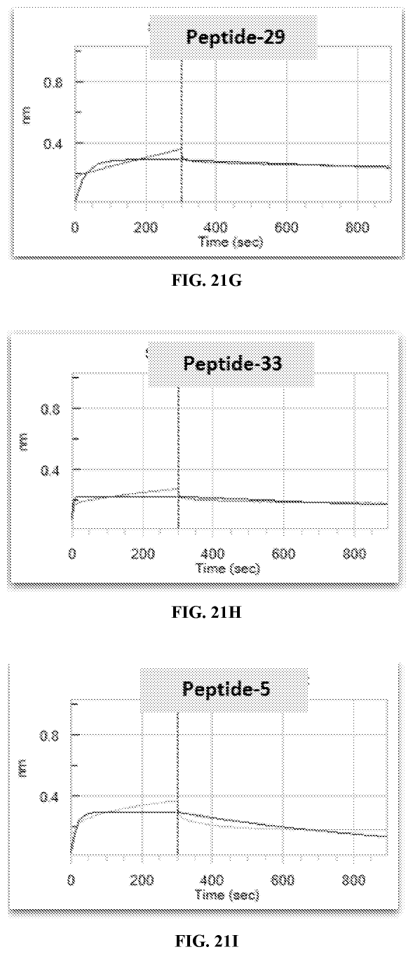

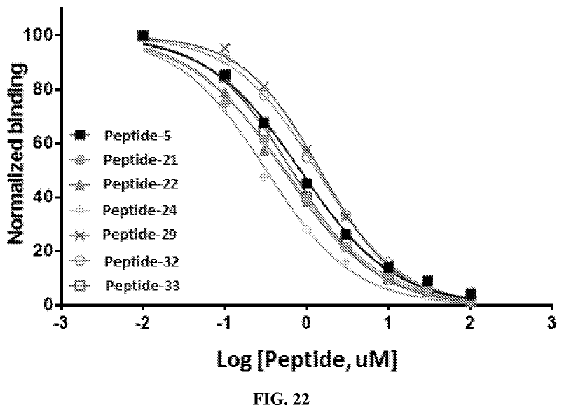

Peptide-5 alanine scan peptides were evaluated for dose dependent inhibition of TCR-1 binding to MAGE-A3 pMHC by kinetic measurements (FIG. 21A-FIG. 21I) as well as ELISA (FIG. 22). Several peptides derived from Peptide-5 bearing alanine mutations are potent binders of TCR-1. Cognate peptides MAGE-A3, Peptide-34, and Titin, Peptide-35, do not bind TCR-1 in the absence of MHC presentation. Peptides Peptide-22 and Peptide-24 show mild improvements in binding EC50. Alanine mutations at the Cysteines within Peptide-5 killed peptide activity. The cysteines are critical to maintain the cyclic nature of the peptide, a likely requirement for TCR-1 binding to Peptide-5. In general, all alanine scan peptides derived from Peptide-5 that bound to TCR-1 also maintained functional activity and blocked TCR-1 recognition of MAGE-A3 pMHC in a dose dependent fashion. Peptide-24 shows a mild improvement in competitive binding IC50.

Asp, Phe, and Cys within the Peptide-5 sequence are important for binding to TCR-1. Peptide-24 highlights potential improvement in binding by substitution of lysine at the 6th amino acid position. The alanine scan of Peptide-5 provides evidence that mutagenesis of Peptide-5 can yield more potent functional inhibitory peptides.

Peptide-5 is both a potent binder and inhibitor of TCR-1. Peptide-5 was then tethered to TCR-1 at its N-terminal beta chain or the N-terminal alpha chain using a tumor actuated protease cleavable linker. These constructs provide proof of concept that Peptide-5 tethered to TCR-1 is a functional mask in healthy tissue without protease activity that prevents TCR-1 binding to its cognate pMHC in healthy tissue. However, in tumor tissue where protease activity is high, proteases cleave the linker and release Peptide-5 from TCR-1 allowing the TCR to bind its cognate MAGE-A3 pMHC in tumor tissue. Such a construct demonstrates the tumor actuation of masked TCR-1 using Peptide-5 and a cleavable linker.

TCR-4 and TCR-5 constructs were made with a cleavable linker and Peptide-5 fused to the N-terminal beta chain of parent TCR-1. These constructs were produced recombinantly, purified, refolded and tested for binding activity and functional masking pre and post proteolysis using urokinase. Binding to cognate pMHCs was assessed by both kinetic measurements as well as ELISA.

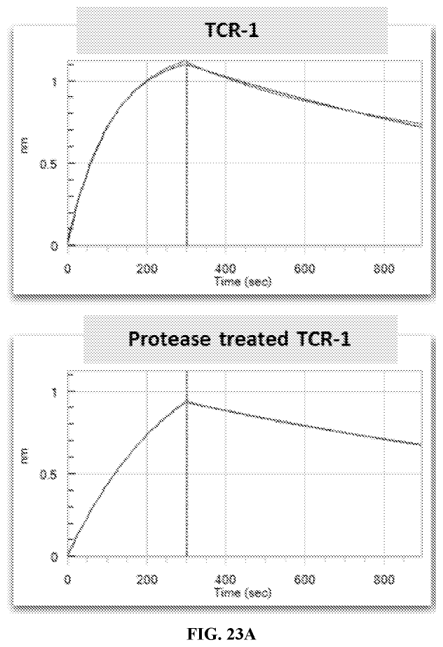

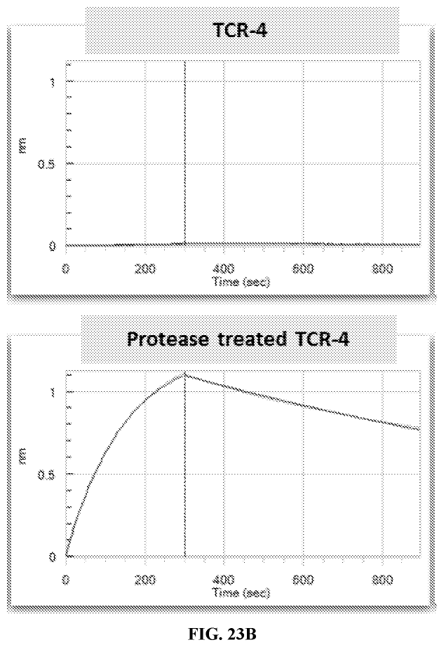

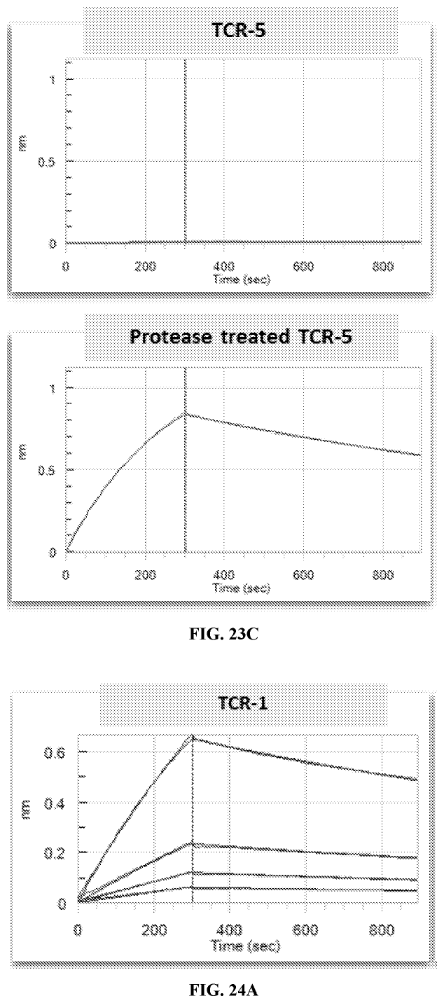

BLI based kinetic binding of TCR-1 to MAGE-A3 pMHC was measured using a ForteBio Octet RED96 instrument. Biotinylated peptides were first captured on streptavidin biosensors. Sensors were quenched using excess biocytin and then baselined in buffer. TCRs were treated with urokinase were indicated. TCRs were associated at 100 nM onto the pMHC loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. FIG. 23A-FIG. 23C exemplifies BLI sensorgrams pre and post urokinase treatment of TCR-1, TCR-4, and TCR-5 binding to MAGE-A3 pMHC in realtime. TCR-4 and TCR-5 are masked with Peptide-5 through the N-terminal beta chain of parent unmasked parent TCR, TCR-1. TCR-4 and TCR-5 differ in the length of their cleavable linker. The sequence of these masked TCR constructs are listed in Table 1. Post proteolysis, TCR-4 and TCR-5 bind equally well to cognate MAGE-A3 pMHC as parent TCR-1. However, pre proteolysis TCR-4 and TCR-5 are not capable of binding cognate MAGE-A3 pMHC.

Additional control TCRs were made to show that functional masking requires specific peptide interactions with the parent TCR. TCR-2 and TCR-3 have the TCR-1 core but are also fused to a cleavable linker of either 18 amino acids of 26 amino acids in length and inactive peptides Peptide-34 (MAGE-A3 peptide) that fails to bind TCR-1 independent of MHC presentation. TCRs TCR-2 and TCR-3 were then tested for binding to MAGE-A3 and Titin pMHCs relative to parent unmasked TCR-1. All TCRs bound to MAGE-A3 and Titin pMHC with equal affinity suggesting that Peptide-34 when fused to TCR-1 is not a functional mask. Therefore, a functional peptide mask requires binding interactions with the designed cleavable linkers to parent TCR as opposed to simple steric hindrance to inhibit TCR binding to cognate pMHC.

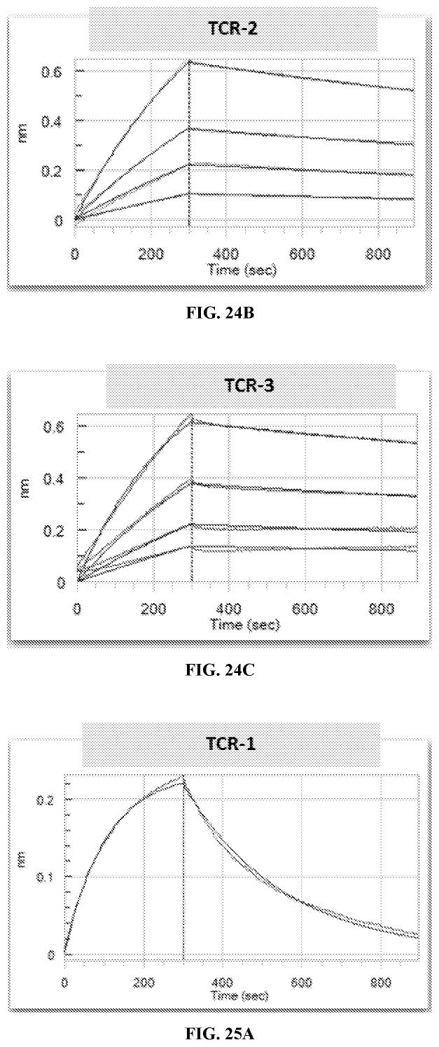

BLI based kinetic binding of TCRs to MAGE-A3 pMHC was measured using a ForteBio Octet RED96 instrument. Biotinylated peptides were first captured on streptavidin biosensors. Sensors were quenched using excess biocytin and then baselined in buffer. TCRs were treated with urokinase were indicated. TCR-1 was associated at 50 nM, 12.5 nM, 6.25 nM, and 3.125 nM while TCR-2 and TCR-3 were associated at 50 nM, 25 nM, 12.5 nM, and 6.25 nM onto the pMHC loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. FIG. 24A-FIG. 24C exemplifies BLI sensorgrams of TCR-1, TCR-2, and TCR-3 binding to MAGE-A3 pMHC in realtime. TCRs tethered to natural MAGE-A3 peptide binds cognate pMHC complex equally well as unmasked TCR, TCR-1. MAGE-A3 peptide, Peptide-34 is not capable of masking TCR-1. TCR-2 and TCR-3 are masked with Peptide-34 through the N-terminal beta chain of parent unmasked TCR, TCR-1.

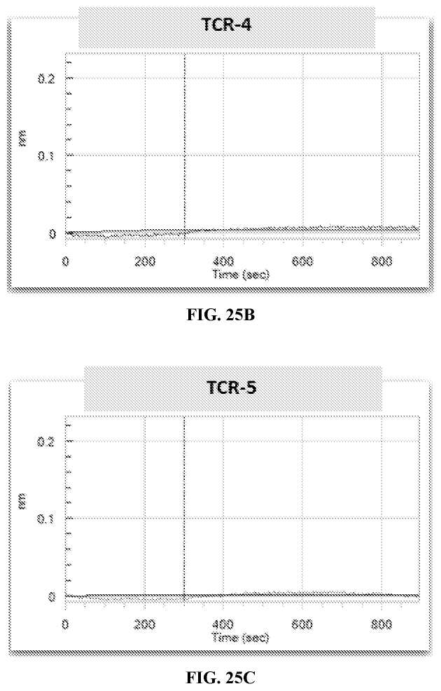

BLI based kinetic binding of TCR-1 to Titin pMHC was measured using a ForteBio Octet RED96 instrument. Biotinylated peptides were first captured on streptavidin biosensors. Sensors were quenched using excess biocytin and then baselined in buffer. TCRs were treated with urokinase were indicated. TCRs were associated at 100 nM onto the pMHC loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. FIG. 25A-FIG. 25C exemplifies BLI sensorgrams of TCR-1, TCR-4 and TCR-5 binding to Titin pMHC in realtime. Peptide-5 masking of TCR protects against known undesirable healthy tissue binding of Titin-pMHC in vitro. TCR-4 and TCR-5 are masked with Peptide-5 through the N-terminal beta chain of parent unmasked TCR, TCR-1.

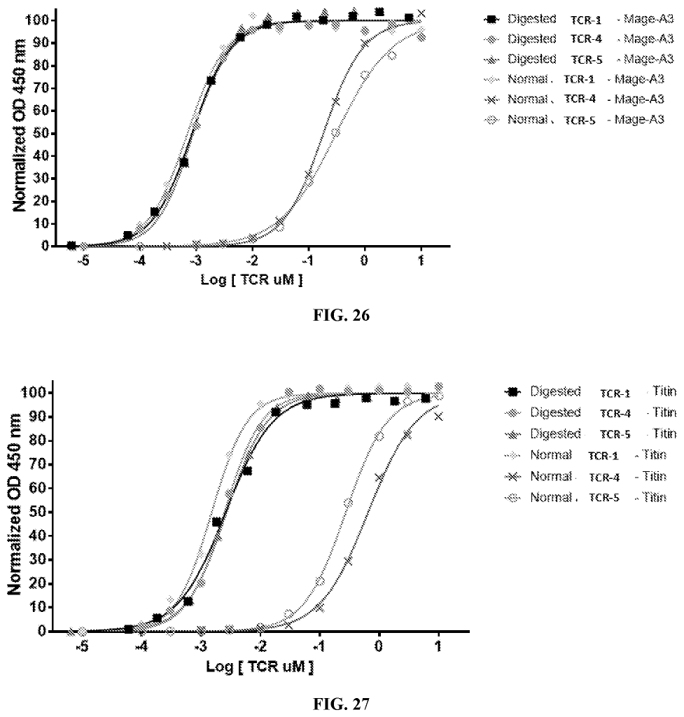

High binding plates were first coated with neutravidin. Neutravidin coated plates were blocked using bovine serum albumin in buffer and washed. Biotinylated MAGE-A3 pMHC at a single concentration of 100 nM was captured on neutravidin coated plates, quenched using excess biocytin, and washed. Masked TCRs were digested with human recombinant urokinase as indicated. TCRs were then prepared in a half-log dilution series starting from 10 uM. TCR was then titrated onto the pMHC captured plates for 1 hour and washed. A secondary horse radish peroxidase antibody conjugate that recognizes the TCR was then added to the plate at 1 μg/mL for 1 hour and washed. Plates were then developed using tetramethylbenzidine (TMB) for 5-10 min and stopped using acid. Absorbance at 450 nm was measured for each plate and plotted versus log-scale TCR concentration. FIG. 26 exemplifies binding of Peptide-5 masked TCRs with a cleavable linker, TCR-4 and TCR-5, relative to unmasked TCR, TCR-1, to MAGE-A3 pMHC. TCRs were tested pre and post urokinase digestion. TCR-4 and TCR-5 are masked with Peptide-5 through the N-terminal beta chain of parent unmasked TCR, TCR-1.

High binding plates were first coated with neutravidin. Neutravidin coated plates were blocked using bovine serum albumin in buffer and washed. Biotinylated Titin pMHC at a single concentration of 100 nM was captured on neutravidin coated plates, quenched using excess biocytin, and washed. Masked TCRs were digested with human recombinant urokinase as indicated. TCRs were then prepared in a half-log dilution series starting from 10 uM. TCR was then titrated onto the pMHC captured plates for 1 hour and washed. A secondary horse radish peroxidase antibody conjugate that recognizes the TCR was then added to the plate at 1 μg/mL for 1 hour and washed. Plates were then developed using tetramethylbenzidine (TMB) for 5-10 min and stopped using acid. Absorbance at 450 nm was measured for each plate and plotted versus log-scale TCR concentration. FIG. 27 exemplifies binding of Peptide-5 masked TCRs with a cleavable linker, TCR-4 and TCR-5, relative to unmasked TCR, TCR-1, to Titin pMHC. TCRs were tested pre and post urokinase digestion. TCR-4 and TCR-5 are masked with Peptide-5 through the N-terminal beta chain of parent unmasked TCR, TCR-1.

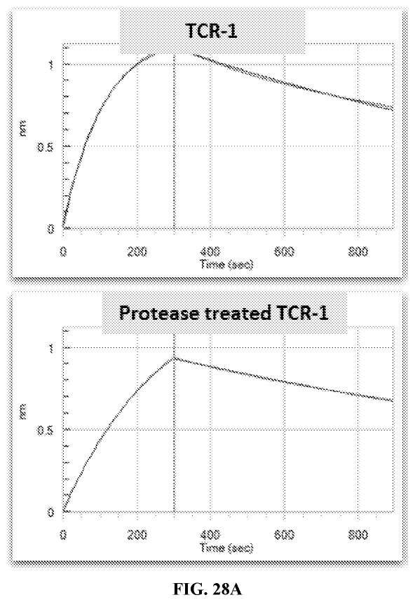

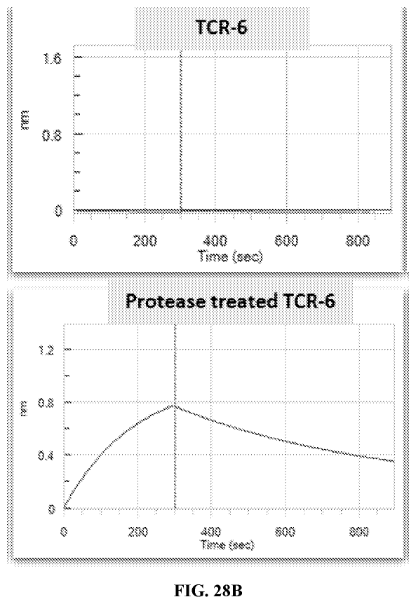

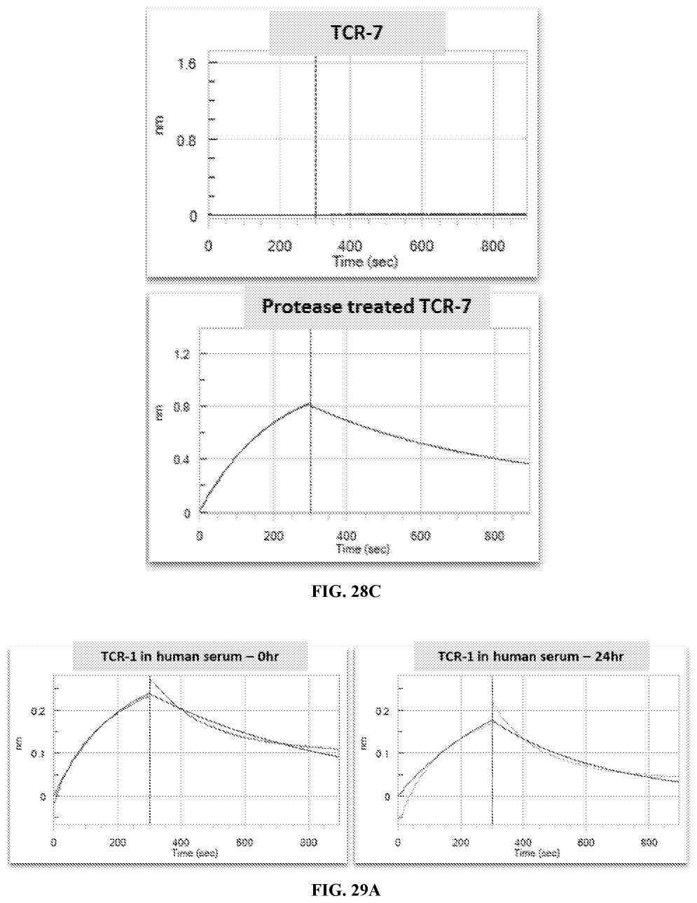

TCR-6 and TCR-7 constructs were made with a cleavable linker and Peptide-5 fused to the N-terminal alpha chain of parent TCR-1. These constructs were produced recombinantly, purified, refolded and tested for binding activity and functional masking pre and post proteolysis using urokinase. Binding to MAGE-A3 pMHC was assessed by kinetic measurements. FIG. 28A-FIG. 28C exemplifies BLI sensorgrams pre and post urokinase treatment of TCR-1, TCR-6 and TCR-7 binding to MAGE-A3 pMHC in realtime. TCR-6 and TCR-7 are masked with Peptide-5 through the N-terminal alpha chain of parent unmasked TCR, TCR-1. BLI based kinetic binding of TCR-1 to MAGE-A3 pMHC was measured using a ForteBio Octet RED96 instrument. Biotinylated peptides were first captured on streptavidin biosensors. Sensors were quenched using excess biocytin and then baselined in buffer. TCRs were treated with urokinase were indicated. TCRs were associated at 100 nM onto the pMHC loaded biosensor. Association signal was monitored in real-time. Biosensors were then transferred to buffer and the dissociation of TCR was measured in real-time. Response data was baseline corrected and fit to a 1:1 binding model used to calculate association and dissociation rates.

Serum stability of the cleavable linker and Peptide-5 fusions were determined by incubating TCR-5 as well as parent unmasked TCR-1 in human serum at 37 C for 24 hours. After 24 hours in warm serum, TCRs were diluted in assay buffer and tested for kinetic binding activity against MAGE-A3 pMHC. While parent TCR-1 maintains binding affinity after 24 hours in serum, TCR-5 maintains functional masking and lacks binding to MAGE-A3 pMHC. This indicates that not only is the parent unmasked TCR stable in human serum for >24 hours, but that the cleavable linker and Peptide-5 fusions maintain their function as masks for >24 hours in human serum as well. Similar data was generated for mouse serum.

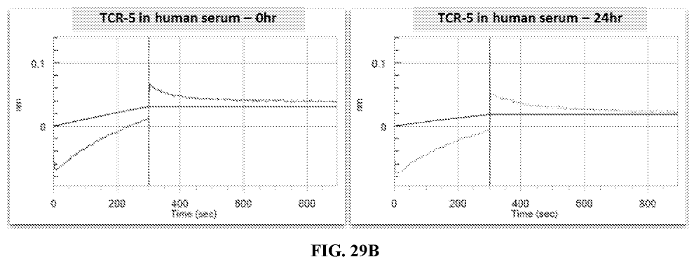

BLI based kinetic binding of TCR-1 to MAGE-A3 pMHC was measured using a ForteBio Octet RED96 instrument. Biotinylated peptides were first captured on streptavidin biosensors. Sensors were quenched using excess biocytin and then baselined in buffer. TCRs were treated with urokinase were indicated. TCRs were associated at 100 nM onto the inhibitory peptide loaded biosensor. Association signal was monitored in real-time. Biosensors were then transferred to buffer and the dissociation of TCR was measured in real-time. Response data was baseline corrected and fit to a 1:1 binding model used to calculate association and dissociation rates. FIG. 29A-FIG. 29B exemplifies BLI sensorgrams of TCR-1, TCR-4, or TCR-5 binding to cognate MAGE-A3 pMHC pre and post 24 hour incubation in human serum. TCR-4 and TCR-5 are masked with Peptide-5 through the N-terminal beta chain of parent unmasked TCR, TCR-1. TCR-1 remains stable and maintains binding to MAGE-A3 in human serum for >24 hours. Peptide-5 and linker maintain inhibition of TCR binding to cognate pMHC and therefore remain functionally stable in human serum for >24 hours.

### Example 10. TCR-8 Peptide Library Biopanning

Biopanning with m13 phagemid p8 or p3 displayed peptide libraries was either performed with directly coated T cell receptor, TCR-8 (Table 2) on 96-well ELISA plates or with biotin-conjugated T cell receptors immobilized on streptavidin coated paramagnetic beads. Following binding to target and washing steps, specifically bound phage were recovered by elution at pH 2.2. Enrichment of specific binding clones was generally accomplished by 3-4 rounds of successive biopanning and amplification. After 3 or 4 rounds of biopanning the resulting phage pools were infected into TG1 cells and plated out on LB-ampicillin/agar plates for clonal isolation and subsequent characterization.

### Example 11. Phagemid TCR-8 Hit Identification ELISA

For hit identification, individual colonies were grown in 96-deep well plates for 2-4 hours and infected with helper phage and induced to produce peptide displayed phagemid following an overnight growth. The next day the deep well plates were centrifuged to separate the soluble phagemid from the E. coli cells. The phagemid containing supernatants were then combined with PBS-Tween 20 (0.05%)+BSA (1%) blocking buffer and incubated in ELISA wells containing Neutravidin captured biotin-conjugated TCR-8 (Table 1) or Neutravidin alone. After binding at 4 degrees the plates were washed and specifically bound phage were detected by anti-m13 HRP conjugated antibodies using standard TMB-based chromogenic ELISA procedures. Daughter plates or individual wells were subjected to standard DNA sequencing for clonal peptide sequence identification. Table 4 below shows the quantitative ELISA results for phagemid binding to IC-3 and peptide sequence identity of the respective unique clones.

### Example 12. TCR-8 Phagemid Competition ELISA Assay

Phagemid peptide clones that specifically bound TCR-1 were next tested to determine whether they bound within the antigen binding space of the T cell receptor, by target-based competition assay. TCR-8 immobilized and blocked 96-well ELISA plates similar to above were prepared. Tetrameric human MAGE-A3 pMHC was first added to wells to block the active binding site. After a brief incubation period (30-60 minutes) phagemid supernatants were added to both sets of wells. Following further incubation at 4 degrees the plates were washed and specifically bound phage were detected by anti-m13 HRP conjugated antibodies using standard TMB-based chromogenic ELISA procedures. Phagemid clones binding within the antigenic binding pocket would be blocked and be identified by a decreased ELISA signal, compared to a well lacking previous pMHC blockade.

Table 4 below shows the ELISA results for the pMHC competition of phagemid binding to TCR-8, along with primary ELISA results and sequence identities of the respectively tested phagemid clones.

TABLE 4
Peptide Phagemid TCR-8 binding ELISA data,
and peptide sequence for each of the clones.

| | | | Secondary pMHC phagemid | | | | |
|---|---|---|---|---|---|---|---|
| | Primary phagemid | | competition ELISA | | | | |
| | binding ELISA | | TCR-8 | binding with | | | |
| | | Netravidin | binding no | 100 nM pMHC | % binding | | |
| | TCR-8 | Control | pMHC | tetramer | with pMHC | SEQ | |
| clone | wells | wells | competitor | competitor | competition | ID NO: | peptide sequence |
| J297_A02 | 3.153 | 0.135 | 1.395 | 0.190 | 14% | 134 | GGRVCYFDHQVVHCIW |
| J297_A05 | 3.075 | 0.099 | 0.294 | 0.057 | 19% | 165 | GGIWCWFENQSVICTA |
| J297_A11 | 3.118 | 0.100 | 0.859 | 0.139 | 16% | 153 | GGVICWFQEGMVHCVS |
| J297_B02 | 3.118 | 0.155 | 0.435 | 0.058 | 13% | 166 | GGIICWFQSFEVYCMG |
| J297_B09 | 2.937 | 0.083 | 0.262 | 0.095 | 36% | 167 | GGIICWFEVGQVRCQD |
| J297_C12 | 2.087 | 0.129 | 0.454 | 0.133 | 29% | 168 | GGCFSYSPWGTTWSHC |
| J297_H10 | 3.242 | 0.137 | 0.430 | 0.404 | 94% | 169 | GGVCFVLPWPQLKLVC |
| J306_A01 | 3.152 | 0.126 | 1.241 | 0.637 | 51% | 108 | GGACVAENEWAYWYCR |
| J306_E07 | 3.178 | 0.125 | 1.873 | 0.309 | 17% | 170 | GGRGCYFDHQVVHCIW |
| J307_A03 | 3.154 | 0.084 | 1.588 | 0.164 | 10% | 171 | GGYLCWFEGGGLVSCA |
| J307_A08 | 3.134 | 0.240 | 1.935 | 0.474 | 24% | 172 | GGSVCFFKGGGLVICY |
| J307_B02 | 3.155 | 0.074 | 1.541 | 0.211 | 14% | 173 | GGLSCWFAGYARVECA |
| J307_B07 | 3.182 | 0.076 | 1.354 | 0.232 | 17% | 174 | GGVECWFAGGGDVICA |
| J307_B09 | 3.129 | 0.211 | 1.821 | 0.443 | 24% | 175 | GGRFCWFAAFSTVLCV |
| J307_C02 | 3.180 | 0.186 | 2.273 | 0.595 | 26% | 176 | GGITCFFYPAHMVTCS |
| J307_C07 | 3.103 | 0.150 | 0.876 | 0.070 | 8% | 177 | GGYFCWFSGEKAVICS |
| J307_C12 | 3.152 | 0.141 | 1.692 | 0.297 | 18% | 178 | GGMVCFFEGRGQVVCI |
| J307_D07 | 3.175 | 0.124 | 1.838 | 0.360 | 20% | 179 | GGIFCWFVGSSTVTCE |
| J307_E08 | 3.146 | 0.061 | 0.954 | 0.101 | 11% | 180 | GGYGCLGGLWDYWYCA |
| J307_G12 | 3.174 | 0.143 | 1.576 | 0.382 | 24% | 181 | GGYLCFFEGGGLVSCA |
| J307_H03 | 2.800 | 0.067 | 1.528 | 0.344 | 23% | 182 | GGISCWFSGVGQVLCY |
| J307_H06 | 3.172 | 0.108 | 0.774 | 0.064 | 8% | 183 | GGYLCCFEGGGLVSCA |
| J308_A03 | 3.157 | 0.132 | 1.654 | 0.358 | 22% | 184 | GGVICENWQGDRVCWF |
| J308_A08 | 3.147 | 0.220 | 1.716 | 0.341 | 20% | 185 | GGYFCYFESSMSHCLY |
| J308_A09 | 3.065 | 0.236 | 1.877 | 0.570 | 30% | 184 | GGVICENWQGDRVCWF |
| J308_B04 | 3.154 | 0.101 | 1.747 | 0.420 | 24% | 186 | GGIFCWFQDFSVYCKS |
| J308_B12 | 3.113 | 0.180 | 2.298 | 0.521 | 23% | 187 | GGYVCYFYNASVTCVY |
| J308_D01 | 3.273 | 0.206 | 2.013 | 0.478 | 24% | 188 | GGQMCHFEYNLVVCYH |
| J308_D04 | 3.178 | 0.156 | 1.624 | 0.305 | 19% | 189 | GGCYFDFGVLGTSVVC |
| J308_D09 | 3.184 | 0.061 | 0.920 | 0.077 | 8% | 190 | GGRMCHFDVNTVVCYL |
| J308_E03 | 3.200 | 0.119 | 1.408 | 0.266 | 19% | 191 | GGSVCYFELSVVICVN |
| J308_E07 | 3.181 | 0.189 | 1.622 | 0.350 | 22% | 131 | GGYACWFGHGLVHCGT |
| J308_F01 | 3.300 | 0.281 | 2.119 | 0.688 | 32% | 192 | GGRTCYFDQGSVVCYW |
| J308_F09 | 3.182 | 0.166 | 1.900 | 0.386 | 20% | 193 | GGVFCWFEWSVVTCSH |
| J308_G03 | 3.212 | 0.205 | 1.616 | 0.370 | 23% | 194 | GGYMCYFSMKTVVCQW |
| J308_G06 | 3.211 | 0.099 | 1.281 | 0.245 | 19% | 195 | GGDFCWFFNREVLCYG |
| J308_H12 | 3.185 | 0.200 | 1.275 | 0.111 | 9% | 196 | GGTFCYFVNFSVTCVN |
| J309_C11 | 2.403 | 0.163 | 0.655 | 0.058 | 9% | 197 | GGSVNCIDAIFACFLV |

### Certain Embodiments

Embodiment 1 provides a modified T cell receptor (TCR) comprising a polypeptide of formula I: T₁-L₁-P₁ (formula I) wherein: T₁ comprises a transmembrane domain and either a TCR alpha extracellular domain, or a fragment thereof, or a TCR beta extracellular domain, or a fragment thereof, wherein T₁ binds to a target antigen and the TCR alpha extracellular domain or fragment thereof and the TCR beta extracellular domain, or fragment thereof contain an antigen binding site, P₁ is a peptide that reduces binding of T₁ to the target antigen when the modified TCR is outside of a tumor microenvironment and that does not reduce binding of T₁ to the target antigen when the modified TCR is inside the tumor microenvironment, and L₁ is a linking moiety that connects T₁ to P₁ and L₁ is bound to T₁ at the N-terminus of T₁, wherein the modified TCR is a functional TCR when inside the tumor microenvironment and is a nonfunctional TCR when outside the tumor microenvironment and P₁ or L₁ is a substrate for a tumor specific protease.

Embodiment 2 provides the modified TCR of embodiment 1, wherein P₁ is bound to T₁ through ionic interactions, electrostatic interactions, hydrophobic interactions, Pi-stacking interactions, and H— bonding interactions, or a combination thereof when the modified TCR is outside the tumor microenvironment.

Embodiment 3 provides the modified TCR of any one of embodiments 1-2, wherein P₁ is bound to T₁ at or near the antigen binding site when the modified TCR is outside the tumor microenvironment.

Embodiment 4 provides the modified TCR of any one of embodiments 1-3, wherein P₁ inhibits the binding of T₁ to the target antigen when the modified TCR is outside the tumor microenvironment, and P₁ does not inhibit the binding of T₁ to the target antigen when the modified TCR is inside the tumor microenvironment.

Embodiment 5 provides the modified TCR of any one of embodiments 1-4, wherein P₁ sterically blocks T₁ from binding to the target antigen when the modified TCR is outside the tumor microenvironment.

Embodiment 6 provides the modified TCR of any one of embodiments 3-5, wherein P₁ is removed from the antigen binding site, and the antigen binding site of T₁ is exposed when the modified TCR is inside the tumor microenvironment.

Embodiment 7 provides the modified TCR of any one of embodiments 1-6, wherein P₁ comprises at least 70% sequence homology to the target antigen.

Embodiment 8 provides the modified TCR of any one of embodiments 1-7, wherein P₁ is a substrate for a tumor specific protease.

Embodiment 9 provides the modified TCR of any one of embodiments 1-7, wherein the tumor specific protease is selected from the group consisting of metalloprotease, serine protease, cysteine protease, threonine protease, and aspartic protease.

Embodiment 10 provides the modified TCR of any one of embodiments 1-8, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 11 provides the modified TCR of any one of embodiments 1-10, wherein P₁ comprises a peptide sequence of at least 6 amino acids in length.

Embodiment 12 provides the modified TCR of any one of embodiments 1-11, wherein P₁ comprises a peptide sequence of at least 10 amino acids in length.

Embodiment 13 provides the modified TCR of any one of embodiments 1-11, wherein P₁ comprises a linear or cyclic peptide.

Embodiment 14 provides the modified TCR of any one of embodiments 1-13, wherein P₁ comprises a modified amino acid, a non-natural amino acid, or a modified non-natural amino acids, or combination thereof.

Embodiment 15 provides the modified TCR of embodiment 14, wherein the modified amino acid or modified non-natural amino acid comprises a post-translational modification.

Embodiment 16 provides the modified TCR of any one of embodiments 1-15, wherein L₁ is a peptide sequence having at least 5 to no more than 50 amino acids.

Embodiment 17 provides the modified TCR of any one of embodiments 1-16, wherein L₁ has a formula selected from the group consisting of: (GS)ₙ, wherein n is an integer from 6 to 20 (SEQ ID NO: 1); (G₂S)ₙ, wherein n is an integer from 4 to 13 (SEQ ID NO: 2); (G₃S)ₙ, wherein n is an integer from 3 to 10 (SEQ ID NO: 3); and (G₄S)ₙ, wherein n is an integer from 2 to 8 (SEQ ID NO: 4); and (G)ₙ, wherein n is an integer from 12 to 40 (SEQ ID NO: 5).

Embodiment 18 provides the modified TCR of any one of embodiments 1-16, wherein L₁ has a formula comprising (GGSGGD)ₙ, wherein n is an integer from 2 to 6 (SEQ ID NO: 8).

Embodiment 19 provides the modified TCR of any one of embodiments 1-16, wherein L₁ has a formula comprising (GGSGGE)ₙ, wherein n is an integer from 2 to 6 (SEQ ID NO: 9).

Embodiment 20 provides the modified TCR of any one of embodiments 1-16, wherein L₁ has a formula comprising (GGGSGSGGGGS)ₙ, wherein n is an integer from 1 to 3 (SEQ ID NO: 6).

Embodiment 21 provides the modified TCR of any one of embodiments 1-16, wherein L₁ has a formula comprising (GGGGGPGGGGP)ₙ, wherein n is an integer from 1 to 3 (SEQ ID NO: 7).

Embodiment 22 provides the modified TCR of any one of embodiments 1-16, wherein L₁ has a formula selected from: (GX)ₙ, wherein X is serine, aspartic acid, glutamic acid, threonine, or proline and n is at least 20 (SEQ ID NO: 24); (GGX)ₙ, wherein X is serine, aspartic acid, glutamic acid, threonine, or proline and n is at least 13 (SEQ ID NO: 25); (GGGX)ₙ, wherein X is serine, aspartic acid, glutamic acid, threonine, or proline and n is at least 10 (SEQ ID NO: 26); (GGGGX)ₙ, wherein X is serine, aspartic acid, glutamic acid, threonine, or proline and n is at least 8 (SEQ ID NO: 27); and (G_{z}X)ₙ, wherein X is serine, aspartic acid, glutamic acid, threonine, or proline and n is at least 15, and z is between 1 and 20 (SEQ ID NO: 28).

Embodiment 23 provides the modified TCR of any one of embodiments 1-16, wherein L₁ is a substrate for a tumor specific protease.

Embodiment 24 provides the modified TCR of embodiment 23, wherein the tumor specific protease is selected from the group consisting of metalloprotease, serine protease, cysteine protease, threonine protease, and aspartic protease.

Embodiment 25 provides the modified TCR of embodiment 23, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 26 provides the modified TCR of any one of embodiments 1-16, wherein L₁ comprises a plasmin cleavable amino acid sequence.

Embodiment 27 provides the modified TCR of embodiment 26, wherein the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15).

Embodiment 28 provides the modified TCR of any one of embodiments 1-16, wherein L₁ comprises a Factor Xa cleavable amino acid sequence.

Embodiment 29 provides the modified TCR of embodiment 28, wherein the Factor Xa cleavable amino acid sequence is selected from the group consisting of IEGR (SEQ ID NO: 16), IDGR (SEQ ID NO: 17), and GGSIDGR (SEQ ID NO: 18).

Embodiment 30 provides the modified TCR of any one of embodiments 1-16, wherein L₁ comprises an MMP cleavable amino acid sequence.

Embodiment 31 provides the modified TCR of embodiment 30, wherein the MMP cleavable amino acid sequence is PLGLWA (SEQ ID NO: 19).

Embodiment 32 provides the modified TCR of any one of embodiments 1-16, wherein L₁ comprises a collagenase cleavable amino acid sequence.

Embodiment 33 provides the modified TCR of embodiment 32, wherein the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

Embodiment 34 provides the modified TCR of any one of embodiments 1-33, wherein L₁ comprises a modified amino acid.

Embodiment 35 provides the modified TCR of embodiment 34, wherein the modified amino acid comprises a post-translational modification.

Embodiment 36 provides the modified TCR of any one of embodiments 1-35, wherein L₁ comprises a non-natural amino acid or a modified non-natural amino acid, or combination thereof.

Embodiment 37 provides the modified TCR of embodiment 36, wherein the modified non-natural amino acid comprises a post-translational modification.

Embodiment 38 provides the modified TCR of any one of embodiments 1-34, wherein the target antigen is selected from the group consisting of MAGE-A3, NY-ESO-1, gp100, WT1, and tyrosinase.

Embodiment 39 provides the modified TCR of any one of embodiments 1-38, wherein T₁ comprises the TCR alpha extracellular domain, or fragment thereof, and the modified TCR further comprises a second polypeptide comprising a transmembrane domain and a TCR beta extracellular domain, or fragment thereof wherein the TCR beta extracellular domain or fragment thereof contains an antigen binding site.

Embodiment 40 provides the modified TCR of any one of embodiments 1-38, wherein T₁ comprises the TCR beta extracellular domain, or fragment thereof, and the modified TCR further comprises a second polypeptide comprising a transmembrane domain and a TCR alpha extracellular domain, or fragment thereof wherein the TCR alpha extracellular domain or fragment thereof contains an antigen binding site.

Embodiment 41 provides the modified TCR of any one of embodiments 1-38, wherein T₁ comprises the TCR alpha extracellular domain, or fragment thereof, and the modified TCR further comprises a second polypeptide of formula II: T₂-L₂-P₂ (formula II) wherein T₂ comprises a transmembrane domain and a TCR beta extracellular domain, or fragment thereof, wherein T₂ binds to a target antigen and the TCR beta extracellular domain or fragment thereof contains an antigen binding site, P₂ is a peptide that reduces binding of T₂ to the target antigen when the modified TCR is outside of a tumor microenvironment and that does not reduce binding of T₂ to the target antigen when the modified TCR is inside the tumor microenvironment, and L₂ is a linking moiety that connects T₂ to P₂ and L₂ is bound to T₂ at the N-terminus of T₂, wherein P₂ or L₂ is a substrate for a tumor specific protease.

Embodiment 42 provides the modified TCR of any one of embodiments 39-41, wherein TCR alpha extracellular domain, or fragment thereof, and the TCR beta extracellular domain, or fragment thereof, are connected by a disulfide bond.

Embodiment 43 provides the modified TCR of embodiment 40, wherein the TCR alpha extracellular domain, or fragment thereof, comprises an alpha chain TRAC constant domain sequence and the TCR beta extracellular domain or fragment thereof, comprises a beta chain TRBC1 or TRBC2 constant domain sequence.

Embodiment 44 provides the modified TCR of embodiment 39, wherein T₁ comprises the TCR beta extracellular domain, or a fragment thereof, and the modified TCR further comprises a second polypeptide comprising a transmembrane domain and a TCR alpha extracellular domain, or a fragment thereof wherein the TCR alpha extracellular domain or fragment thereof contains an antigen binding site and the polypeptide and the second polypeptide are connected by a disulfide bond.

Embodiment 45 provides the modified TCR of any one of embodiments 39-44, wherein T₁ comprises the TCR alpha extracellular domain, or a fragment thereof, and the modified TCR further comprises a second polypeptide comprising a transmembrane domain and a TCR beta extracellular domain, or a fragment thereof wherein the TCR beta extracellular domain or fragment thereof contains an antigen binding site and the polypeptide and the second polypeptide are connected by a disulfide bond.

Embodiment 46 provides the modified TCR of any one of embodiments 41-45, wherein P₂ is bound to T₂ through ionic interactions, electrostatic interactions, hydrophobic interactions, Pi-stacking interactions, and H-bonding interactions, or a combination thereof when the modified TCR is outside the tumor microenvironment.

Embodiment 47 provides the modified TCR of any one of embodiments 41-46, wherein P₂ is bound to T₂ at or near the antigen binding site when the modified TCR is outside the tumor microenvironment.

Embodiment 48 provides the modified TCR of any one of embodiments 41-47, wherein P₂ inhibits the binding of T₂ to the target antigen when the modified TCR is outside the tumor microenvironment, and P₂ does not inhibit the binding of T₂ to the target antigen when the modified TCR is inside the tumor microenvironment.

Embodiment 49 provides the modified TCR of any one of embodiments 41-48, wherein P₂ sterically blocks T₂ from binding to the target antigen when the modified TCR is outside the tumor microenvironment.

Embodiment 50 provides the modified TCR of any one of embodiments 47-49, wherein P₂ is removed from the antigen binding site, and the antigen binding site of T₁ is exposed when the modified TCR is inside the tumor microenvironment.

Embodiment 51 provides the modified TCR of any one of embodiments 41-50, wherein P₂ comprises at least 70% sequence homology to the target antigen.

Embodiment 52 provides the modified TCR of any one of embodiments 41-51, wherein P₂ is a substrate for a tumor specific protease.

Embodiment 53 provides the modified TCR of any one of embodiments 41-52, wherein the tumor specific protease is selected from the group consisting of metalloprotease, serine protease, cysteine protease, threonine protease, and aspartic protease.

Embodiment 54 provides the modified TCR of any one of embodiments 41-53, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 55 provides the modified TCR of any one of embodiments 41-54, wherein P2 comprises a peptide sequence of at least 6 amino acids in length.

Embodiment 56 provides the modified TCR of any one of embodiments 41-55, wherein P2 comprises a peptide sequence of at least 10 amino acids in length.

Embodiment 57 provides the modified TCR of any one of embodiments 41-55, wherein P2 comprises a linear or cyclic peptide.

Embodiment 58 provides the modified TCR of any one of embodiments 41-57, wherein P2 comprises a modified amino acid, a non-natural amino acid, or a modified non-natural amino acids, or combination thereof.

Embodiment 59 provides the modified TCR of embodiment 58, wherein the modified amino acid or modified non-natural amino acid comprises a post-translational modification.

Embodiment 60 provides the modified TCR of any one of embodiments 41-59, wherein L2 is a peptide sequence having at least 5 to no more than 50 amino acids.

Embodiment 61 provides the modified TCR of any one of embodiments 41-60, wherein L2 has a formula selected from the group consisting of: (GS)n, wherein n is an integer from 6 to 20 (SEQ ID NO: 1); (G2S)n, wherein n is an integer from 4 to 13 (SEQ ID NO: 2); (G3S)n, wherein n is an integer from 3 to 10 (SEQ ID NO: 3); and (G4S)n, wherein n is an integer from 2 to 8 (SEQ ID NO: 4); and (G)n, wherein n is an integer from 12 to 40 (SEQ ID NO: 5).

Embodiment 62 provides the modified TCR of any one of embodiments 41-60, wherein L2 has a formula comprising (GGSGGD)n, wherein n is an integer from 2 to 6 (SEQ ID NO: 8).

Embodiment 63 provides the modified TCR of any one of embodiments 41-60, wherein L2 has a formula comprising (GGSGGE)n, wherein n is an integer from 2 to 6 (SEQ ID NO: 9).

Embodiment 64 provides the modified TCR of any one of embodiments 41-60, wherein L2 has a formula comprising (GGGSGSGGGGS)n, wherein n is an integer from 1 to 3 (SEQ ID NO: 6).

Embodiment 65 provides the modified TCR of any one of embodiments 41-60, wherein L2 has a formula comprising (GGGGGPGGGGP) n, wherein n is an integer from 1 to 3 (SEQ ID NO: 7).

Embodiment 66 provides the modified TCR of any one of embodiments 41-60, wherein L2 has a formula selected from: (GX)n, wherein X is serine, aspartic acid, glutamic acid, threonine, or proline and n is at least 20 (SEQ ID NO: 24); (GGX)n, wherein X is serine, aspartic acid, glutamic acid, threonine, or proline and n is at least 13 (SEQ ID NO: 25); (GGGX)n, wherein X is serine, aspartic acid, glutamic acid, threonine, or proline and n is at least 10 (SEQ ID NO: 26); (GGGGX)n, wherein X is serine, aspartic acid, glutamic acid, threonine, or proline and n is at least 8 (SEQ ID NO: 27); and (GzX)n, wherein X is serine, aspartic acid, glutamic acid, threonine, or proline and n is at least 15, and z is between 1 and 20 (SEQ ID NO: 28).

Embodiment 67 provides the modified TCR of any one of embodiments 41-60, wherein L2 is a substrate for a tumor specific protease.

Embodiment 68 provides the modified TCR of embodiment 67, wherein the tumor specific protease is selected from the group consisting of metalloprotease, serine protease, cysteine protease, threonine protease, and aspartic protease.

Embodiment 69 provides the modified TCR of embodiment 67, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 70 provides the modified TCR of any one of embodiments 41-60, wherein L2 comprises a plasmin cleavable amino acid sequence.

Embodiment 71 provides the modified TCR of embodiment 70, wherein the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15).

Embodiment 72 provides the modified TCR of any one of embodiments 41-60, wherein L2 comprises a Factor Xa cleavable amino acid sequence.

Embodiment 73 provides the modified TCR of embodiment 72, wherein the Factor Xa cleavable amino acid sequence is selected from the group consisting of IEGR (SEQ ID NO: 16), IDGR (SEQ ID NO: 17), and GGSIDGR (SEQ ID NO: 18).

Embodiment 74 provides the modified TCR of any one of embodiments 41-60, wherein L2 comprises an MMP cleavable amino acid sequence.

Embodiment 75 provides the modified TCR of embodiment 74, wherein the MMP cleavable amino acid sequence is PLGLWA (SEQ ID NO: 19).

Embodiment 76 provides the modified TCR of any one of embodiments 41-60, wherein L2 comprises a collagenase cleavable amino acid sequence.

Embodiment 77 provides the modified TCR of embodiment 76, wherein the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

Embodiment 78 provides the modified TCR of any one of embodiments 41-77, wherein L2 comprises a modified amino acid.

Embodiment 79 provides the modified TCR of embodiment 78, wherein the modified amino acid comprises a post-translational modification.

Embodiment 80 provides the modified TCR of any one of embodiments 41-79, wherein L2 comprises a non-natural amino acid or a modified non-natural amino acid, or combination thereof.

Embodiment 81 provides the modified TCR of embodiment 80, wherein the modified non-natural amino acid comprises a post-translational modification.

Embodiment 82 provides the modified TCR of any one of embodiments 1-81, wherein the TCR alpha extracellular domain, or fragment thereof, comprises three hyper-variable complementarity determining regions (CDRs).

Embodiment 83 provides the modified TCR of embodiment 82, wherein at least one CDR comprises a mutation to increase binding affinity or binding specificity to the target antigen or to increase binding affinity and binding specificity to the target antigen.

Embodiment 84 provides the modified TCR of any one of embodiments 1-83, wherein the TCR alpha extracellular domain, or fragment thereof, comprises a modified amino acid.

Embodiment 85 provides the modified TCR of embodiment 84, wherein the modified amino acid comprises a post-translational modification.

Embodiment 86 provides the modified TCR of any one of embodiments 1-85, wherein the TCR alpha extracellular domain, or fragment thereof, comprises a non-natural amino acid or a modified non-natural amino acid, or combination thereof.

Embodiment 87 provides the modified TCR of embodiment 86, wherein the modified non-natural amino acid comprises a post-translational modification.

Embodiment 88 provides the modified TCR of any one of embodiments 1-87, wherein the TCR beta extracellular domain, or fragment thereof, comprises three hyper-variable complementarity determining regions (CDRs).

Embodiment 89 provides the modified TCR of embodiment 88, wherein at least one CDR comprises a mutation to increase binding affinity or binding specificity to the target antigen or to increase binding affinity and binding specificity to the target antigen.

Embodiment 90 provides the modified TCR of any one of embodiments 1-89, wherein the TCR beta extracellular domain, or fragment thereof, comprises a modified amino acid.

Embodiment 91 provides the modified TCR of embodiment 90, wherein the modified amino acid comprises a post-translational modification.

Embodiment 92 provides the modified TCR of any one of embodiments 1-91, wherein the TCR beta extracellular domain, or fragment thereof, comprises a non-natural amino acid or a modified non-natural amino acid, or combination thereof.

Embodiment 93 provides the modified TCR of embodiment 92, wherein the modified non-natural amino acid comprises a post-translational modification.

Embodiment 94 provides a modified T cell receptor (TCR) comprising a polypeptide of formula III: T₃-L₃-P₃ (formula III) wherein: T₃ comprises either a TCR alpha extracellular domain, or a fragment thereof, or a TCR beta extracellular domain, or a fragment thereof, wherein T₃ binds to a target antigen, and the TCR alpha extracellular domain or fragment thereof and the TCR beta extracellular domain, or fragment thereof contain an antigen binding site; P₃ is a peptide that reduces binding of T₃ to the target antigen when the modified TCR is outside of a tumor microenvironment and that does not reduce binding of T₃ to the target antigen when the modified TCR is inside the tumor microenvironment, and L₃ is a linking moiety that connects T₃ to P₃ and L₃ is bound to T₃ at the N-terminus of T₃, wherein the modified TCR is a soluble TCR and is a functional TCR when inside the tumor microenvironment and is a nonfunctional TCR when outside the tumor microenvironment and P₃ or L₃ is a substrate for a tumor specific protease.

Embodiment 95 provides the modified TCR of embodiment 94, wherein P3 is bound to T3 through ionic interactions, electrostatic interactions, hydrophobic interactions, Pi-stacking interactions, and H-bonding interactions, or a combination thereof when the modified TCR is outside the tumor microenvironment.

Embodiment 96 provides the modified TCR of any one of embodiments 94-95, wherein P3 is bound to T3 at or near the antigen binding site when the modified TCR is outside the tumor microenvironment.

Embodiment 97 provides the modified TCR of any one of embodiments 94-96, wherein P3 inhibits the binding of T3 to the target antigen when the modified TCR is outside the tumor microenvironment, and P3 does not inhibit the binding of T3 to the target antigen when the modified TCR is inside the tumor microenvironment.

Embodiment 98 provides the modified TCR of any one of embodiments 94-97, wherein P3 sterically blocks T3 from binding to the target antigen when the modified TCR is outside the tumor microenvironment.

Embodiment 99 provides the modified TCR of any one of embodiments 96-98, wherein P3 is removed from the antigen binding site, and the antigen binding site of T3 is exposed when the modified TCR is inside the tumor microenvironment.

Embodiment 100 provides the modified TCR of any one of embodiments 94-99, wherein P3 comprises at least 70% sequence homology to the target antigen.

Embodiment 101 provides the modified TCR of any one of embodiments 94-100, wherein P3 is a substrate for a tumor specific protease.

Embodiment 102 provides the modified TCR of any one of embodiments 94-101, wherein the tumor specific protease is selected from the group consisting of metalloprotease, serine protease, cysteine protease, threonine protease, and aspartic protease.

Embodiment 103 provides the modified TCR of any one of embodiments 94-102, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 104 provides the modified TCR of any one of embodiments 94-103, wherein P3 comprises a peptide sequence of at least 6 amino acids in length.

Embodiment 105 provides the modified TCR of any one of embodiments 94-104, wherein P3 comprises a peptide sequence of at least 10 amino acids in length.

Embodiment 106 provides the modified TCR of any one of embodiments 94-104, wherein P3 comprises a linear or cyclic peptide.

Embodiment 107 provides the modified TCR of any one of embodiments 94-106, wherein P3 comprises a modified amino acid, a non-natural amino acid, or a modified non-natural amino acids, or combination thereof.

Embodiment 108 provides the modified TCR of embodiment 107, wherein the modified amino acid or modified non-natural amino acid comprises a post-translational modification.

Embodiment 109 provides the modified TCR of any one of embodiments 94-108, wherein L3 is a peptide sequence having at least 5 to no more than 50 amino acids.

Embodiment 110 provides the modified TCR of any one of embodiments 94-109, wherein L3 has a formula selected from the group consisting of: (GS)n, wherein n is an integer from 6 to 20 (SEQ ID NO: 1); (G2S)n, wherein n is an integer from 4 to 13 (SEQ ID NO: 2); (G3S)n, wherein n is an integer from 3 to 10 (SEQ ID NO: 3); and (G4S)n, wherein n is an integer from 2 to 8 (SEQ ID NO: 4); and (G)n, wherein n is an integer from 12 to 40 (SEQ ID NO: 5).

Embodiment 111 provides the modified TCR of any one of embodiments 94-109, wherein L3 has a formula comprising (GGSGGD)n, wherein n is an integer from 2 to 6 (SEQ ID NO: 8).

Embodiment 112 provides the modified TCR of any one of embodiments 94-109, wherein L3 has a formula comprising (GGSGGE)n, wherein n is an integer from 2 to 6 (SEQ ID NO: 9).

Embodiment 113 provides the modified TCR of any one of embodiments 94-109, wherein L3 has a formula comprising (GGGSGSGGGGS)n, wherein n is an integer from 1 to 3 (SEQ ID NO: 6).

Embodiment 114 provides the modified TCR of any one of embodiments 94-109, wherein L3 has a formula comprising (GGGGGPGGGGP) n, wherein n is an integer from 1 to 3 (SEQ ID NO: 7).

Embodiment 115 provides the modified TCR of any one of embodiments 94-109, wherein L3 has a formula selected from: (GX)n, wherein X is serine, aspartic acid, glutamic acid, threonine, or proline and n is at least 20 (SEQ ID NO: 24); (GGX)n, wherein X is serine, aspartic acid, glutamic acid, threonine, or proline and n is at least 13 (SEQ ID NO: 25); (GGGX)n, wherein X is serine, aspartic acid, glutamic acid, threonine, or proline and n is at least 10 (SEQ ID NO: 26); (GGGGX)n, wherein X is serine, aspartic acid, glutamic acid, threonine, or proline and n is at least 8 (SEQ ID NO: 27); and (GzX)n, wherein X is serine, aspartic acid, glutamic acid, threonine, or proline and n is at least 15, and z is between 1 and 20 (SEQ ID NO: 28).

Embodiment 116 provides the modified TCR of any one of embodiments 94-109, wherein L3 is a substrate for a tumor specific protease.

Embodiment 117 provides the modified TCR of embodiment 116, wherein the tumor specific protease is selected from the group consisting of metalloprotease, serine protease, cysteine protease, threonine protease, and aspartic protease.

Embodiment 118 provides the modified TCR of embodiment 116, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 119 provides the modified TCR of any one of embodiments 94-109, wherein L3 comprises a plasmin cleavable amino acid sequence.

Embodiment 120 provides the modified TCR of embodiment 26, wherein the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15).

Embodiment 121 provides the modified TCR of any one of claims 94-109, wherein L3 comprises a Factor Xa cleavable amino acid sequence.

Embodiment 122 provides the modified TCR of embodiment 28, wherein the Factor Xa cleavable amino acid sequence is selected from the group consisting of IEGR (SEQ ID NO: 16), IDGR (SEQ ID NO: 17), and GGSIDGR (SEQ ID NO: 18).

Embodiment 123 provides the modified TCR of any one of embodiments 94-109, wherein L3 comprises an MMP cleavable amino acid sequence.

Embodiment 124 provides the modified TCR of claim 123, wherein the MMP cleavable amino acid sequence is PLGLWA (SEQ ID NO: 19).

Embodiment 125 provides the modified TCR of any one of embodiments 94-109, wherein L3 comprises a collagenase cleavable amino acid sequence.

Embodiment 126 provides the modified TCR of embodiment 125, wherein the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

Embodiment 127 provides the modified TCR of any one of embodiments 94-126, wherein L3 comprises a modified amino acid.

Embodiment 128 provides the modified TCR of embodiment 127, wherein the modified amino acid comprises a post-translational modification.

Embodiment 129 provides the modified TCR of any one of embodiments 94-128, wherein L3 comprises a non-natural amino acid or a modified non-natural amino acid, or combination thereof.

Embodiment 130 provides the modified TCR of embodiment 129, wherein the modified non-natural amino acid comprises a post-translational modification.

Embodiment 131 provides the modified TCR of any one of embodiments 94-130, wherein the target antigen is selected from the group consisting of MAGE-A3, NY-ESO-1, gp100, WT1, and tyrosinase.

Embodiment 132 provides the modified TCR of any one of embodiments 94-133, wherein T3 comprises the TCR alpha extracellular domain, or fragment thereof, and the modified TCR further comprises a second polypeptide comprising a TCR beta extracellular domain, or a fragment thereof wherein the TCR beta extracellular domain or fragment thereof contains an antigen binding site.

Embodiment 133 provides the modified TCR of any one of embodiments 94-133, wherein T3 comprises the TCR beta extracellular domain, or fragment thereof, and the modified TCR further comprises a second polypeptide comprising a TCR alpha extracellular domain, or a fragment thereof wherein the TCR alpha extracellular domain or fragment thereof contains an antigen binding site.

Embodiment 134 provides the modified TCR of any one of embodiments 94-133, wherein T₃ comprises the TCR alpha extracellular domain, or fragment thereof, and the modified TCR further comprises a second polypeptide of formula IV: T₄-L₄-P₄ (formula IV) wherein T₄ comprises a TCR beta extracellular domain, or fragment thereof, wherein T₄ binds to the target antigen and the TCR beta extracellular domain or fragment thereof contains an antigen binding site; P₄ is a peptide that reduces binding of T₄ to the target antigen when the modified TCR is outside of a tumor microenvironment and that does not reduce binding of T₄ to the target antigen when the modified TCR is inside the tumor microenvironment, and L₄ is a linking moiety that connects T₄ to P₄ and L₄ is bound to T₄ at the N-terminus of T₄, wherein P₄ or L₄ is a substrate for a tumor specific protease.

Embodiment 135 provides the modified TCR of any one of embodiments 132-134, wherein the TCR alpha extracellular domain, or fragment thereof, and the TCR beta extracellular domain, or fragment thereof, are connected by a disulfide bond.

Embodiment 136 provides the modified TCR of any one of embodiments 134-135, wherein the TCR alpha extracellular domain, or fragment thereof, comprises an alpha chain TRAC constant domain sequence and the TCR beta extracellular domain, or fragment thereof, comprises a beta chain TRBC1 or TRBC2 constant domain sequence.

Embodiment 137 provides the modified TCR of any one of embodiments 134-136, wherein Cys4 of the alpha chain TRAC constant domain sequence is modified by truncation or substitution and Cys2 of exon 2 of the beta chain TRBC1 or TRBC2 constant domain sequence is modified by truncation or substitution, thereby deleting a native disulfide bond.

Embodiment 138 provides the modified TCR of any one of embodiments 134-137, wherein Thr48 of the alpha chain TRAC constant domain sequence is mutated to Cys and Ser57 of the beta chain TRBC1 or TRBC2 constant domain sequence is mutated to Cys.

Embodiment 139 provides the modified TCR of any one of embodiments 134-138, wherein P4 is bound to T4 through ionic interactions, electrostatic interactions, hydrophobic interactions, Pi-stacking interactions, and H-bonding interactions, or a combination thereof when the modified TCR is outside the tumor microenvironment.

Embodiment 140 provides the modified TCR of any one of embodiments 134-139, wherein P4 is bound to T4 at or near the antigen binding site when the modified TCR is outside the tumor microenvironment.

Embodiment 141 provides the modified TCR of any one of embodiments 134-140, wherein P4 inhibits the binding of T4 to the target antigen when the modified TCR is outside the tumor microenvironment, and P4 does not inhibit the binding of T4 to the target antigen when the modified TCR is inside the tumor microenvironment.

Embodiment 142 provides the modified TCR of any one of embodiments 134-141, wherein P4 sterically blocks T4 from binding to the target antigen when the modified TCR is outside the tumor microenvironment.

Embodiment 143 provides the modified TCR of any one of embodiments 134-142, wherein P4 is removed from the antigen binding site, and the antigen binding site of T4 is exposed when the modified TCR is inside the tumor microenvironment.

Embodiment 144 provides the modified TCR of any one of embodiments 134-143, wherein P4 comprises at least 70% sequence homology to the target antigen.

Embodiment 145 provides the modified TCR of any one of embodiments 134-144, wherein P4 is a substrate for a tumor specific protease.

Embodiment 146 provides the modified TCR of any one of embodiments 134-145, wherein the tumor specific protease is selected from the group consisting of metalloprotease, serine protease, cysteine protease, threonine protease, and aspartic protease.

Embodiment 147 provides the modified TCR of any one of embodiments 134-146, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 148 provides the modified TCR of any one of embodiments 134-147, wherein P4 comprises a peptide sequence of at least 6 amino acids in length.

Embodiment 149 provides the modified TCR of any one of embodiments 134-148, wherein P4 comprises a peptide sequence of at least 10 amino acids in length.

Embodiment 150 provides the modified TCR of any one of embodiments 134-148, wherein P4 comprises a linear or cyclic peptide.

Embodiment 151 provides the modified TCR of any one of embodiments 134-150, wherein P4 comprises a modified amino acid, a non-natural amino acid, or a modified non-natural amino acids, or combination thereof.

Embodiment 152 provides the modified TCR of embodiment 151, wherein the modified amino acid or modified non-natural amino acid comprises a post-translational modification.

Embodiment 153 provides the modified TCR of any one of embodiments 134-152, wherein L4 is a peptide sequence having at least 5 to no more than 50 amino acids.

Embodiment 154 provides the modified TCR of any one of embodiments 134-153, wherein L4 has a formula selected from the group consisting of: (GS)n, wherein n is an integer from 6 to 20 (SEQ ID NO: 1); (G2S)n, wherein n is an integer from 4 to 13 (SEQ ID NO: 2); (G3S)n, wherein n is an integer from 3 to 10 (SEQ ID NO: 3); and (G4S)n, wherein n is an integer from 2 to 8 (SEQ ID NO: 4); and (G)n, wherein n is an integer from 12 to 40 (SEQ ID NO: 5).

Embodiment 155 provides the modified TCR of any one of embodiments 134-153, wherein L4 has a formula comprising (GGSGGD)n, wherein n is an integer from 2 to 6 (SEQ ID NO: 8).

Embodiment 156 provides the modified TCR of any one of embodiments 134-153, wherein L4 has a formula comprising (GGSGGE)n, wherein n is an integer from 2 to 6 (SEQ ID NO: 9).

Embodiment 157 provides the modified TCR of any one of embodiments 134-153, wherein L4 has a formula comprising (GGGSGSGGGGS)n, wherein n is an integer from 1 to 3 (SEQ ID NO: 6).

Embodiment 158 provides the modified TCR of any one of embodiments 134-153, wherein L4 has a formula comprising (GGGGGPGGGGP) n, wherein n is an integer from 1 to 3 (SEQ ID NO: 7).

Embodiment 159 provides the modified TCR of any one of embodiments 134-153, wherein L4 has a formula selected from: (GX)n, wherein X is serine, aspartic acid, glutamic acid, threonine, or proline and n is at least 20 (SEQ ID NO: 24); (GGX)n, wherein X is serine, aspartic acid, glutamic acid, threonine, or proline and n is at least 13 (SEQ ID NO: 25); (GGGX)n, wherein X is serine, aspartic acid, glutamic acid, threonine, or proline and n is at least 10 (SEQ ID NO: 26); (GGGGX)n, wherein X is serine, aspartic acid, glutamic acid, threonine, or proline and n is at least 8 (SEQ ID NO: 27); and (GzX)n, wherein X is serine, aspartic acid, glutamic acid, threonine, or proline and n is at least 15, and z is between 1 and 20 (SEQ ID NO: 28).

Embodiment 160 provides the modified TCR of any one of embodiments 134-153, wherein L4 is a substrate for a tumor specific protease.

Embodiment 161 provides the modified TCR of embodiment 160, wherein the tumor specific protease is selected from the group consisting of metalloprotease, serine protease, cysteine protease, threonine protease, and aspartic protease.

Embodiment 162 provides the modified TCR of embodiment 161, wherein the tumor specific protease is selected from the group consisting of: ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 163 provides the modified TCR of any one of embodiments 134-153, wherein L4 comprises a plasmin cleavable amino acid sequence.

Embodiment 164 provides the modified TCR of embodiment 163, wherein the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15).

Embodiment 165 provides the modified TCR of any one of embodiments 134-153, wherein L4 comprises a Factor Xa cleavable amino acid sequence.

Embodiment 166 provides the modified TCR of embodiment 165, wherein the Factor Xa cleavable amino acid sequence is selected from the group consisting of IEGR (SEQ ID NO: 16), IDGR (SEQ ID NO: 17), and GGSIDGR (SEQ ID NO: 18).

Embodiment 167 provides the modified TCR of any one of embodiments 134-153, wherein L4 comprises an MMP cleavable amino acid sequence.

Embodiment 168 provides the modified TCR of embodiment 167, wherein the MMP cleavable amino acid sequence is PLGLWA (SEQ ID NO: 19).

Embodiment 169 provides the modified TCR of any one of embodiments 134-153, wherein L4 comprises a collagenase cleavable amino acid sequence.

Embodiment 170 provides the modified TCR of embodiment 169, wherein the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

Embodiment 171 provides the modified TCR of any one of embodiments 134-170, wherein L4 comprises a modified amino acid.

Embodiment 172 provides the modified TCR of embodiment 171, wherein the modified amino acid comprises a post-translational modification.

Embodiment 173 provides the modified TCR of any one of embodiments 134-172, wherein L4 comprises a non-natural amino acid or a modified non-natural amino acid, or combination thereof.

Embodiment 174 provides the modified TCR of embodiment 173, wherein the modified non-natural amino acid comprises a post-translational modification.

Embodiment 175 provides the modified TCR of any one of embodiments 88-162, wherein the TCR alpha extracellular domain, or fragment thereof, comprises three hyper-variable complementarity determining regions (CDRs).

Embodiment 176 provides the modified TCR of embodiment 163, wherein at least one CDR comprises a mutation to increase binding affinity or binding specificity to the target antigen or to increase binding affinity and binding specificity to the target antigen.

Embodiment 177 provides the modified TCR of any one of embodiments 94-176, wherein the TCR alpha extracellular domain, or fragment thereof, comprises a truncated transmembrane domain.

Embodiment 179 provides the modified TCR of any one of embodiments 94-177, wherein the TCR alpha extracellular domain, or fragment thereof, comprises a modified amino acid.

Embodiment 180 provides the modified TCR of embodiment 179, wherein the modified amino acid comprises a post-translational modification.

Embodiment 181 provides the modified TCR of any one of embodiments 94-180, wherein the TCR alpha extracellular domain, or fragment thereof, comprises a non-natural amino acid or a modified non-natural amino acid, or combination thereof.

Embodiment 182 provides the modified TCR of embodiment 181, wherein the modified non-natural amino acid comprises a post-translational modification.

Embodiment 183 provides the modified TCR of any one of embodiments 94-182, wherein the TCR beta extracellular domain, or fragment thereof, comprises three hyper-variable complementarity determining regions (CDRs).

Embodiment 184 provides the modified TCR of embodiment 94-183, wherein at least one CDR comprises a mutation to increase binding affinity or binding specificity to the target antigen or to increase binding affinity and binding specificity to the target antigen.

Embodiment 185 provides the modified TCR of any one of embodiments 94-184, wherein the TCR beta extracellular domain, or fragment thereof, comprises a truncated transmembrane domain.

Embodiment 187 provides the modified TCR of any one of embodiments 94-186, wherein the TCR beta extracellular domain, or fragment thereof, comprises a modified amino acid.

Embodiment 188 provides the modified TCR of embodiment 187, wherein the modified amino acid comprises a post-translational modification.

Embodiment 189 provides the modified TCR of any one of embodiments 94-188, wherein the TCR beta extracellular domain, or fragment thereof, comprises a non-natural amino acid or a modified non-natural amino acid, or combination thereof.

Embodiment 190 provides the modified TCR of embodiment 189, wherein the modified non-natural amino acid comprises a post-translational modification.

Embodiment 191 provides a modified T cell receptor (TCR) comprising a polypeptide of formula V: T₅-L₅-P₅ (formula V) wherein T₅ comprises a variable region of a TCR alpha extracellular domain, or fragment thereof, and a variable region of a TCR beta extracellular domain, or fragment thereof, wherein T₅ binds to a target antigen and the variable region of TCR alpha extracellular domain, or fragment thereof, and the variable region of the TCR beta extracellular domain, or fragment thereof contain an antigen binding site, P₅ is a peptide that reduces binding of T₅ to the target antigen when the modified TCR is outside of a tumor microenvironment and that does not reduce binding of T₅ to the target antigen when the modified TCR is inside the tumor microenvironment, and L₅ is a linking moiety that connects T₅ to P₅ and L₅ is bound to T₅ at the N-terminus of T₅, wherein the modified TCR is a soluble TCR and is a functional TCR when inside the tumor microenvironment and is a nonfunctional TCR when outside the tumor microenvironment and P₅ or L₅ is a substrate for a tumor specific protease.

Embodiment 192 provides the modified TCR of embodiment 191, wherein P5 is bound to T5 through ionic interactions, electrostatic interactions, hydrophobic interactions, Pi-stacking interactions, and H-bonding interactions, or a combination thereof when the modified TCR is outside the tumor microenvironment.

Embodiment 193 provides the modified TCR of any one of embodiments 191-192, wherein P5 is bound to T5 at or near the antigen binding site when the modified TCR is outside the tumor microenvironment.

Embodiment 194 provides the modified TCR of any one of embodiments 191-193, wherein P5 inhibits the binding of T3 to the target antigen when the modified TCR is outside the tumor microenvironment, and P3 does not inhibit the binding of T5 to the target antigen when the modified TCR is inside the tumor microenvironment.

Embodiment 195 provides the modified TCR of any one of embodiments 191-194, wherein P5 sterically blocks T3 from binding to the target antigen when the modified TCR is outside the tumor microenvironment.

Embodiment 196 provides the modified TCR of any one of embodiments 191-195, wherein P5 is removed from the antigen binding site, and the antigen binding site of T5 is exposed when the modified TCR is inside the tumor microenvironment.

Embodiment 197 provides the modified TCR of any one of embodiments 191-196, wherein P5 comprises at least 70% sequence homology to the target antigen.

Embodiment 198 provides the modified TCR of any one of embodiments 191-197, wherein P5 is a substrate for a tumor specific protease.

Embodiment 199 provides the modified TCR of any one of embodiments 191-198, wherein the tumor specific protease is selected from the group consisting of metalloprotease, serine protease, cysteine protease, threonine protease, and aspartic protease.

Embodiment 200 provides the modified TCR of any one of embodiments 191-198, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 201 provides the modified TCR of any one of embodiments 191-200, wherein P5 comprises a peptide sequence of at least 6 amino acids in length.

Embodiment 202 provides the modified TCR of any one of embodiments 191-201, wherein P5 comprises a peptide sequence of at least 10 amino acids in length.

Embodiment 203 provides the modified TCR of any one of embodiments 191-201, wherein P5 comprises a linear or cyclic peptide.

Embodiment 204 provides the modified TCR of any one of embodiments 191-203, wherein P5 comprises a modified amino acid, a non-natural amino acid, or a modified non-natural amino acids, or combination thereof.

Embodiment 205 provides the modified TCR of embodiment 204, wherein the modified amino acid or modified non-natural amino acid comprises a post-translational modification.

Embodiment 206 provides the modified TCR of any one of embodiments 191-205, wherein L5 is a peptide sequence having at least 5 to no more than 50 amino acids.

Embodiment 207 provides the modified TCR of any one of embodiments 191-206, wherein L5 has a formula selected from the group consisting of: (GS)n, wherein n is an integer from 6 to 20 (SEQ ID NO: 1); (G2S)n, wherein n is an integer from 4 to 13 (SEQ ID NO: 2); (G3S)n, wherein n is an integer from 3 to 10 (SEQ ID NO: 3); and (G4S)n, wherein n is an integer from 2 to 8 (SEQ ID NO: 4); and (G)n, wherein n is an integer from 12 to 40 (SEQ ID NO: 5).

Embodiment 208 provides the modified TCR of any one of embodiments 191-206, wherein L5 has a formula comprising (GGSGGD)n, wherein n is an integer from 2 to 6 (SEQ ID NO: 8).

Embodiment 209 provides the modified TCR of any one of embodiments 191-206, wherein L5 has a formula comprising (GGSGGE)n, wherein n is an integer from 2 to 6 (SEQ ID NO: 9).

Embodiment 210 provides the modified TCR of any one of embodiments 191-206, wherein L5 has a formula comprising (GGGSGSGGGGS)n, wherein n is an integer from 1 to 3 (SEQ ID NO: 6).

Embodiment 211 provides the modified TCR of any one of embodiments 191-206, wherein L5 has a formula comprising (GGGGGPGGGGP) n, wherein n is an integer from 1 to 3 (SEQ ID NO: 7).

Embodiment 212 provides the modified TCR of any one of embodiments 191-206, wherein L5 has a formula selected from: (GX)n, wherein X is serine, aspartic acid, glutamic acid, threonine, or proline and n is at least 20 (SEQ ID NO: 24); (GGX)n, wherein X is serine, aspartic acid, glutamic acid, threonine, or proline and n is at least 13 (SEQ ID NO: 25); (GGGX)n, wherein X is serine, aspartic acid, glutamic acid, threonine, or proline and n is at least 10 (SEQ ID NO: 26); (GGGGX)n, wherein X is serine, aspartic acid, glutamic acid, threonine, or proline and n is at least 8 (SEQ ID NO: 27); and (GzX)n, wherein X is serine, aspartic acid, glutamic acid, threonine, or proline and n is at least 15, and z is between 1 and 20 (SEQ ID NO: 28).

Embodiment 213 provides the modified TCR of any one of embodiments 191-206, wherein L5 is a substrate for a tumor specific protease.

Embodiment 214 provides the modified TCR of embodiment 213, wherein the tumor specific protease is selected from the group consisting of metalloprotease, serine protease, cysteine protease, threonine protease, and aspartic protease.

Embodiment 215 provides the modified TCR of embodiment 213, wherein the tumor specific protease is selected from the group consisting of ADAM10, ADAM12, ADAM17, ADAMTS, ADAMTS5, BACE, Caspase 1, Caspase 2, Caspase 3, Caspase 4, Caspase 5, Caspase 6, Caspase 7, tPA, Caspase 8, Caspase 9, Caspase 10, Caspase 11, Caspase 12, Caspase 13, Caspase 14, Cathepsin A, Cathepsin B, Cathepsin D, Cathepsin E, Cathepsin K, MT1-MMP, HCV-NS3/4A, Cathepsin S, FAP, Granzyme B, Guanidinobenzoatase, Hepsin, Human Neutrophil Elastase, Legumain, Matriptase 2, Meprin, MMP 1, MMP 2, MMP 3, MMP 7, neurosin, MMP 8, MMP 9, MMP 13, MMP 14, MT-SP1, Neprilysin, HCV-1/153/4, Plasmin, PSA, PSMA, TACE, TMPRSS 3/4, uPA, and Calpain.

Embodiment 216 provides the modified TCR of any one of embodiments 191-215, wherein L5 comprises a plasmin cleavable amino acid sequence.

Embodiment 217 provides the modified TCR of embodiment 216, wherein the plasmin cleavable amino acid sequence is selected from the group consisting of PRFKIIGG (SEQ ID NO: 10), PRFRIIGG (SEQ ID NO: 11), SSRHRRALD (SEQ ID NO: 12), RKSSIIIRMRDVVL (SEQ ID NO: 13), SSSFDKGKYKKGDDA (SEQ ID NO: 14), and SSSFDKGKYKRGDDA (SEQ ID NO: 15).

Embodiment 218 provides the modified TCR of any one of embodiments 191-215, wherein L5 comprises a Factor Xa cleavable amino acid sequence.

Embodiment 219 provides the modified TCR of embodiment 218, wherein the Factor Xa cleavable amino acid sequence is selected from the group consisting of IEGR (SEQ ID NO: 16), IDGR (SEQ ID NO: 17), and GGSIDGR (SEQ ID NO: 18).

Embodiment 220 provides the modified TCR of any one of embodiments 191-215, wherein L5 comprises an MMP cleavable amino acid sequence.

Embodiment 221 provides the modified TCR of embodiment 220, wherein the MMP cleavable amino acid sequence is PLGLWA (SEQ ID NO: 19).

Embodiment 222 provides the modified TCR of any one of embodiments 191-215, wherein L5 comprises a collagenase cleavable amino acid sequence.

Embodiment 223 provides the modified TCR of embodiment 222, wherein the collagenase cleavable amino acid sequence is selected from the group consisting of GPQGIAGQ (SEQ ID NO: 20), GPQGLLGA (SEQ ID NO: 21), GIAGQ (SEQ ID NO: 22), GPLGIAGI (SEQ ID NO: 23), GPEGLRVG (SEQ ID NO: 29), YGAGLGVV (SEQ ID NO: 30), AGLGVVER (SEQ ID NO: 31), AGLGISST (SEQ ID NO: 32), EPQALAMS (SEQ ID NO: 33), QALAMSAI (SEQ ID NO: 34), AAYHLVSQ (SEQ ID NO: 35), MDAFLESS (SEQ ID NO: 36), ESLPVVAV (SEQ ID NO: 37), SAPAVESE (SEQ ID NO: 38), and DVAQFVLT (SEQ ID NO: 39).

Embodiment 224 provides the modified TCR of any one of embodiments 94-223, wherein L5 comprises a modified amino acid.

Embodiment 225 provides the modified TCR of embodiments 224, wherein the modified amino acid comprises a post-translational modification.

Embodiment 226 provides the modified TCR of any one of embodiments 191-226, wherein L5 comprises a non-natural amino acid or a modified non-natural amino acid, or combination thereof.

Embodiment 227 provides the modified TCR of embodiment 226, wherein the modified non-natural amino acid comprises a post-translational modification.

Embodiment 228 provides the modified TCR of any one of embodiments 191-227, wherein the target antigen is from a gene family selected from the group consisting of: is selected from the group consisting of MAGE-A3, NY-ESO-1, gp100, WT1, and tyrosinase.

Embodiment 229 provides the modified TCR of any one of embodiments 191-228, wherein T₅ comprises a formula: Vα-L₅₁-Vβ wherein Vα is the variable region of the TCR alpha extracellular domain, or fragment thereof, Vβ is the variable region of the TCR beta extracellular domain, or fragment thereof, and L₅₁ is a sequence that connects Vα and Vβ, wherein Vα is N-terminal to L₅₁.

Embodiment 230 provides the modified TCR of any one of embodiments 191-228, wherein T5 comprises a formula: Vβ-L52-Vα wherein Vβ is the variable region of the TCR beta extracellular domain, or fragment thereof, Vα is the variable region of the TCR alpha extracellular domain, or fragment thereof, and L52 is a sequence that connects Vβ and Vα, wherein Vβ is N-terminal to L52.

Embodiment 231 provides the modified TCR of any one of embodiments 191-228, wherein T5 comprises a formula: Vα-L53-Vβ-Cβ wherein Vα is the variable region of the TCR alpha extracellular domain, or fragment thereof, Vβ is the variable region of the TCR beta extracellular domain, or fragment thereof, Cβ is a constant region of the TCR beta extracellular domain, or fragment thereof, and L53 is a sequence that connects Vα and Vβ, wherein Vα is N-terminal to L53.

Embodiment 232 provides the modified TCR of any one of embodiments 191-228, wherein T5 comprises a formula: Vβ-Cβ-L54-Vα wherein Vβ is the variable region of the TCR beta extracellular domain, or fragment thereof, Cβ is a constant region of the TCR beta extracellular domain, or fragment thereof, Vα is the variable region of the TCR alpha extracellular domain, or fragment thereof, and L54 is a sequence that connects Cβ and Vα, wherein Vβ is N-terminal to L54.

Embodiment 233 provides the modified TCR of any one of embodiments 191-228, wherein T5 comprises a formula: Vα-Cα-L55-Vβ wherein Vα is the variable region of the TCR alpha extracellular domain, or fragment thereof, Cα is a constant region of the TCR alpha extracellular domain, or fragment thereof, Vβ is the variable region of the TCR beta extracellular domain, or fragment thereof, and L55 is a sequence that connects Cα and Vβ, wherein Vα is N-terminal to L55.

Embodiment 234 provides the modified TCR of any one of embodiments 191-228, wherein T5 comprises a formula: Vβ-L56-Vα-Cα wherein Vβ is the variable region of the TCR beta extracellular domain, or fragment thereof, Vα is the variable region of the TCR alpha extracellular domain, or fragment thereof, Cα is a constant region of the TCR alpha extracellular domain, or fragment thereof, and L56 is a sequence that connects Vβ and Vα, wherein Vβ is N-terminal to L56.

Embodiment 235 provides the modified TCR of any one of embodiments 191-234, wherein the TCR alpha extracellular domain, or fragment thereof, comprises three hyper-variable complementarity determining regions (CDRs).

Embodiment 236 provides the modified TCR of embodiment 235, wherein at least one CDR comprises a mutation to increase binding affinity or binding specificity to the target antigen or to increase binding affinity and binding specificity to the target antigen.

Embodiment 237 provides the modified TCR of any one of embodiments 191-236, wherein the variable region of the TCR alpha extracellular domain, or fragment thereof, comprises a modified amino acid.

Embodiment 238 provides the modified TCR of embodiment 237, wherein the modified amino acid comprises a post-translational modification.

Embodiment 239 provides the modified TCR of any one of embodiments 191-238, wherein the variable region of the TCR alpha extracellular domain, or fragment thereof, comprises a non-natural amino acid or a modified non-natural amino acid, or combination thereof.

Embodiment 240 provides the modified TCR of embodiment 239, wherein the modified non-natural amino acid comprises a post-translational modification.

Embodiment 241 provides the modified TCR of any one of embodiments 191-231, wherein the variable region of the TCR beta extracellular domain, or fragment thereof, comprises three hyper-variable complementarity determining regions (CDRs).

Embodiment 242 provides the modified TCR of embodiment 232, wherein at least one CDR comprises a mutation to increase binding affinity or binding specificity to the target antigen or to increase binding affinity and binding specificity to the target antigen.

Embodiment 243 provides the modified TCR of any one of embodiments 191-233, wherein the variable region of the TCR beta extracellular domain, or fragment thereof, comprises a modified amino acid.

Embodiment 244 provides the modified TCR of embodiment 84, wherein the modified amino acid comprises a post-translational modification.

Embodiment 245 provides the modified TCR of any one of embodiments 191-235, wherein the variable region of the TCR beta extracellular domain, or fragment thereof, comprises a non-natural amino acid or a modified non-natural amino acid, or combination thereof.

Embodiment 246 provides the modified TCR of embodiment 236, wherein the modified non-natural amino acid comprises a post-translational modification.

Embodiment 247 provides the modified TCR of any one of embodiments 191-237, wherein T5 further comprises a truncated transmembrane domain.

Embodiment 249 provides the modified TCR of any one of embodiments 1-247, wherein the TCR further comprises a detectable label, a therapeutic agent, or a pharmacokinetic modifying moiety.

Embodiment 249 provides the modified TCR of any one of embodiments 1-39, 41-93, wherein T₁ is a full length TCR alpha chain polypeptide.

Embodiment 252 provides the modified TCR of any one of embodiments 41-93, wherein T2 is a full length TCR beta chain polypeptide.

Embodiment 253 provides the modified TCR of any one of embodiments 1-38, and 40, wherein T1 is a full length TCR beta chain polypeptide.

Embodiment 254 provides an isolated or non-naturally occurring cell, presenting a modified TCR according to any one of claims 1-253.

Embodiment 255 provides the isolated or non-naturally occurring cell according to embodiment 254, wherein the isolated or non-naturally occurring cell is a T cell.

Embodiment 256 provides a pharmaceutical composition, comprising: the isolated or non-naturally occurring cells according to embodiments 254 and 255; and a pharmaceutically acceptable excipient.

Embodiment 257 provides a pharmaceutical composition, comprising: the modified TCR according to embodiments 94-253; and a pharmaceutically acceptable excipient.

Embodiment 258 provides an isolated recombinant nucleic acid molecule encoding a polypeptide comprising a formula I: T1-L1-P1 (formula I) wherein: T1 comprises a transmembrane domain and either a TCR alpha extracellular domain, or fragment thereof, or a TCR beta extracellular domain, or fragment thereof, wherein T1 binds to a target antigen and the TCR alpha extracellular domain or fragment thereof and the TCR beta extracellular domain, or fragment thereof contain an antigen binding site, P1 is a peptide that reduces binding of T1 to the target antigen when the modified TCR is outside of a tumor microenvironment and that does not reduce binding of T1 to the target antigen when the modified TCR is inside the tumor microenvironment, and L1 is a linking moiety that connects T1 to P1 and L1 is bound to T1 at the N-terminus of T1, wherein the modified TCR is a functional TCR when inside the tumor microenvironment and is a nonfunctional TCR when outside the tumor microenvironment and P1 or L1 is a substrate for a tumor specific protease.

Embodiment 259 provides an isolated recombinant nucleic acid molecule encoding a polypeptide comprising a formula III: T3-L3-P3 (formula III) wherein: T3 comprises either a TCR alpha extracellular domain, or fragment thereof, or a TCR beta extracellular domain, or fragment thereof, wherein T3 binds to a target antigen and the TCR alpha extracellular domain or fragment thereof and the TCR beta extracellular domain, or fragment thereof contain an antigen binding site, P3 is a peptide that reduces binding of T3 to the target antigen when the modified TCR is outside of a tumor microenvironment and that does not reduce binding of T3 to the target antigen when the modified TCR is inside the tumor microenvironment, and L3 is a linking moiety that connects T3 to P3 and L3 is bound to T3 at the N-terminus of T3, wherein the modified TCR is a soluble TCR and is a functional TCR when inside the tumor microenvironment and is a nonfunctional TCR when outside the tumor microenvironment and P3 or L3 is a substrate for a tumor specific protease.

Embodiment 260 provides an isolated recombinant nucleic acid molecule encoding a polypeptide comprising a formula V: T5-L5-P5 (formula V) wherein T5 comprises a variable region of a TCR alpha extracellular domain, or fragment thereof, and a variable region of a TCR beta extracellular domain, or fragment thereof, wherein T5 binds to a target antigen and the variable region of TCR alpha extracellular domain, or fragment thereof, and the variable region of the TCR beta extracellular domain, or fragment thereof contain an antigen binding site, P5 is a peptide that reduces binding of T5 to the target antigen when the modified TCR is outside of a tumor microenvironment and that does not reduce binding of T5 to the target antigen when the modified TCR is inside the tumor microenvironment, and L5 is a linking moiety that connects T5 to P5 and L5 is bound to T5 at the N-terminus of T5, wherein the modified TCR is a soluble TCR and is a functional TCR when inside the tumor microenvironment and is a nonfunctional TCR when outside the tumor microenvironment and P5 or L5 is a substrate for a tumor specific protease.

Embodiment 261 provides a vector comprising a nucleic acid molecule encoding a modified TCR of any one of embodiments 258-260.

Embodiment 262 provides the modified TCR of any one of embodiments 94-185, wherein the modified TCR further comprises an effector domain.

Embodiment 263 provides the modified TCR of any one of embodiments 1-6, wherein P₁ comprises less than 70% sequence homology to the target antigen.

Embodiment 264 provides the modified TCR of any one of embodiments 41-50, wherein P₂ comprises less than 70% sequence homology to the target antigen.

Embodiment 265 provides the modified TCR of any one of embodiments 94-99, wherein P3 comprises less than 70% sequence homology to the target antigen.

Embodiment 266 provides the modified TCR of any one of embodiments 134-143, wherein P4 comprises less than 70% sequence homology to the target antigen.

Embodiment 267 provides the modified TCR of any one of embodiments 191-196, wherein P5 comprises less than 70% sequence homology to the target antigen.

What is claimed is:

1. A modified T cell receptor (TCR) comprising a polypeptide of formula III:
T₃-L₃-P₃ (formula III)
wherein:
T₃ comprises either a TCR alpha extracellular domain, or a fragment thereof, or a TCR beta extracellular domain, or a fragment thereof, wherein T₃ binds to a target antigen, and the TCR alpha extracellular domain or fragment thereof and the TCR beta extracellular domain, or fragment thereof contain an antigen binding site;
P₃ is a peptide that reduces binding of T₃ to the target antigen when the modified TCR is outside of a tumor microenvironment and that does not reduce binding of T₃ to the target antigen when the modified TCR is inside the tumor microenvironment; and
L₃ is a linking moiety that connects T₃ to P₃ and L₃ is bound to T₃ at the N-terminus of T₃,
wherein the modified TCR is a soluble TCR and is a functional TCR when inside the tumor microenvironment and is a nonfunctional TCR when outside the tumor microenvironment and P₃ or L₃ is a substrate for a tumor specific protease.

2. The modified TCR of claim 1, wherein P₃ is bound to T₃ through ionic interactions, electrostatic interactions, hydrophobic interactions, Pi-stacking interactions, and H-bonding interactions, or a combination thereof when the modified TCR is outside the tumor microenvironment.

3. The modified TCR of any one of claims 1-2, wherein P₃ is bound to T₃ at or near the antigen binding site when the modified TCR is outside the tumor microenvironment.

4. The modified TCR of any one of claims 1-3, wherein P₃ inhibits the binding of T₃ to the target antigen when the modified TCR is outside the tumor microenvironment, and P₃ does not inhibit the binding of T₃ to the target antigen when the modified TCR is inside the tumor microenvironment.

5. The modified TCR of any one of claims 1-4, wherein P₃ sterically blocks T₃ from binding to the target antigen when the modified TCR is outside the tumor microenvironment.

6. The modified TCR of any one of claims 1-5, wherein P₃ is removed from the antigen binding site, and the antigen binding site of T₃ is exposed when the modified TCR is inside the tumor microenvironment.

7. The modified TCR of any one of claims 1-6, wherein P₃ comprises less than 70% sequence homology to the target antigen.

8. The modified TCR of any one of claims 1-7, wherein P₃ comprises a peptide sequence of at least 10 amino acids in length.

9. The modified TCR of any one of claims 1-8, wherein P₃ comprises a peptide sequence of at least 10 amino acids in length and no more than 20 amino acids in length.

10. The modified TCR of any one of claims 1-9, wherein P₃ comprises a peptide sequence of at least 16 amino acids in length.

11. The modified TCR of any one of claims 1-10, wherein P₃ comprises at least two cysteine amino acid residues.

12. The modified TCR of any one of claims 1-10, wherein P₃ comprises an amino acid sequence according to SEQ ID NO: 59 (YDXXF), wherein X is any amino acid.

13. The modified TCR of any one of claims 1-10, wherein P₃ comprises an amino acid sequence according to SEQ ID NO: 59 (YDXXF), wherein X is any amino acid except for cysteine.

14. The modified TCR of any one of claims 1-12, wherein P₃ comprises an amino acid sequence according to SEQ ID NO: 60 (DVYDEAF).

15. The modified TCR of any one of claims 1-12, wherein P₃ comprises an amino sequence according to SEQ ID NO: 61 (GGVSCKDVYDEAFCWT).

16. The modified TCR of any one of claims 1-15, wherein P₃ comprises a cyclic peptide or a linear peptide.

17. The modified TCR of any one of claims 1-16, wherein P₃ comprises a cyclic peptide.

18. The modified TCR of any one of claims 1-16, wherein P₃ comprises a linear peptide.

19. The modified TCR of any one of claims 1-18, wherein L₃ is a peptide sequence having at least 5 to no more than 50 amino acids.

20. The modified TCR of any one of claims 1-19, wherein L₃ is a peptide sequence having at least 10 to no more than 30 amino acids.

21. The modified TCR of any one of claims 1-20, wherein L₃ is a peptide sequence having at least 10 amino acids.

22. The modified TCR of any one of claims 1-21, wherein L₃ is a peptide sequence having at least 18 amino acids.

23. The modified TCR of any one of claims 1-22, wherein L₃ is a peptide sequence having at least 26 amino acids.

24. The modified TCR of any one of claims 1-23, wherein L₃ has a formula comprising (G₂S)ₙ, wherein n is an integer from 1 to 3 (SEQ ID NO: 64).

25. The modified TCR of any one of claims 1-24, wherein L₃ is a substrate for a tumor specific protease.

26. The modified TCR of claim 25, wherein the tumor specific protease is selected from the group consisting of metalloprotease, serine protease, cysteine protease, threonine protease, and aspartic protease.

27. The modified TCR of any one of claims 1-26, wherein L₃ comprises a urokinase cleavable amino acid sequence, a MT-SP1 cleavable amino acid sequence, or a KLK5 cleavable amino acid sequence.

28. The modified TCR of any one of claims 1-27, wherein L₃ comprises an amino acid sequence according to SEQ ID NO: 62 (GGGGSLSGRSDNHGSSGT).

29. The modified TCR of any one of claims 1-27, wherein L₃ comprises an amino acid sequence according to SEQ ID NO: 63 (GGGGSSGGSGGSGLSGRSDNHGSSGT).

30. The modified TCR of any one of claims 1-29, wherein T₃ comprises a MAGE-A3 domain.

31. The modified TCR of any one of claims 1-30, wherein T₃ comprises a MAGE-A3 alpha domain.

32. The modified TCR of any one of claims 1-30, wherein T₃ comprises a MAGE-A3 beta domain.

33. The modified TCR of any one of claims 1-30, wherein T₃ comprises an amino acid sequence according to SEQ ID NO: 46.

34. The modified TCR of any one of claims 1-30, wherein T₃ comprises an amino acid sequence according to SEQ ID NO: 47.

35. The modified TCR of any one of claims 1-30, wherein T₃ comprises an amino acid sequence according to SEQ ID NO: 54.

36. The modified TCR of any one of claims 1-30, wherein T₃ comprises an amino acid sequence according to SEQ ID NO: 55.

37. The modified TCR of any one of claims 1-36, wherein T₃ comprises the TCR alpha extracellular domain, or fragment thereof, and the modified TCR further comprises a second polypeptide comprising a TCR beta extracellular domain, or a fragment thereof wherein the TCR beta extracellular domain or fragment thereof contains an antigen binding site.

38. The modified TCR of any one of claims 1-36, wherein T₃ comprises the TCR beta extracellular domain, or fragment thereof, and the modified TCR further comprises a second polypeptide comprising a TCR alpha extracellular domain, or a fragment thereof wherein the TCR alpha extracellular domain or fragment thereof contains an antigen binding site.

39. The modified TCR of any one of claims 1-38, wherein the TCR alpha extracellular domain, or fragment thereof, comprises three hypervariable complementarity determining regions (CDRs).

40. The modified TCR of claim 39, wherein at least one CDR comprises a mutation to increase binding affinity or binding specificity to the target antigen or to increase binding affinity and binding specificity to the target antigen.

41. The modified TCR of any one of claims 1-40, wherein the TCR beta extracellular domain, or fragment thereof, comprises three hypervariable complementarity determining regions (CDRs).

42. The modified TCR of claim 41, wherein at least one CDR comprises a mutation to increase binding affinity or binding specificity to the target antigen or to increase binding affinity and binding specificity to the target antigen.

43. The modified TCR of any one of claims 37-42, wherein the TCR alpha extracellular domain, or fragment thereof, and the TCR beta extracellular domain, or fragment thereof, are connected by a disulfide bond.

44. The modified TCR of any one of claims 37-43, wherein the TCR alpha extracellular domain, or fragment thereof, comprises an alpha chain TRAC constant domain sequence and the TCR beta extracellular domain, or fragment thereof, comprises a beta chain TRBC1 or TRBC2 constant domain sequence.

45. The modified TCR of claim 44, wherein Cys4 of the alpha chain TRAC constant domain sequence is modified by truncation or substitution and Cys2 of exon 2 of the beta chain TRBC1 or TRBC2 constant domain sequence is modified by truncation or substitution, thereby deleting a native disulfide bond.

46. The modified TCR of claim 44 or 45, wherein Thr48 of the alpha chain TRAC constant domain sequence is mutated to Cys and Ser57 of the beta chain TRBC1 or TRBC2 constant domain sequence is mutated to Cys.

47. The modified TCR of any one of claims 1-46, wherein the modified TCR comprises a modified amino acid, a non-natural amino acid, a modified non-natural amino acid, or a combination thereof.

48. The modified TCR of claim 47, wherein the modified amino acid or modified non-natural amino acid comprises a post-translational modification.

49. The modified TCR of any one of claims 1-48, wherein the target antigen is MAGE-A3 or titin.

50. The modified TCR of any one of claims 1-49, wherein the polypeptide of formula III binds to a target cell when L₃ is cleaved by the tumor specific protease.

51. The modified TCR of any one of claims 1-50, wherein P₃ inhibits binding of the modified TCR to the target cell when outside the tumor microenvironment.

52. The modified TCR of any one of claims 1-51, wherein the modified TCR has an increased binding affinity for its pMHC as compared to the binding affinity for the pMHC of an unmodified form of the TCR that does not have P₃ or L₃.

53. The modified TCR of any one of claims 1-52, wherein the modified TCR has an increased binding affinity for its pMHC that is at least 10× higher than the binding affinity for the pMHC of an unmodified form of the TCR that does not have P₃ or L₃.

54. The modified TCR of any one of claims 1-53, wherein the modified TCR has an increased binding affinity for its pMHC that is at least 100× higher than the binding affinity for the pMHC of an unmodified form of the TCR that does not have P₃ or L₃.

55. The modified TCR of any one of claims 1-51, wherein the modified TCR has an increased binding affinity for its pMHC as compared to the binding affinity for the pMHC of the modified TCR in which L₃ has been cleaved by the tumor specific protease.

56. The modified TCR of any one of claims 1-55, wherein the modified TCR has an increased binding affinity for its pMHC that is at least 10× higher than the binding affinity for the pMHC of the modified TCR in which L₃ has been cleaved by the tumor specific protease.

57. The modified TCR of any one of claims 1-56, wherein the modified TCR has an increased binding affinity for its pMHC that is at least 100× higher than the binding affinity for the pMHC of the modified TCR in which L₃ has been cleaved by the tumor specific protease.

58. A pharmaceutical composition, comprising:
(a) the modified TCR according to claims 1-57; and
(b) a pharmaceutically acceptable excipient.

59. An isolated recombinant nucleic acid molecule encoding a polypeptide comprising a formula III:
T₃-L₃-P₃ (formula III)
wherein:
T₃ comprises either a TCR alpha extracellular domain, or fragment thereof, or a TCR beta extracellular domain, or fragment thereof, wherein T₃ binds to a target antigen and the TCR alpha extracellular domain or fragment thereof and the TCR beta extracellular domain, or fragment thereof contain an antigen binding site,
P₃ is a peptide that reduces binding of T₃ to the target antigen when the modified TCR is outside of a tumor microenvironment and that does not reduce binding of T₃ to the target antigen when the modified TCR is inside the tumor microenvironment, and
L₃ is a linking moiety that connects T₃ to P₃ and L₃ is bound to T₃ at the N-terminus of T₃,
wherein the modified TCR is a soluble TCR and is a functional TCR when inside the tumor microenvironment and is a nonfunctional TCR when outside the tumor microenvironment and P₃ or L₃ is a substrate for a tumor specific protease.
